(19) United States
(12) Patent Application Publication
(10) Pub. No.: US 2021/0340629 A1
(43) Pub. Date: Nov. 4, 2021

(54) BIOMARKERS FOR CANCER THERAPY

(71) Applicant: The Council of the Queensland Institute of Medical Research, Herston (AU)

(72) Inventors: Jason Lee, Herston (AU); Fares Al-Ejeh, Herston (AU)

(73) Assignee: The Council of the Queensland Institute of Medical Research, Herston (AU)

(21) Appl. No.: 17/274,126

(22) PCT Filed: Sep. 6, 2019
(86) PCT No.: PCT/AU2019/050953

§ 371 Date: Mar. 5, 2021

(30) Foreign Application Priority Data
Sep. 6, 2018 (AU) 2018903318

(51) Int. Cl. C12Q 1/6886; G01N 33/574

(52) U.S. Cl. CPC C12Q 1/6886; G01N 33/5743; G01N 2800/52; C12Q 2600/158; C12Q 2600/106

(57) ABSTRACT

This invention relates generally to biomarkers that are useful for determining whether a subject with cancer is likely to respond to cancer therapy. The invention therefore relates to methods, kits and compositions for determining whether a subject is likely to respond to cancer therapy, and to methods of treatment based on a determination that a subject with cancer is likely to respond to cancer therapy. The invention also relates to methods for sensitizing a subject with cancer to cancer therapy.

### FIELD

This invention relates generally to biomarkers that are useful for determining whether a subject with cancer is likely to respond to cancer therapy. The invention therefore relates to methods, kits and compositions for determining whether a subject is likely to respond to cancer therapy, and to methods of treatment based on a determination that a subject with cancer is likely to respond to cancer therapy. The invention also relates to methods for sensitizing a subject with cancer to cancer therapy.

### RELATED APPLICATIONS

This application claims priority to Australian Provisional Application No. 2018903318 entitled “Biomarkers for cancer therapy” filed 6 Sep. 2018, the contents of which are incorporated herein by reference in their entirety.

### BACKGROUND

Cancer therapies have evolved significantly over time, from generalized radiotherapy and chemotherapy to the more recent targeted therapies and immunotherapies. Of these newer cancer therapies, one of the most exciting and promising classes has been the group of molecules known as immune checkpoint inhibitors. Immune checkpoint inhibitors, which to date have been antibodies, block specific interactions between immune checkpoint molecules, leading to a reversal of the downregulation of the immune system that these interactions normally have in the tumor environment. Immune checkpoint inhibitors that block the interaction between cytotoxic T-lymphocyte associated protein 4 (CTLA-4) on cytotoxic T cells and cluster differential 80 (CD80)/cluster differential 86 (CD86) on antigen presenting cells (APC), or the interaction between programmed cell death-1 (PD-1) on cytotoxic T cells and programmed death ligand-1 (PD-L1) on APC, reactivate the anti-tumor immune response, resulting in improved survival outcomes for patients with various types of cancers. However, while immune checkpoint inhibitors have shown remarkable efficacy in some patients, there is a significant proportion of patients who do not respond to these costly treatments. There remains a need, therefore, for improved means for determining which patients are likely to respond to cancer therapy with immune checkpoint inhibitors, so to optimize treatment for those patients and to reduce exposure to unnecessary therapy, which can have harmful side-effects, in other patients. There also remains a need for methods for sensitizing subjects to these therapies, so as to improve the degree to which a subject responds to the therapy, and/or to increase the number of subjects responding to the therapy.

### SUMMARY

The present invention is predicated in part on the determination that expression products of the MAP1LC3B gene are reliable indicators of response to cancer therapy, and in particular therapy with an immune checkpoint inhibitor. Therefore, the present inventors determined that MAP1LC3B is a reliable biomarker of an increased or decreased likelihood that a subject with cancer will respond to therapy with an immune checkpoint inhibitor. The present inventors have also determined that expression products of EHMT2 are also indicative of response to therapy, and thus can be included in the diagnostic and prognostic assays taught herein. Based on these determinations, it is proposed that the concentration, level or abundance of expression products of MAP1LC3B, optionally in combination with the concentration, level or abundance of expression products of EHMT2, is indicative of the ability of a subject with cancer to respond to cancer therapy, and has utility for determining whether a subject is likely to respond to cancer therapy, and in particular therapy with an immune checkpoint inhibitor. MAP1LC3B, optionally in combination with EHMT2, therefore has utility as a biomarker for stratifying or classifying subjects as those who are likely to be responders (i.e. likely to exhibit a positive response to the cancer therapy), and those who are likely to be non-responders (i.e. likely to have no response or a negative response to the cancer therapy). Moreover, as taught herein, those subjects who are classified as responders can be further classified as complete responders or partial responders. Based on the experimental findings provided herein, the present inventors also provide methods for sensitizing a subject with cancer to a cancer therapy, and in particular therapy with an immune checkpoint inhibitor, and methods for treating a subject with cancer.

Accordingly, in one aspect, the present invention provides a method for determining an indicator used in assessing a likelihood of a subject with cancer responding to cancer therapy, the method comprising, consisting or consisting essentially of: (1) determining a biomarker value for at least one cancer therapy biomarker in a sample from the subject, wherein the, or one of the, cancer therapy biomarker(s) is an expression product of MAP1LC3B; and (2) determining the indicator using the biomarker value(s), wherein the indicator is at least partially indicative of the likelihood of responsiveness to cancer therapy; wherein the cancer therapy comprises therapy with an immune checkpoint inhibitor.

In some embodiments, the expression product of MAP1LC3B is a polynucleotide and the biomarker value for MAP1LC3B is indicative of the abundance or concentration of the polynucleotide in the sample. In such embodiments, the polynucleotide expression product may comprise a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence set forth in SEQ ID NO:1, or a complement thereof. In other embodiments, the expression product of MAP1LC3B is a polypeptide and the biomarker value for MAP1LC3B is indicative of the abundance or concentration of the polypeptide in the sample. in such embodiments, the polypeptide expression product may comprise an amino acid sequence having at least 85%, 86%, 87%, 88%, 89%, 90%, 91%, 92%, 93%, 94%, 95%, 96%, 97%, 98% or 99% sequence identity with the sequence set forth in SEQ ID NO:2.

In some instances, the abundance or concentration of the polynucleotide or polypeptide expression product of MAP1LC3B is increased relative to the abundance or concentration that correlates with a negative response to cancer therapy, and the indicator is thereby determined to be at least partially indicative of a positive response (e.g. complete or partial response) to therapy; the abundance or concentration of the polynucleotide or polypeptide expression product of MAP1LC3B is about the same as the abundance or concentration that correlates with a positive response (e.g. complete or partial response) to cancer therapy, and the indicator is thereby determined to be at least partially indicative of a positive response (e.g. complete or partial response) to therapy; the abundance or concentration of the polynucleotide or polypeptide expression product of MAP1LC3B is about the same as the abundance or concentration that correlates with a negative response to cancer therapy, and the indicator is thereby determined to be at least partially indicative of a negative response to therapy; or the abundance or concentration of the polynucleotide or polypeptide expression product of MAP1LC3B is decreased relative the abundance or concentration that correlates with a positive response (e.g. complete or partial response) to cancer therapy, and the indicator is thereby determined to be at least partially indicative of a negative response to therapy.

In further embodiments of the methods of the present invention, one of the at least one cancer therapy biomarkers is an expression product of EHMT2. For example, the expression product of EHMT2 may be a polynucleotide and the biomarker value for EHMT2 is indicative of the abundance or concentration of the polynucleotide in the sample. In such examples, the polynucleotide expression product may comprise a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the sequence set forth in any one of SEQ ID NOs:3, 5, 7, 9 and 11, or a complement thereof. In other examples, the expression product of EHMT2 is a polypeptide and the biomarker value for EHMT2 is indicative of the abundance or concentration of the polypeptide in the sample. In such examples, the polypeptide expression product may comprise an amino acid sequence having at least 85%, 86%, 87%, 88%, 89%, 90%, 91%, 92%, 93%, 94%, 95%, 96%, 97%, 98% or 99% sequence identity with the sequence set forth in any one of SEQ ID NOs:4, 6, 8, 10 and 12. In a particular embodiment, the biomarker value for the expression product of EHMT2 is determined by measuring the abundance or concentration of an expression product of EHMT1.

In particular embodiments of these methods, the abundance or concentration of the polynucleotide or polypeptide expression product of EHMT2 is increased relative to the abundance or concentration that correlates with a positive response (e.g. complete or partial response) to cancer therapy, and the indicator is thereby determined to be at least partially indicative of a negative response to therapy; the abundance or concentration of the polynucleotide or polypeptide expression product of EHMT2 is about the same as the abundance or concentration that correlates with a negative response to cancer therapy, and the indicator is thereby determined to be at least partially indicative of a negative response to therapy; the abundance or concentration of the polynucleotide or polypeptide expression product of EHMT2 is decreased relative to the abundance or concentration that correlates with a negative response to cancer therapy, and the indicator is thereby determined to be at least partially indicative of a positive response (e.g. complete or partial response) to therapy; or the abundance or concentration of the polynucleotide or polypeptide expression product of EHMT2 is about the same as the abundance or concentration that correlates with positive response to cancer therapy, and the indicator is thereby determined to be at least partially indicative of positive response (e.g. complete or partial response) to therapy. In one specific example, the abundance or concentration of the polynucleotide or polypeptide expression product of MAP1LC3B is decreased relative to the abundance or concentration that correlates with positive response (e.g. complete or partial response) to cancer therapy; the abundance or concentration of the polynucleotide or polypeptide expression product of EHMT2 is increased relative the abundance or concentration that correlates with positive response to cancer therapy; and the indicator is thereby determined to be at least partially indicative of a negative response to therapy.

In particular examples of the methods of the invention, the indicator is derived from a ratio of the abundance or concentration of the polynucleotide or polypeptide expression product of MAP1LC3B to the abundance or concentration of the polynucleotide or polypeptide expression product of EHMT2. In some examples, the ratio is higher relative to a ratio that correlates with a negative response to therapy, and the indicator is thereby determined to be at least partially indicative of a positive response to therapy; the ratio is about the same as a ratio that correlates with a positive response to therapy, and the indicator is thereby determined to be at least partially indicative of a positive response to therapy; the ratio is lower relative to a ratio that correlates with a positive response to therapy, and the indicator is thereby determined to be at least partially indicative of a negative response to therapy; or the ratio is about the same as a ratio that correlates with a negative response to therapy, and the indicator is thereby determined to be at least partially indicative of a negative response to therapy.

In further examples, the positive response to therapy is a complete or partial response to therapy. For example, in some embodiments, the ratio is higher relative to a ratio that correlates with a partial response to therapy, and the indicator is thereby determined to be at least partially indicative of a complete response to cancer therapy; the ratio is about the same as a ratio that correlates with a complete response to therapy, and the indicator is thereby determined to be at least partially indicative of a complete response to cancer therapy; the ratio is lower relative to a ratio that correlates with a complete response to therapy, and the indicator is thereby determined to be at least partially indicative of a partial response to therapy; the ratio is about the same as a ratio that correlates with a partial response to therapy, and the indicator is thereby determined to be at least partially indicative of a partial response to therapy; the ratio is lower relative to a ratio that correlates with a partial response to therapy, and the indicator is thereby determined to be at least partially indicative of a negative response to therapy; or the ratio is about the same as a ratio that correlates with a negative response to therapy, and the indicator is thereby determined to be at least partially indicative of a negative response to therapy.

In a further embodiment, the at least one cancer therapy biomarker comprises LDH, BRAF and NRAS. The biomarker value for serum LDH may be determined by measuring the abundance or concentration of serum LDH. The biomarker value for BRAF and NRAS is a BRAF/NRAS mutation status, wherein the BRAF/NRAS mutation status is determined by detecting the presence or absence of mutations in BRAF and NRAS, whereby a detection of one or more mutations in BRAF and NRAS is a positive BRAF/NRAS mutation status, and the detection of no mutations in BRAF and NRAS is a positive BRAF/NRAS mutation status.

In one example, the abundance or concentration of the polynucleotide or polypeptide expression product of MAP1LC3B is increased relative to a reference level; the abundance or concentration of serum LDH is the same as that of a healthy subject; the BRAF/NRAS mutation status is negative or positive; and the indicator is thereby determined to be at least partially indicative of a complete response to therapy; or the abundance or concentration of the polynucleotide or polypeptide expression product of MAP1LC3B is increased relative to a reference level; the abundance or concentration of serum LDH is increased relative to that of a healthy subject; the BRAF/NRAS mutation status is negative; and the indicator is thereby determined to be at least partially indicative of a complete response to therapy.

In another example, the abundance or concentration of the polynucleotide or polypeptide expression product of MAP1LC3B is decreased relative to a reference level; the abundance or concentration of serum LDH correlates with that of a healthy subject; the BRAF/NRAS mutation status is negative; and the indicator is thereby determined to be at least partially indicative of a partial response to therapy.

In a further example, the abundance or concentration of the polynucleotide or polypeptide expression product of MAP1LC3B is increased relative to a reference level; the abundance or concentration of serum LDH is increased relative to that of a healthy subject; the BRAF/NRAS mutation status is positive; and the indicator is thereby determined to be at least partially indicative of a negative response to therapy; the abundance or concentration of the polynucleotide or polypeptide expression product of MAP1LC3B is decreased relative to a reference level; the abundance or concentration of serum LDH is increased relative to that of a healthy subject; the BRAF/NRAS mutation status is negative or positive; and the indicator is thereby determined to be at least partially indicative of a negative response to therapy; or the abundance or concentration of the polynucleotide or polypeptide expression product of MAP1LC3B is decreased relative to a reference level; the abundance or concentration of serum LDH correlates with that of a healthy subject; the BRAF/NRAS mutation status is positive; and the indicator is thereby determined to be at least partially indicative of a negative response to therapy.

In the methods of the present invention, the biomarker value(s) may be measured using nucleic acid amplification techniques, sequencing platforms, array and hybridization platforms, microscopy, flow cytometry, immunoassays, mass spectrometry, or a combination thereof. In one example, the biomarker value(s) is(are) measured using quantitative RT-PCR. Moreover, the sample may comprise cancer or tumor cells.

A further aspect of the invention provides a method for treating cancer in a subject, the method comprising, consisting or consisting essentially of: performing the method described above and herein for determining an indicator used in assessing a likelihood of a subject with cancer responding to cancer therapy; and exposing the subject to a cancer therapy on the basis that the indicator is at least partially indicative of a positive response to cancer therapy; wherein the cancer therapy comprises therapy with an immune checkpoint inhibitor.

Also provided is a method for sensitizing a subject with cancer to cancer therapy, the method comprising, consisting or consisting essentially of performing the method described above and herein for determining an indicator used in assessing a likelihood of a subject with cancer responding to cancer therapy; and administering an EHMT2 or EHMT1 inhibitor to the subject on the basis that the indicator is at least partially indicative of a negative response to cancer therapy; wherein the cancer therapy comprises therapy with an immune checkpoint inhibitor. The EHMT2 inhibitor may be selected from among a small molecule (e.g. A-366, BIX01294, BRD4770, CM-272, E72, UNCO224, UNC0321, UNC0638, UNC0642, UNC0646, and verticillin A), an antibody or antigen-binding fragment thereof specific, an aptamer, and a nucleic acid molecule. In some embodiments, the method further comprises exposing the subject to the cancer therapy.

In some embodiments of the methods of the present invention, the immune checkpoint inhibitor is selected from among a CTLA-4 inhibitor (e.g. ipilimumab or tremelimumab), PD-1 inhibitor (e.g. pembrolizumab, pidilizumab, nivolumab, REGN2810, CT-001, AMP-224, BMS-936558, MK-3475, MEDI0680 and PDR001) and PD-L1 inhibitor (e.g. atezolizumab, durvalumab, avelumab, BMS-936559 and MEDI4736) or a combination thereof.

In particular examples of the methods for treating cancer or the methods for sensitizing a subject with cancer to cancer therapy, the cancer therapy comprises a further chemotherapeutic agent and/or radiotherapy.

The methods of the present invention are be performed with a subject with cancer. In particular embodiments, the cancer is a solid tumor. In other embodiments, the cancer is a blood tumor.

Also provided is a composition for determining an indicator used in assessing a likelihood of a subject with cancer responding to cancer therapy, the composition or solid support comprising, consisting, or consisting essentially of a MAP1LC3B transcript or cDNA thereof and at least one oligonucleotide primer or probe that hybridizes to the MAP1LC3B transcript or cDNA thereof, and an EHMT2 or EHMT1 transcript or cDNA thereof and at least one oligonucleotide primer or probe that hybridizes to the EHMT2 or EHMT1 transcript or cDNA thereof, wherein the cancer therapy comprises therapy with an immune checkpoint inhibitor. The cDNA may correspond to mRNA derived from a cell or cell population (e.g. is a tumor cell or tumor cell population).

In a further aspect, provided is a solid support for determining an indicator used in assessing a likelihood of a subject with cancer responding to cancer therapy, the solid support comprising, consisting, or consisting essentially of at least one first oligonucleotide primer or probe immobilized to the solid support, wherein the at least one first oligonucleotide primer or probe hybridizes to a MAP1LC3B transcript or cDNA; and at least one second oligonucleotide primer or probe immobilized to the solid support, wherein the at least one second oligonucleotide primer or probe hybridizes to a EHMT2 t or EHMT1 transcript or cDNA thereof, wherein the cancer therapy comprises therapy with an immune checkpoint inhibitor. In one embodiment, the support further comprises a MAP1LC3B transcript or cDNA thereof hybridized to the at least one first oligonucleotide primer or probe; and an EHMT2 or EHMT1 transcript or cDNA thereof hybridized to the at least one second oligonucleotide primer or probe. The cDNA may correspond to mRNA derived from a cell or cell population (e.g. is a tumor cell or tumor cell population).

In another aspect of the invention, provided is a composition for determining an indicator used in assessing a likelihood of a subject with cancer responding to cancer therapy, the composition comprising, consisting, or consisting essentially of tumor cells, a detection agent that binds to the polypeptide expression product of MAP1LC3B, and a detection agent that binds to the polypeptide expression product of EHMT2 or EHMT1, wherein the cancer therapy comprises therapy with an immune checkpoint inhibitor. In some examples, the detection agents are antibodies or antigen-binding fragments thereof.

Also provided is a kit for determining an indicator used in assessing a likelihood of a subject with cancer responding to cancer therapy, the kit comprising, consisting, or consisting essentially of (a) at least one reagent that allows quantification of a polynucleotide or polypeptide expression product of MAP1LC3B in a biological sample; and optionally (b) instructions for using the at least one reagent; wherein the cancer therapy comprises therapy with an immune checkpoint inhibitor. In one embodiment, the kit further comprises at least one reagent that allows quantification of a polynucleotide or polypeptide expression product of EHMT2 or EHMT1 in the biological sample.

### BRIEF DESCRIPTION OF THE DRAWINGS

FIG. 1 shows an image of immunoblotting analysis of EHMT2 (G9a) from eight tested melanoma cell lines as well as the normal melanocytes (Norm). Tubulin was used as a loading control.

FIG. 2 is a graphical representation of cell viability of melanoma cell lines treated with either vehicle (DMSO) or 5 μM of UNC0642 for 48 hours. Cell viability was measured by MTT assay against normal melanocytes.

FIG. 3 represents the results of a study of four melanoma cell lines (D05 (BRAF mutant), C006 (NRAS mutant), C008 (NF1 mutant) and C092 (Triple wild type)) treated with either vehicle (DMSO) or 5 μM of UNC0642 for 48 hours. (A) Graphical representation of relative proliferation of cells as assessed using IncuCyte ZOOM time-lapse imaging analysis. (B) Image of immunoblotting analysis of H3K9me2 from D05 and C008, with total H3 used as loading control.

FIG. 4 represents the results of a study assessing cellular proliferation and viability of the D05 cell line following knock down of G9a using shG9a or a non-silencing control (shNS). (A) Cellular proliferation as analyzed by IncuCyte ZOOM imaging. (B) Cell viability as assessed by MTT assay. Data is presented as the mean±SEM, significant comparisons were determined by unpaired t-test and indicated as follows: *P<0.05, **P<0.01, (all experiments were performed twice with between 3 and 6 replicates per experiment).

FIG. 5 shows an image of immunoblotting analysis of LC3B I/II and ATG5 from various melanoma cell lines as well as normal melanocytes. Tubulin levels were used as a loading control.

FIG. 6 shows an image of Immunoblotting analysis of LC3B I/II from melanoma cell lines treated with either vehicle (−) or 5 μM of UNC0642. Tubulin was used as a loading control.

FIG. 7 shows an image of immunoblotting analysis of G9a and LC3B I/II from D05 and C092 melanoma cell lines expressing shNS (−) and shG9a (+). Tubulin levels were used as a loading control.

FIG. 8 is a graphical representation of expression levels of MAP1LC3B in four melanoma cell lines treated with UNC0642 for 24 hours, as assessed by quantitative RT-PCR. The results are expressed as fold change compared to vehicle control (DMSO).

FIG. 9 represents the results of a chromatin immunoprecipitation analysis of H3K9me2, Pol II on the MAP1LC3B promoter (left panel) or 5 kb upstream of the MAP1LC3B promoter (right panel) in D05 cells treated with UNC0642 (5 μM, 24 h), *P<0.05, **P<0.01, n=3.

FIG. 10 represents the results of a study assessing the effect of a G9a inhibitor on tumors in mice. Groups of SCID mice (n=6-9) were subcutaneously injected with D20 melanoma cells (2×10⁵) on day 0. Tumor-bearing mice were treated with vehicle (DMSO) or 5 mg/kg UNC0642 intraperitoneally every two days. (A) Tumor growth was measured using a digital caliper, and tumor volumes are represented as mean±SEM. Statistical differences in tumor volumes were determined by unpaired t-test (*P<0.05). (B) Tumor weight at end-point represented as mean±SEM. (C) MAP1LC3B gene expression from tumors extracted from mice treated with either vehicle or UNC0642 determined using qRT-PCR. Data is shown as the mean±SEM. Statistical differences were determined by unpaired t-test, **P<0.01. (D) Immunohistochemical analysis of MAP1LC3B protein expression in D20 xenografts excised from mice. Stained tumor sections were subdivided into 5 non-overlapping regions of the stained tumor sections were analyzed for their number of positive stained pixels and quantified per unit area (μm²) using the Aperio ImageScope software. Results are summarized as bar graphs and are shown as the mean±SEM. Statistical differences were determined by unpaired t-test, *P<0.01, n=5.

FIG. 11 represents the results of an analysis of EHMT2 and MAP1LC3B expression in the TCGA melanoma RNA-seq dataset. (A) Co-expression of EHMT2 (G9a) and MAP1LC3B mRNA (z-scores) in the TCGA melanoma patient cohort (n=473). Pearson correlation coefficient (Pearson r) and P value (two-tailed) were generated by GraphPad Prism. Patients were grouped according to their EHMT2 and MAP1LC3B mRNA expression; EHMT2 high/MAP1LC3B low (EHMT2^{hi}/MAP1LC3B^{lo}), EHMT2 high/MAP1LC3B high (EHMT2^{hi}/MAP1LC3B^{hi}), EHMT2^{lo}/MAP1LC3B^{hi} and EHMT2^{lo}/MAP1LC3B^{lo}. (B) Overall survival of melanoma patients with four different expression patterns of EHMT2 and MAP1LC3B. Log-rank P values and the number of patients in each group are reported. (C) Relapse-free survival of melanoma patients with four different expression patterns of EHMT2 and MAP1LC3B. Log-rank P values and the number of patients in each group are reported. (D) Comparison of BRAF, NRAS, NF1 and Triple wt mutation status across the four EHMT2/MAP1LC3B expression groups. Chi square test was used (GraphPad Prism).

FIG. 12 shows G9a and/or LC3B expression in melanoma patients. (A) Diagram of pre-treatment tumor biopsy sample collection from metastatic melanoma patients for MAP1LC3B transcript analysis. (B) Survival curve for metastatic melanoma patients using tumor MAP1LC3B transcript levels. Survival of two patient groups, MAP1LC3B high (grey) and MAP1LC3B low (black) are shown. (C) Graph representing the relative gene expression of EHMT2 and MAP1LC3B from the two patient groups (pre-treatment). (D) Diagram of pre-treatment tumor biopsy samples from metastatic melanoma patients for TMA processing and IHC analysis. (E) Graphical representation of the number and mean intensity of the LC3B staining in the TMA sections by Responder and Non-responder. P<0.05.

FIG. 13 shows Receiver Operator Characteristic (ROC) curves of LC3B as a predictor of outcome in melanoma patients treated with immunotherapy. ROC curves were constructed using MedCalc® (version 12.7) for all the endpoints available in our cohort including survival (dead vs. alive), response (initial SD/PD vs. CR/PR), progression (de novo or acquired PD vs. rest), and acquired resistance (acquired PD vs. rest). The endpoint analysis is displayed for (A), the percentage of LC3-positive cells (% LC3B+ cells) or (B), absolute LC3B staining intensity (LC3B expression).

FIG. 14 shows patients were classified based on the (A) percentage of LC3B-positive cells (% LC3B⁺ cells) or (B) absolute LC3B staining intensity (LC3B expression). The classification was based on the cut-offs from ROC curves to define low (black) or high (grey) groups. KM plots are shown for overall survival, response (CR or PR), Progression (de novo or post-response), and for acquired resistance (PD after initial response). Hazard ratios and P-values from log-rank (Mantel-Cox) test using GraphPad Prism are shown for each plot. Total number of patients=40; 16 patients had % LC3B+ cells>18.5% and 15 patients had LC3B staining intensity>753.31 (absolute units).

FIG. 15 shows G9a and LC3B expression in CTCs from melanoma patients. (A) Diagram of post-treatment liquid biopsy sample collection from metastatic melanoma patients for CTC G9a and LC3B protein analysis. (B) Graphical representation of the ratio of total fluorescence intensity of LC3B to G9a in CTCs. ****P<0.0001.

FIG. 16 shows a model for G9a inhibitor-mediated re-expression of MAP1LC3B for melanoma cell death induction and utility of G9a and MAP1LC3B as patient selection and immune checkpoint inhibitor (ICI) therapy response markers. (A) G9a inhibitor induces MAP1LC3B expression by reducing histone H3K9 methylation, thereby modulates autophagy and leads to better response to ICI therapy. (B) G9a and MAP1LC3B levels can stratify melanoma patients into distinct prognostic groups. Melanoma patients with low G9a and high LC3B should response to standard ICI therapy, however, patients with high G9a and low LC3B, who would inherently be resistant to standard immune checkpoint inhibitor therapy, can be treated with G9a inhibitor and the resultant decrease in G9a activity and increase in MAP1LC3B expression would sensitise these patients to standard immune checkpoint inhibitor therapy.

FIG. 17 shows outcomes of melanoma patients of different classifications. (A) Outcomes of patients classified by percentage of LC3B-positive cells and BRAF/NRAS mutation status. (B) Outcomes of patients classified by percentage of LC3B-positive cells and LDH status. (C) Classification of patients into Groups 1-3. (D) Outcomes of patients classified into Groups 1-3.

FIG. 18 represents an analysis of immune effector cells from AT3 tumors that were exposed to vehicle or UNC0642 at 25 mg/kg every 2 days for 14 days. Tumor infiltrating lymphocytes (TILs) and NK cells were then analyzed by FACs. (A) Frequency (%) of CD4⁺ T cells. (B) Frequency (%) of CD8⁺PD-1⁺ T cells, and CD8⁺PDL-1⁺ T cells. (C) Frequency (%) of CD4⁺CD39⁺ T cells, and CD8⁺CD39⁺ T cells. (D) Frequency (%) of CD4⁺CD73⁺ T cells, and CD8⁺CD73⁺ T cells. (E) Number of NK cells per mg tissue. *P<0.05, **P<0.01, ***P<0.001, ****P<0.0001.

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein the terms “abundance,” “level” and “amount” are used interchangeably herein to refer to a quantitative amount (e.g., weight or moles), a semi-quantitative amount, a relative amount (e.g., weight % or mole % within class), a concentration, and the like. Thus, these terms encompass absolute or relative amounts or concentrations of cancer therapy biomarkers in a sample.

As used herein, “and/or” refers to and encompasses any and all possible combinations of one or more of the associated listed items, as well as the lack of combinations when interpreted in the alternative (or).

The term “antibody” and its grammatical equivalents refer to a protein which is capable of specifically binding to a target antigen and includes any substance, or group of substances, which has a specific binding affinity for an antigen, suitably to the exclusion of other substances. This term encompasses an immunoglobulin molecule capable of specifically binding to a target antigen by virtue of an antigen binding site contained within at least one variable region. This term includes four chain antibodies (e.g., two light chains and two heavy chains), recombinant or modified antibodies (e.g., chimeric antibodies, humanized antibodies, primatized antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)). An antibody generally comprises constant domains, which can be arranged into a constant region or constant fragment or fragment crystallizable (Fc). In specific embodiments, the antibodies comprise a four-chain structure as their basic unit. Full-length antibodies comprise two heavy chains (≈50-70 kDa) covalently linked and two light chains (≈23 kDa each). A light chain generally comprises a variable region and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ≈110 amino acids in length) and one or more constant domains at the C-terminus. The constant domain of the light chain (C_{L} which is ≈110 amino acids in length) is aligned with and disulfide bonded to the first constant domain of the heavy chain (C_{H} which is ≈330-440 amino acids in length). The light chain variable region is aligned with the variable region of the heavy chain. The antibody heavy chain can comprise two or more additional CH domains (such as, C_{H2}, C_{H3} and the like) and can comprise a hinge region can be identified between the C_{H1} and Cm constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (suitably human) antibody. The term “antibody” encompasses not only intact polyclonal or monoclonal antibodies, but also variants, fusion proteins comprising an antibody portion with an antigen binding site, humanized antibodies, human antibodies, chimeric antibodies, primatized antibodies, de-immunized antibodies or veneered antibodies. Also within the scope of the term “antibody” are antigen binding fragments that retain specific binding affinity for an antigen, suitably to the exclusion of other substances. This term includes a Fab fragment, a Fab′ fragment, a F(ab′) fragment, a single chain antibody (SCA or SCAB) amongst others. An

“Fab fragment” consists of a monovalent antigen-binding fragment of an antibody molecule, and can be produced by digestion of a whole antibody molecule with the enzyme papain, to yield a fragment consisting of an intact light chain and a portion of a heavy chain. An “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An “F(ab′)2 fragment” of an antibody consists of a dimer of two Fab′ fragments held together by two disulfide bonds, and is obtained by treating a whole antibody molecule with the enzyme pepsin, without subsequent reduction. A (Fab′)₂ fragment. An “Fv fragment” is a genetically engineered fragment containing the variable region of a light chain and the variable region of a heavy chain expressed as two chains. A “single chain antibody” (SCA) is a genetically engineered single chain molecule containing the variable region of a light chain and the variable region of a heavy chain, linked by a suitable, flexible polypeptide linker.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “biomarker” broadly refers to any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular antibody can be indicative of the presence of a particular antigen (e.g., protein) in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional. In specific embodiments, the “biomarkers” include “cancer therapy biomarkers”, which are described in more detail below.

The term “biomarker value” refers to a value measured or derived for at least one corresponding biomarker of a subject and which is typically at least partially indicative of an abundance or concentration of a biomarker in a sample taken from the subject. Thus, the biomarker values could be measured biomarker values, which are values of biomarkers measured for the subject, or alternatively could be derived biomarker values, which are values that have been derived from one or more measured biomarker values, for example by applying a function to the one or more measured biomarker values. Biomarker values can be of any appropriate form depending on the manner in which the values are determined. For example, the biomarker values could be determined using high-throughput technologies such as sequencing platforms, array and hybridization platforms, mass spectrometry, immunoassays, immunofluorescence, flow cytometry, or any combination of such technologies. In one preferred example, the biomarker values relate to a level of abundance or activity of an expression product or other measurable molecule, quantified using a technique such as quantitative RT-PCR, sequencing, or the like. In this case, the biomarker values can be in the form of amplification amounts, or cycle times, which are a logarithmic representation of the concentration of the biomarker within a sample, as will be appreciated by persons skilled in the art and as will be described in more detail below. In other preferred examples, the biomarker values are quantified using immunofluorescence of cells containing the expression product.

The term “biomarker profile” refers to one or a plurality of one or more types of biomarkers (e.g., an mRNA molecule, a cDNA molecule and/or a protein, etc.), or an indication thereof, together with a feature, such as a measurable aspect (e.g., biomarker value) of the biomarker(s). A biomarker profile may comprise a single biomarker whose level, abundance or amount correlates with a condition or clinical state (e.g., responsiveness or non-responsiveness to cancer therapy). Alternatively, a biomarker profile may comprise at least two such biomarkers or indications thereof, where the biomarkers can be in the same or different classes, such as, for example, a nucleic acid and a polypeptide. Thus, a biomarker profile may comprise at least 2, 3, 4, 5, 6, 7, 8, 9, 10, 15, 20, 25, 30, 35, 40, 45, 50, 55, 60, 65, 70, 75, 80, 85, 90, 95, or 100 or more biomarkers or indications thereof. A biomarker profile can further comprise one or more controls or internal standards. In certain embodiments, the biomarker profile comprises at least one biomarker, or indication thereof, that serves as an internal standard. In other embodiments, a biomarker profile comprises an indication of one or more types of biomarkers. The term “indication” as used herein in this context merely refers to a situation where the biomarker profile contains symbols, data, abbreviations or other similar indicia for a biomarker, rather than the biomarker molecular entity itself. The term “biomarker profile” is also used herein to refer to a biomarker value or combination of at least two biomarker values, wherein individual biomarker values correspond to values of biomarkers that can be measured or derived from one or more subjects, which combination is characteristic of a condition or clinical state or a prognosis for a condition or clinical state (e.g., responsiveness or non-responsiveness to cancer therapy). The term “profile biomarkers” is used to refer to a subset of the biomarkers that have been identified for use in a biomarker profile that can be used in performing a clinical assessment, such as to rule in or rule out a specific conditions or clinical states. The number of profile biomarkers will vary, but is typically of the order of 10 or less. In one example, a biomarker profile includes a profile of biomarkers selected from an expression product of MAP1LC3B, an expression product of EHMT2, serum LDH, and BRAF/NRAS mutation status. Exemplary profiles therefore include a profile of an expression product of MAP1LC3B and an expression product of EHMT2, and a profile of expression product of MAP1LC3B, serum LDH, and BRAF/NRAS mutation status.

The term “BRAF/NRAS mutation status” refers to the status of a subject with respect to whether or not they have one or more mutations in the BRAF and NRAS genes, such as one or more mutations that affect the activity of the products of the genes (e.g. reduce or increase the activity of either one of the products of the genes by at least or about 20%, 30%, 40%, 50%, 60%, 70%, 80%, 90% or more) and are associated with cancer, e.g. melanoma. A negative BRAF/NRAS mutation status means that there are no mutations in either the BRAF or NRAS genes. Conversely, a positive BRAF/NRAS mutation status means that there is at least one mutation in the BRAF and/or NRAS gene. Exemplary mutations that affect the activity of the products of the genes include and have been associated with cancer include, but are not limited to, G7S, H57Y, F294L, S365L, G464E, S465Y, G466E, G469E, L496V, A497V, N581S, N581Y, L584S, D594G, L597S, A598A, V600E, V600K, V600R, K601E, V624F and T740A in BRAF, and G12S, G12D, G12A, G13R, G13V, A59D, Q61K, Q61R, Q61L, Q61V, Q61H and A146T in NRAS. In some examples, only mutations at V600 of BRAF are assessed, and only mutations at G12, G13 and Q61 or NRAS are assessed.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T”, is complementary to the sequence “T-C-A.” Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The term “correlating” refers to determining a relationship between one type of data with another or with a condition or clinical state (e.g., responsiveness or non-responsiveness to cancer therapy).

As used herein, the phrase “correlates with that of a healthy individual” with reference to serum LDH levels, abundance or concentration means that the level, abundance or concentration detected in a test sample (e.g. a serum or blood sample from a patient with cancer) is about the same, or is in the same range, as that considered to be a normal level or range for a healthy individual of the same age. Conversely, the phrase “is increased relative to that of a healthy individual” with reference to serum LDH levels or abundance means that the level, abundance or concentration detected in a test sample (e.g. a serum or blood sample from a patient with cancer) is increased (e.g. by at least or about 10%, 20%, 30%, 40%, 50%, 60%, 70%, 80%, 90%, 100%, 150%, 200% or more) compared to that which is considered to be a normal level for a healthy individual of the same age. Serum LDH is routinely assessed for the diagnosis and treatment of liver diseases, cardiac diseases and tumors of the lung or kidney. Typically, LDH levels are assessed in an enzymatic assay that assesses LDH activity. For example, the assay may involve LDH catalyzing the oxidation of L-Lactate to pyruvate with the concurrent reduction of β-Nicotinamide Adenine Dinucleotide (NAD) to β-Nicotinamide Adenine Dinucleotide (reduced form) (NADH). The rate of change in absorbance at 340 nm over a fixed-time interval is monitored, where the rate of change in absorbance is directly proportional to the activity of LD in the sample. As would be appreciated, normal serum LDH levels or ranges for healthy individuals of any given age or age range can be determined using a group or population of healthy subjects of a that age or age range. Exemplary normal ranges include: 0-5 years old: 140-304 IU/L; 5-10 years old: 142-290 IU/L; 10-15 years old: 115-257 IU/L; >15 years old: 93-198 IU/L.

As used herein, the terms “diagnosis,” “diagnosing” and the like are used interchangeably herein to encompass determining the likelihood that a subject will develop or has a condition or clinical state (e.g., responsiveness or non-responsiveness to cancer therapy). These terms also encompass, for example, determining the level of clinical state (e.g. the level of responsiveness to cancer therapy), as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or derived biomarker values actually has a condition or clinical state (or not) based on a given mathematical model. An increased likelihood for example may be relative or absolute and may be expressed qualitatively or quantitatively. For instance, an increased likelihood may be determined simply by determining the subject's measured or derived biomarker values for one or more cancer therapy biomarkers and placing the subject in an “increased likelihood” category, based upon previous population studies. The term “likelihood” is also used interchangeably herein with the term “probability”.

The term “gene”, as used herein, refers to a stretch of nucleic acid that codes for a polypeptide or for an RNA chain that has a function. While it is the exon region of a gene that is transcribed to form mRNA, the term “gene” also includes regulatory regions such as promoters and enhancers that govern expression of the exon region.

The term “indicator” as used herein refers to a result or representation of a result, including any information, number, ratio, signal, sign, mark, or note by which a skilled artisan can estimate and/or determine a likelihood of whether or not a subject suffering from a cancer will respond to a cancer therapy. In the case of the present invention, the “indicator” may optionally be used together with other clinical characteristics to arrive at a determination that the subject is or is not likely to respond to cancer therapy. That such an indicator is “determined” is not meant to imply that the indicator is 100% accurate. The skilled clinician may use the indicator together with other clinical indicia to arrive at a conclusion.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physicochemical interactions typically occur in interactions between molecules. In particular embodiments, all that is required is that the molecules (e.g., nucleic acids or polypeptides) remain immobilized or attached to a support under conditions in which it is intended to use the support, for example in applications requiring nucleic acid amplification and/or sequencing or in in antibody-binding assays. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

As used herein, the term “label” and grammatical equivalents thereof, refer to any atom or molecule that can be used to provide a detectable and/or quantifiable signal. In particular, the label can be attached, directly or indirectly, to a nucleic acid or protein. Suitable labels that can be attached include, but are not limited to, radioisotopes, fluorophores, quenchers, chromophores, mass labels, electron dense particles, magnetic particles, spin labels, molecules that emit chemiluminescence, electrochemically active molecules, enzymes, cofactors, and enzyme substrates. A label can include an atom or molecule capable of producing a visually detectable signal when reacted with an enzyme. In some embodiments, the label is a “direct” label which is capable of spontaneously producing a detectable signal without the addition of ancillary reagents and is detected by visual means without the aid of instruments. For example, colloidal gold particles can be used as the label. Many labels are well known to those skilled in the art. In specific embodiments, the label is other than a naturally-occurring nucleoside. The term “label” also refers to an agent that has been artificially added, linked or attached via chemical manipulation to a molecule.

The “level”, “abundance” or “amount” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment. In specific embodiments in which the level of a biomarker is “reduced” relative to a reference or control, the reduced level may refer to an overall reduction of any of at least about 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced level refers to a decrease in level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the level/amount of the respective biomarker in a reference sample, reference cell, reference tissue, control sample, control cell, or control tissue. In certain embodiments in which the level of a biomarker is “about the same” a reference or control, the level of biomarker varies by less than about 10%, 9%, 8%, 7%, 6%, 5%, 4%, 3%, 2%, 1%, 0.5%, 0.1%, or even less, as compared to the level of biomarker (e.g., protein or nucleic acid (e.g., mRNA or cDNA)), detected by standard art known methods such as those described herein, in a reference sample, reference cell, reference tissue, control sample, control cell, or control tissue.

The term “nucleic acid” or “polynucleotide” as used herein includes RNA, mRNA, miRNA, cRNA, cDNA, mtDNA, or DNA. The term typically refers to a polymeric form of nucleotides of at least 10 bases in length, either ribonucleotides or deoxynucleotides or a modified form of either type of nucleotide. The term includes single and double stranded forms of DNA or RNA.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

The terms “MAP1LC3B expression” (or “LC3B expression”) and “EHMT2 expression” (or “G9a expression”) refers to the transcription and/or translation and/or activity of MAP1LC3B (or LC3B) and EHMT2 (or G9a) respectively. Several methods can be utilized to determine the level of expression, as described in detail below.

As used herein, the term “positive response” means that the result of a treatment regimen includes some clinically significant benefit, such as the prevention, or reduction of severity, of symptoms, or a slowing of the progression of the condition. For example, a reduction in tumor size or tumor burden, or a slowing in the rate of tumor growth or spread (i.e. metastasizing), can indicate a positive response. By contrast, the term “negative response” or “non-response” means that a treatment regimen provides no or minimal clinically significant benefit, such as the prevention, or reduction of severity, of symptoms, or increases the rate of progression of the condition. In some instances, the Response Evaluation Criteria in Solid Tumors (RECIST) is used to assess positive or negative response to therapy (Eisenhauer et al. (2009) Eur J Cancer. 45:228-47; and http://www.irrecist.com/recist/). A positive response may include a “partial response” or “complete response”, such as defined by RECIST 1.1, while a negative response may be equivalent to “stable disease”.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

“Protein,” “polypeptide” and “peptide” are used interchangeably herein to refer to a polymer of amino acid residues and to variants and synthetic analogues of the same.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease (e.g., response to therapy). The skilled artisan will understand that the term “prognosis” refers to an increased probability that a certain course or outcome will occur; that is, that a course or outcome is more likely to occur in a subject exhibiting a given condition, when compared to those individuals not exhibiting the condition.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., faeces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. In specific embodiments, the sample comprises cancer or tumor cells.

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

The terms “subject”, “individual” and “patient” are used interchangeably herein to refer to an animal subject, particularly a vertebrate subject, and even more particularly a mammalian subject. Suitable vertebrate animals that fall within the scope of the invention include, but are not restricted to, any member of the phylum Chordata, subphylum vertebrata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards, etc.), and fish. A preferred subject is a primate (e.g., a human, ape, monkey, chimpanzee). The subject suitably has cancer.

As used herein, the term “treatment regimen” or “therapy” refers to prophylactic and/or therapeutic (i.e., after onset of a specified condition) treatments, unless the context specifically indicates otherwise. The terms encompasses natural substances and pharmaceutical agents (i.e., “drugs”) as well as any other treatment regimen including but not limited to dietary treatments, physical therapy or exercise regimens, surgical interventions, and combinations thereof.

It will be appreciated that the terms used herein and associated definitions are used for the purpose of explanation only and are not intended to be limiting.

### 2. Cancer Therapy Biomarkers and Their Use

The present invention concerns methods, compositions, solid supports and kits for assessing the likelihood of a subject with cancer responding to a cancer therapy, and in particular therapy with an immune checkpoint inhibitor. Thus, the methods, compositions, apparatus and kits can be used to stratify subjects into those who are likely to respond to cancer therapy and those who are unlikely to respond to cancer therapy.

2.1 Cancer Therapy Biomarkers

The present inventors have determined that the level or abundance of certain biomarkers (cancer therapy biomarkers) is indicative of the likely responsiveness of a subject with cancer to cancer therapy, and in particular cancer therapy using an immune checkpoint inhibitor. The results presented herein provide clear evidence that a unique biologically-relevant biomarker profile predicts whether a subject is likely to respond to cancer therapy. The methods, compositions, supports and kits of the invention are therefore useful for informing better treatment interventions for subjects with cancer. In this regard, it is proposed that the methods, compositions, supports and kits disclosed herein that are based on these biomarkers may serve in the point-of-care diagnostics that allow for rapid and inexpensive determination of the likely responsiveness of a subject with cancer to cancer therapy, which may result in significant cost savings to the medical system as subjects with cancer can be exposed to therapeutic agents that are likely to be effective. Moreover, and as described herein in more detail, the present inventors have identified interventions that can be used to increase the likely responsiveness of a subject with cancer to cancer therapy, thereby resulting in improved efficacy of therapy in a given subject with cancer, and/or extending the proportion of subjects that respond to therapy.

Cancer therapy biomarkers of the present disclosure include MAP1LC3B, and more particularly the expression products of MAP1LC3B. The MAP1LC3B gene encodes Microtubule Associated Protein 1 Light Chain 3 Beta (MAP1LC3B), a protein involved in the autophagic pathway and which is also referred to herein and in the art as simply LC3B. Expression from the human MAP1LC3B gene results in the transcript set forth in SEQ ID NO:1 (represented as cDNA) and the MAP1LC3B protein set forth in SEQ ID NO:2.

As demonstrated herein, MAP1LC3B can be used as a cancer therapy biomarker to predict whether a subject is likely to respond to cancer therapy, and in particular therapy using an immune checkpoint inhibitor. Expression products of MAP1LC3B, including polynucleotide (e.g. mRNA) and polypeptide expression products of MAP1LC3B, correlate with a subject's responsiveness to cancer therapy and can be assessed so as to predict whether a subject is likely to respond or not respond to cancer therapy, and in particular therapy using an immune checkpoint inhibitor. Thus, in specific embodiments the MAP1LC3B biomarker may be used either by itself or in combination with one or more other cancer therapy biomarkers for the determination of the indicator for assessing the likelihood of a subject responding to cancer therapy.

The present inventors have also determined that other cancer therapy biomarkers have strong diagnostic performance when used in combination with the MAP1LC3B biomarker. In one example, it has been determined that EHMT2 can be used in combination with MAP1LC3B for the determination of the indicator for assessing the likelihood of a subject responding to cancer therapy.

EHMT2 encodes euchromatic histone-lysine methyltransferase 2 (EHMT2), also known and referred to herein as G9a. Expression from the human EHMT2 gene can result in a transcript with the sequence set forth in any one of SEQ ID NOs: 3, 5, 7, 9 and 11 (represented as cDNA) and the EHMT2 protein set forth in any one of SEQ ID NOs: 4, 6, 8, 10, and 12. EHMT2 is one of a larger family of enzymes that can methylate histone H3 lysine 9 (H3K9) from an unmodified state to a dimethylated state (H3K9me2). Dimethylation of H3K9 is correlated with gene repression and is used as a marker of genes silenced epigenetically. Elevated levels of EHMT2 expression have been observed in many types of human cancers, and EHMT2 knockdown has been shown to inhibit the proliferation of cancer cell lines. As demonstrated herein, EHMT2 directly regulates MAP1LC3B in tumor cells via histone methylation, repressing expression of the gene. Inhibition of EHMT2 reverses this repression and increases autophagy (see Examples below).

Notably, when combined with MAP1LC3B, EHMT2 can be used as a cancer therapy biomarker to predict whether a subject is likely to respond to cancer therapy, and in particular therapy using an immune checkpoint inhibitor. As taught herein, expression products of both MAP1LC3B and EHMT2 correlate with a subject's responsiveness to cancer therapy and can be assessed so as to predict whether a subject is likely to respond or not respond to cancer therapy, and in particular therapy using an immune checkpoint inhibitor.

In another example, it has been determined that serum lactate dehydrogenase (LDH) can be used in combination with MAP1LC3B and the BRAF/NRAS mutation status for the determination of the indicator for assessing the likelihood of a subject responding to cancer therapy.

LDH is an established, independent prognostic factor for melanoma survival (Agarwala et al., 2009, Eur J Cancer 45: 1807-1814; Weide et al, 2012, Br J Cancer 107: 422-428; Kelderman et al., 2014, Cancer Immunol Immunother 63: 449-458) and a part of the American Joint Committee on Cancer classification for stage IV melanoma (Balch et al, 2009, J Clin Oncol 27: 6199-6206). Similarly, mutations in BRAF and NRAS are also known to be associated with various cancers (see e.g. Carlino et al. 2014, Br J Cancer 111(2):292-9; Heppt et al. 2017, BMC Cancer 17(1):536). Exemplary mutations that have been associated with cancer include, but are not limited to, G7S, H57Y, F294L, 5365L, G464E, 5465Y, G466E, G469E, L496V, A497V, N581S, N581Y, L584S, D594G, L597S, A598A, V600E, V600K, V600R, K601E, V624F and T740A in BRAF, and G125, G12D, G12A, G13R, G13V, A59D, Q61K, Q61R, Q61L, Q61V, Q61H, A146T (Garman et al. 2017, Cell Reports 21, 1936-1952; Heppt et al., 2017, BMC Cancer. 17: 536).

Thus, specific gene expression products and mutation status are disclosed herein as cancer therapy biomarkers that provide a means for determining the whether a subject is likely to respond to cancer therapy. Evaluation of these cancer therapy biomarkers through analysis of their levels and/or presence in a subject with cancer, or in a sample obtained from a subject with cancer, provides a measured or derived biomarker value for each biomarker for determining an indicator that can be used for assessing the likelihood of a subject responding to cancer therapy.

Evaluation of the cancer therapy biomarkers can be direct (e.g. by directly measuring the levels, amounts or activity of the expression products of MAP1LC3B or EHMT2; the levels, amounts or activity of serum LDH, or the presence of mutations in the BRAF and NRAS genes), or may be indirect. For example, EHMT1 (euchromatic histone-lysine methyltransferase 1; also known as GLP) is a histone methyltransferase encoded by the EHMT1 gene. The EHMT1 gene expresses a transcript with the sequence set forth in SEQ ID NO: 25 (represented as cDNA) and the EHMT1 protein set forth in SEQ ID NO: 26. EHMT1 and EHMT2 assemble into a heterodimeric complex in a stoichiometric 1:1 ratio and function together to catalyze H3K9 dimethylation (see e.g. Tachibana et al. (2005) Genes Dev, 19:815-26). Thus, EHMT1 can be used as a surrogate for EHMT2, whereby the levels or activity of EHMT2 can be assessed indirectly by assessing the levels or activity of an expression product of EHMT1, given that EHMT1 and EHMT2 function as a heterodimer (in a 1:1 ratio) to regulate MAP1LC3B in tumor cells via histone methylation.

2.2 Sample Preparation

Generally, a sample from a subject with cancer is processed prior to cancer therapy biomarker detection or quantification. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide analysis (e.g., quantification of RNA or protein biomarkers) from raw sample (e.g., biological fluid such as blood, serum, etc.) without or with limited processing. In some examples, whole cells or tissue sections are isolated and analysed for cancer therapy biomarker expression, such as using immunohistochemistry (IHC) or flow cytometry.

Methods may comprise steps of homogenizing a sample in a suitable buffer, removal of contaminants and/or assay inhibitors, adding a cancer therapy biomarker capture reagent (e.g., a magnetic bead to which is linked an oligonucleotide complementary to a target cancer therapy biomarker), incubated under conditions that promote the association (e.g., by hybridization) of the target biomarker with the capture reagent to produce a target biomarker:capture reagent complex, incubating the target biomarker:capture complex under target biomarker-release conditions. In some embodiments, multiple cancer therapy biomarkers are isolated in each round of isolation by adding multiple cancer therapy biomarkers capture reagents (e.g., specific to the desired biomarkers) to the solution. For example, multiple cancer therapy biomarker capture reagents, each comprising an oligonucleotide specific for a different target cancer therapy biomarker can be added to the sample for isolation of multiple cancer therapy biomarker. It is contemplated that the methods encompass multiple experimental designs that vary both in the number of capture steps and in the number of target cancer therapy biomarkers captured in each capture step. In some embodiments, capture reagents are molecules, moieties, substances, or compositions that preferentially (e.g., specifically and selectively) interact with a particular biomarker sought to be isolated, purified, detected, and/or quantified. Any capture reagent having desired binding affinity and/or specificity to the particular cancer therapy biomarker can be used in the present technology.

For example, the capture reagent can be a macromolecule such as a peptide, a protein (e.g., an antibody or other ligand that binds to a cancer therapy biomarker), an oligonucleotide, a nucleic acid (e.g., nucleic acids capable of hybridizing with the cancer therapy biomarkers), oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary polynucleotide.

Any nucleic acids, including single-stranded and double-stranded nucleic acids, that are capable of binding, or specifically binding, to a target cancer therapy biomarker can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, cancer therapy biomarker capture reagents may comprise a functionality to localize, concentrate, aggregate, etc. the capture reagent and thus provide a way to isolate and purify the target cancer therapy biomarker when captured (e.g., bound, hybridized, etc.) to the capture reagent (e.g., when a target:capture reagent complex is formed). For example, in some embodiments the portion of the capture reagent that interacts with the cancer therapy biomarker (e.g., an oligonucleotide) is linked to a solid support (e.g., a bead, surface, resin, column, and the like) that allows manipulation by the user on a macroscopic scale. Often, the solid support allows the use of a mechanical means to isolate and purify the target:capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target cancer therapy biomarker) from the heterogeneous solution.

The cancer therapy biomarkers may be quantified or detected using any suitable technique. In specific embodiments, the cancer therapy biomarkers are quantified using reagents that determine the level, abundance or amount of individual cancer therapy biomarkers, either as isolated biomarker or as expressed in or on a cell. Non-limiting reagents of this type include reagents for use in nucleic acid- and protein-based assays.

In particular examples, when assessing the BRAF/NRAS mutational status, the genomic DNA from a sample is isolated and subjected to sequencing (e.g. next-generation sequencing, pyrosequencing, Sanger sequencing etc., as is well-known to those skilled in the art).

2.3 Evaluation of Biomarker Nucleic Acid

In some embodiments, biomarkers are assessed by determining biomarker nucleic acid transcript levels. In illustrative nucleic acid-based assays, nucleic acid is isolated from cells contained in the biological sample according to standard methodologies (Sambrook, et al., 1989, supra; and Ausubel et al., 1994, supra). The nucleic acid is typically fractionated (e.g., poly A⁺ RNA) or whole cell RNA. Where RNA is used as the subject of detection, it may be desired to convert the RNA to a complementary DNA. In some embodiments, the nucleic acid is amplified by a template-dependent nucleic acid amplification technique. A number of template dependent processes are available to amplify the cancer therapy biomarker sequences present in a given template sample. An exemplary nucleic acid amplification technique is the polymerase chain reaction (referred to as PCR), which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the biomarker sequence. An excess of deoxynucleotide triphosphates are added to a reaction mixture along with a DNA polymerase, e.g., Taq polymerase. If a cognate cancer therapy biomarker sequence is present in a sample, the primers will bind to the biomarker and the polymerase will cause the primers to be extended along the biomarker sequence by adding on nucleotides. By raising and lowering the temperature of the reaction mixture, the extended primers will dissociate from the biomarker to form reaction products, excess primers will bind to the biomarker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art. In specific embodiments in which whole cell RNA is used, cDNA synthesis using whole cell RNA as a sample produces whole cell cDNA.

In certain advantageous embodiments, the template-dependent amplification involves quantification of transcripts in real-time. For example, RNA or DNA may be quantified using the real-time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA. In specific embodiments, multiplexed, tandem PCR (MT-PCR) is employed, which uses a two-step process for gene expression profiling from small quantities of RNA or DNA, as described for example in U.S. Pat. Appl. Pub. No. 20070190540. In the first step, RNA is converted into cDNA and amplified using multiplexed gene specific primers. In the second step each individual gene is quantitated by real time PCR. Real-time PCR is typically performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments of RT-PCR assays, a TAQMAN® probe is used for quantitating nucleic acid. Such assays may use energy transfer (“ET”), such as fluorescence resonance energy transfer (“FRET”), to detect and quantitate the synthesized PCR product. Typically, the TAQMAN® probe comprises a fluorescent label (e.g., a fluorescent dye) coupled to one end (e.g., the 5′-end) and a quencher molecule is coupled to the other end (e.g., the 3′-end), such that the fluorescent label and the quencher are in close proximity, allowing the quencher to suppress the fluorescence signal of the dye via FRET. When a polymerase replicates the chimeric amplicon template to which the fluorescent labeled probe is bound, the 5′-nuclease of the polymerase cleaves the probe, decoupling the fluorescent label and the quencher so that label signal (such as fluorescence) is detected. Signal (such as fluorescence) increases with each PCR cycle proportionally to the amount of probe that is cleaved.

TAQMAN® probes typically comprise a region of contiguous nucleotides having a sequence that is identically present in or complementary to a region of a cancer therapy biomarker polynucleotide such that the probe is specifically hybridizable to the resulting PCR amplicon. In some embodiments, the probe comprises a region of at least 6 contiguous nucleotides having a sequence that is fully complementary to or identically present in a region of a target cancer therapy biomarker polynucleotide, such as comprising a region of at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a target cancer therapy biomarker polynucleotide to be detected and/or quantitated.

In addition to the TAQMAN® assays, other real-time PCR chemistries useful for detecting PCR products in the methods presented herein include, but are not limited to, Molecular Beacons, Scorpion probes and intercalating dyes, such as SYBR Green, EvaGreen, thiazole orange, YO-PRO, TO-PRO, etc. For example, Molecular Beacons, like TAQMAN® probes, use FRET to detect and quantitate a PCR product via a probe having a fluorescent label (e.g., a fluorescent dye) and a quencher attached at the ends of the probe. Unlike TAQMAN® probes, however, Molecular Beacons remain intact during the PCR cycles. Molecular Beacon probes form a stem-loop structure when free in solution, thereby allowing the fluorescent label and quencher to be in close enough proximity to cause fluorescence quenching. When the Molecular Beacon hybridizes to a target, the stem-loop structure is abolished so that the fluorescent label and the quencher become separated in space and the fluorescent label fluoresces. Molecular Beacons are available, e.g., from Gene Link™ (see, www.genelink.com/newsite/products/mbintro.asp).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent label (e.g., a fluorescent dye molecule) is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the fluorescent label on the 5′-end to fluoresce and generate a signal. Scorpion probes are available from, e.g., Premier Biosoft International (see, www.premierbiosoft.com/tech_notes/Scorpion.html).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some embodiments, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction. In some embodiments, multiple target cancer therapy biomarker polynucleotides are detected and/or quantitated in a single multiplex reaction. In some embodiments, each probe that is targeted to a different cancer therapy biomarker polynucleotide is spectrally distinguishable when released from the probe. Thus, each target cancer therapy biomarker polynucleotide is detected by a unique fluorescence signal.

Specific examples of fluorescently labeled ribonucleotides useful in the preparation of real-time PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of real-time PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In certain embodiments, target nucleic acids are quantified using blotting techniques, which are well known to those of skill in the art. Southern blotting involves the use of DNA as a target, whereas Northern blotting involves the use of RNA as a target. Each provides different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above. Following detection/quantification, one may compare the results seen in a given subject with a control reaction or a statistically significant reference group or population of control subjects as defined herein. In this way, it is possible to correlate the amount of cancer therapy biomarker nucleic acid detected with the progression or severity of the disease.

Chip hybridization utilizes biomarker specific oligonucleotides attached to a solid substrate, which may consist of a particulate solid phase such as nylon filters, glass slides or silicon chips (Schena et al. (1995) Science. 270:467-470) designed as a microarray. Microarrays are known in the art and consist of a surface to which probes that correspond in sequence to gene products (such as cDNAs) can be specifically hybridized or bound at a known position for the detection of biomarker gene expression.

Quantification of the hybridization complexes is well known in the art and may be achieved by any one of several approaches. These approaches are generally based on the detection of a label or marker, such as any radioactive, fluorescent, biological or enzymatic tags or labels of standard use in the art. A label can be applied to either the oligonucleotide probes or the RNA derived from the biological sample.

In certain embodiments, the cancer therapy biomarker is a target RNA (e.g., mRNA) or a DNA copy of the target RNA whose level or abundance is measured using at least one nucleic acid probe that hybridizes under at least low, medium, or high stringency conditions to the target RNA or to the DNA copy, wherein the nucleic acid probe comprises at least 15 (e.g., 15, 16, 17, 18, 19, 20, 21, 22, 23, 24, 25, 26, 27, 28, 29, 30, or more) contiguous nucleotides of cancer therapy biomarker polynucleotide. In some embodiments, the measured level or abundance of the target RNA or its DNA copy is normalized to the level or abundance of a reference RNA or a DNA copy of the reference RNA. Suitably, the nucleic acid probe is immobilized on a solid or semi-solid support. In illustrative examples of this type, the nucleic acid probe forms part of a spatial array of nucleic acid probes. In some embodiments, the level of nucleic acid probe that is bound to the target RNA or to the DNA copy is measured by hybridization (e.g., using a nucleic acid array). In other embodiments, the level of nucleic acid probe that is bound to the target RNA or to the DNA copy is measured by nucleic acid amplification (e.g., using a polymerase chain reaction (PCR)). In still other embodiments, the level of nucleic acid probe that is bound to the target RNA or to the DNA copy is measured by nuclease protection assay.

In general, mRNA quantification is suitably effected alongside a calibration curve so as to enable accurate mRNA determination. Furthermore, quantifying transcript(s) originating from a biological sample is preferably effected by comparison to a control sample, which sample is characterized by a known expression pattern of the examined transcript(s).

2.4 Evaluation of Biomarker Proteins

In some embodiments, cancer therapy biomarkers are evaluated at the level of protein expression, either by demonstration of the presence of the protein (either isolated or one or in a cell), or by one or more known functional properties of the biomarker. For example, anti-EHMT2 and anti-MAP1LC3B antibodies for use in EHMT2-specific or MAP1LC3B-specific protein detection are known in the art, are commercially available and can also be readily produced by those skilled in the art. Similarly, anti-LDH antibodies for use in LDH-specific protein detection are commercially available and can also be readily produced by those skilled in the art. The antibodies and the antigen-antibody complexes may be detected by several well-known assays in the art, including immunofluorescence assays, immunohistochemistry, fluorescence activated cell sorting (FACS) analysis, enzyme linked immunosorbent assays (ELISA), radioimmunoassays (RIA), light emission immunoassays and Western blot analysis. in other examples, the activity of the biomarker is assessed. For example, the activity of LDH can be assessed by detecting the amount of NADH produced when LDH converts lactate into pyruvate and NADH.

In particular embodiments, immunofluorescence or immunocytochemistry is performed to detect proteins. Cells, such as tumor cells, may be isolated or enriched by methods known in the art. Isolation or enrichment of cells refers to a process wherein the percentage of certain cells (e.g., tumor cells) is increased relative to the percentage in the sample before the enrichment procedure. Purification is one example of enrichment. For example, circulating tumor cells can be isolated by a first step of CD45⁺ depletion to remove CD45⁺ cells, followed by density gradient centrifugation (see below in Example 6 and Boulding et al. (2018) Scientific Reports 8: 73). In other embodiments, antibodies to surface markers on tumor cells may be attached to a solid support to allow for separation. Procedures for separation may include magnetic separation, using antibody magnetic beads (e.g., Miltenyi™ beads), affinity chromatography, “panning” with antibody attached to a solid matrix or any other convenient technique such as Laser Capture Microdissection. Other techniques providing particularly accurate separation include FACS. Once cells are deposited on slides, they may be fixed, and probed with labeled antibody for detection of a cancer therapy biomarker.

Antibodies specific for a cancer therapy biomarker may be directly conjugated to fluorescent markers, including fluorescein, FITC, rhodamine, Texas Red, Cy3, Cy5, Cy7, and other fluorescent markers, and viewed in a fluorescent microscope, equipped with the appropriate filters. Antibodies may also be conjugated to enzymes, which upon addition of an appropriate substrate commence a reaction providing a colored precipitate over cells with detected biomarker protein. Slides may then be viewed by standard light microscopy. Alternatively, primary antibodies specific for a cancer therapy biomarker may be further bound to secondary antibodies conjugated to the detectable moieties. Cell surface expression can be thus assessed, and the addition of cell permeabilization solutions, such as Triton-X and saponin may be applied to facilitate reagent penetration within cell cytoplasms (“Cell Biology: A Laboratory Handbook”, Volumes 1-111 Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980)).

Immunohistochemistry is quite similar to immunofluorescence or immunocytochemistry, in principle, however tissue specimens are probed with an antibody specific for a cancer therapy biomarker, for example, as opposed to cell suspensions. Biopsy specimens are fixed and processed and optionally embedded in paraffin, sectioned if needed, providing cell or tissue slides subsequently probed with heparanase specific antibodies. Alternatively, frozen tissue may be sectioned on a cryostat, with subsequent antibody probing, obviating fixation-induced antigen masking. Antibodies, as in immunofluorescence or immunocytochemistry, are coupled to a detectable moiety, either fluorescent, or enzyme-linked, and are used to probe tissue sections by methods described for immunofluorescence, and are subsequently visualized by fluorescent or confocal microscopy, depending upon the detection method employed. Visualization of a reaction product precipitate may be viewed by standard light microscopy, if an enzymatic detectable moiety was utilized, following development of the reaction product (“Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980)).

In other embodiments, assays such as ELISA and RIA, which follow similar principles for detection of specific antigens, are used. By way of an illustrative example, MAP1LC3B can be measured using RIA by way of a MAP1LC3B-specific antibody that is radioactively labeled, typically with ¹²⁵I. In ELISA assays a MAP1LC3B-specific antibody is chemically linked to an enzyme. MAP1LC3B-specific capturing antibody is immobilized onto a solid support. Unlabeled specimens, e.g., protein extracts from biological samples are then incubated with the immobilized antibody under conditions where non-specific binding is blocked, and unbound antibody and/or protein removed by washing. Bound MAP1LC3B is detected by a second MAP1LC3B specific labeled antibody. Antibody binding is measured directly in RIA by measuring radioactivity, while in ELISA binding is detected by a reaction converting a colourless substrate into a coloured reaction product, as a function of linked-enzyme activity. Changes can thus readily be detected by spectrophotometry (Janeway C. A. et al. (1997). “Immunobiology” 3.sup.rd Edition, Current Biology Ltd., Garland Publishing Inc.; “Cell Biology: A Laboratory Handbook”, Volumes I-III Cellis, J. E., ed. (1994); “Current Protocols in Immunology” Volumes I-III Coligan J. E., ed. (1994); Stites et al. (eds)). Both assays therefore provide a means of quantification of MAP1LC3B or EHMT2 protein content in a biological sample.

Protein biomarker expression may also be detected via light emission immunoassays. Much like ELISA and RIA, in light emission immunoassays the biological sample/protein extract to be tested is immobilized on a solid support, and probed with a specific label, labeled antibody. The label, in turn, is luminescent, and emits light upon binding, as an indication of specific recognition. Luminescent labels include substances that emit light upon activation by electromagnetic radiation, electro chemical excitation, or chemical activation and may include fluorescent and phosphorescent substances, scintillators, and chemiluminescent substances. The label can be a part of a catalytic reaction system such as enzymes, enzyme fragments, enzyme substrates, enzyme inhibitors, coenzymes, or catalysts; part of a chromogen system such as fluorophores, dyes, chemiluminescers, luminescers, or sensitizers; a dispersible particle that can be non-magnetic or magnetic, a solid support, a liposome, a ligand, a receptor, a hapten radioactive isotope, and so forth (U.S. Pat. Nos. 6,410,696, 4,652,533 and European Patent Application No. 0,345,776), and provide an additional, highly sensitive method for detection of protein expression.

Western blot analysis is another means of assessing cancer therapy biomarker polypeptide content in a biological sample. Protein extracts from biological samples of cells (e.g., tumor cells), are solubilized in a denaturing ionizing environment, and aliquots are applied to polyacrylamide gel matrixes. Proteins separate based on molecular size properties as they migrate toward the anode. Antigens are then transferred to nitrocellulose, PVDF or nylon membranes, followed by membrane blocking to minimize non-specific binding. Membranes are probed with antibodies directly coupled to a detectable moiety, or are subsequently probed with a secondary antibody containing the detectable moiety. Typically, the enzymes horseradish peroxidase or alkaline phosphatase are coupled to the antibodies, and chromogenic or luminescent substrates are used to visualize activity (Harlow E. et al., (1998) Immunoblotting. In Antibodies: A Laboratory Manual, pp. 471-510 CSH Laboratory, cold Spring Harbor, N.Y. and Bronstein I. et al. (1992) Biotechniques 12: 748-753).

In specific embodiments, protein-capture arrays that permit simultaneous detection and/or quantification of a large number of proteins are employed. For example, low-density protein arrays on filter membranes, such as the universal protein array system (Ge, 2000 Nucleic Acids Res. 28(2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, Bio-Rad and Sigma). Various methods for the preparation of antibody arrays have been reported (see, e.g., Lopez et al., 2003 J. Chromatogram. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognize at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors and cell-surface antigens.

Individual spatially distinct protein-capture agents are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample, which is optionally fragmented to form peptide fragments (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second protein in the sample is already known or known to be invariant.

In some embodiments, the cancer therapy biomarker is polypeptide expression product (a target polypeptide) whose level is measured using at least one antigen-binding molecule that is immuno-interactive with the target polypeptide. In these embodiments, the measured level of the target polypeptide is normalized to the level of a reference polypeptide. Suitably, the antigen-binding molecule is immobilized on a solid or semi-solid support. In illustrative examples of this type, the antigen-binding molecule forms part of a spatial array of antigen-binding molecule. In some embodiments, the level of antigen-binding molecule that is bound to the target polypeptide is measured by immunoassay (e.g., using an ELISA).

In other examples, the activity

2.5 Deriving Biomarker Values

Biomarker values can be measured biomarker values, which are values of biomarkers directly measured for the subject, or alternatively could be “derived” biomarker values, which are values that have been derived from one or more measured biomarker values, for example by applying a function to the one or more measured biomarker values. As used herein, biomarkers to which a function has been applied are referred to as “derived bio markers.”

The biomarker values may be determined in any one of a number of ways that are well known in the art. For example, a comprehensive description of biomarker value determination can be found in Intl. Pat. Pub. No. WO 2015/117204, which is incorporated herein by reference in its entirety. In one example, the process of determining biomarker values can include measuring the biomarker values, for example by performing tests on the subject or on sample(s) obtained from the subject.

More typically, however, the step of determining the biomarker values includes having an electronic processing device receive or otherwise obtain biomarker values that have been previously measured or derived. This could include for example, retrieving the biomarker values from a data store such as a remote database, obtaining biomarker values that have been manually input, using an input device, or the like. Suitably, the indicator may be determined using a combination of a plurality of biomarker values, the indicator being at least partially indicative of responsiveness to cancer therapy. Assuming the method is performed using an electronic processing device, an indication of the indicator is optionally displayed or otherwise provided to the user.

In some embodiments, biomarker values are combined, for example by adding, multiplying, subtracting, or dividing biomarker values to determine an indicator value. This step is performed so that multiple biomarker values can be combined into a single indicator value, providing a more useful and straightforward mechanism for allowing the indicator to be interpreted and hence used in determining the likelihood of a subject responding to cancer therapy.

It will be understood that in this context, the biomarkers used within the above-described method can define a biomarker profile for cancer therapy responsiveness, which includes a minimal number of biomarkers (e.g., at least one biomarker), whilst maintaining sufficient performance to allow the biomarker profile to be used in making a clinically relevant determination. Minimizing the number of biomarkers used minimizes the costs associated with performing diagnostic or prognostic tests and in the case of polypeptide biomarkers, allows the test to be performed utilizing relatively straightforward techniques such as quantitative RT-PCR and/or immunofluorescence, and allowing the test to be performed rapidly in a clinical environment. In this regard, the indication provided by the methods described herein could be a graphical or alphanumeric representation of an indicator value. Alternatively, however, the indication could be the result of a comparison of the indicator value to predefined thresholds or ranges, or alternatively could be an indication of the likely responsiveness of a subject to cancer therapy.

Furthermore, producing a single indicator value allows the results of the test to be easily interpreted by a clinician or other medical practitioner, so that test can be used for reliable diagnosis in a clinical environment.

Solely by way of an illustration, the indicator-determining methods suitably include determining at least one biomarker value, wherein the biomarker value is a value measured or derived for at least one cancer therapy biomarker of the subject and is at least partially indicative of a concentration or abundance of the cancer therapy biomarker in a sample taken from the subject, and wherein the at least one cancer therapy biomarker comprises an expression product of MAP1LC3B. In some embodiments, the cancer therapy biomarker profile further comprises an expression product of EHMT2 as a cancer therapy biomarker. In still further embodiments, the level or amount of the expression product of EHMT2 is determined indirectly by measuring the level or amount of the expression product of EHMT1. In some embodiments, the cancer therapy biomarker profile further comprises an expression product of EHMT2 as a cancer therapy biomarker. In alternative embodiments, the at least one cancer therapy biomarker comprises an expression product of MAP1LC3B, serum LDH and BRAF/NRAS mutation status.

The derived biomarker value is then used to determine the indicator for use in determining the likelihood of a subject responding to cancer therapy, either by using the derived biomarker value as an indicator value, or by performing additional processing, such as comparing the derived biomarker value to a reference or the like, as generally known in the art and as described in more detail below, or to another biomarker value. In some embodiments, the indicator is indicative of a level, concentration or abundance of an expression product of MAP1LC3B. In other embodiments, the indicator is indicative of a level or abundance of an expression product of MAP1LC3B and a level, concentration or abundance of an expression product of EHMT2. In a particular embodiment, the indicator is indicative of a ratio of the abundance or concentration of an expression product of MAP1LC3B to the abundance or concentration of an expression product of EHMT2. In further embodiments, the indicator is indicative of a level or abundance of an expression product of MAP1LC3B, a level or abundance of serum LDH and the presence or absence of mutations in BRAF and NRAS.

The derived biomarker values could be combined using a combining function such as an additive model; a linear model; a support vector machine; a neural network model; a random forest model; a regression model; a genetic algorithm; an annealing algorithm; a weighted sum; a nearest neighbor model; and a probabilistic model. In some embodiments, the indicator is compared to an indicator reference, with a likelihood of responsiveness to cancer being determined in accordance with results of the comparison. The indicator reference may be derived from indicators determined for a number of individuals in a reference population. The reference population typically includes individuals having different characteristics, such as a plurality of individuals of different sexes; and/or ethnicities, with different groups being defined based on different characteristics, with the subject's indicator being compared to indicator references derived from individuals with similar characteristics. The reference population can include a plurality of individuals known to be responsive to cancer therapy (including completely responsive and/or partially responsive), and in particular therapy using an immune checkpoint inhibitor; or a plurality of individuals known to be non-responsive to cancer therapy, and in particular therapy using an immune checkpoint inhibitor.

In specific embodiments, the indicator-determining methods of the present invention are performed using at least one electronic processing device, such as a suitably programmed computer system or the like. In this case, the electronic processing device typically obtains at least one measured biomarker value, either by receiving this from a measuring or other quantifying device, or by retrieving these from a database or the like. The processing device then determines the indicator by any suitable means, for example, by calculating a value that is indicative of a ratio of concentrations or amounts of an expression product of MAP1LC3B and concentrations or amounts of an expression product of EHMT2. In one aspect, the present invention encompasses an apparatus comprising such electronic processing device(s).

The processing device can then generate a representation of the indicator, for example by generating a sign or alphanumeric indication of the indicator, a graphical indication of a comparison of the indicator to one or more indicator references or an alphanumeric indication of the likely responsiveness of the subject to the cancer therapy.

The indicator-determining methods of the present invention typically include obtaining a sample from a subject who has been diagnosed with cancer, wherein the sample includes one or more cancer therapy biomarkers (e.g., an expression product of MAP1LC3B and/or EHMT2) and quantifying or otherwise assessing at least one of the biomarkers within the sample to determine biomarker values. This can be achieved using any suitable technique, and will depend on the nature of the biomarker, as described above. Suitably, an individual measured or biomarker value corresponds to the level, abundance or concentration of a cancer therapy biomarker or to a function that is applied to that level or amount. For example, if the indicator in some embodiments of the indicator-determining method of the present invention, which uses a plurality of cancer therapy biomarkers, is based on a ratio of concentrations of two polynucleotides or two polypeptides, this process would typically include quantifying the polynucleotides or polypeptides by any means known in the art, including quantitative RT-PCR or immunofluorescence, or by a functional assay.

In some embodiments, the likelihood of a subject responding to cancer is established by determining one or more cancer therapy biomarker values, wherein an individual cancer therapy biomarker value is indicative of a value measured or derived for a cancer therapy biomarker in a subject or in a sample obtained from the subject. These biomarkers are referred to herein as “sample cancer therapy biomarkers.” In accordance with the present invention, a sample cancer therapy biomarker will correspond to a reference cancer therapy biomarker (also referred to herein as a “corresponding cancer therapy biomarker”). By “corresponding cancer therapy biomarker” is meant a cancer therapy biomarker that is structurally and/or functionally similar to a reference cancer therapy biomarker as set forth for example in SEQ ID NOs:1 and 2 (MAP1LC3B transcript and MAP1LC3B protein); SEQ ID NOs: 3-12 (EHMT2 transcripts and EHMT2 proteins); and SEQ ID NOs:27 and 28. (LDH protein; subunit A and B, respectively). Representative corresponding cancer therapy biomarkers include expression products of allelic variants (same locus), homologues (different locus), and orthologues (different organism) of reference cancer therapy biomarker genes. Nucleic acid variants of reference cancer therapy biomarker genes and encoded cancer therapy biomarker polypeptides can contain nucleotide substitutions, deletions, inversions and/or insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference cancer therapy polypeptide.

Corresponding cancer therapy biomarkers include amino acid sequences that display substantial sequence similarity or identity to the amino acid sequence of a reference cancer therapy biomarker polypeptide. In general, an amino acid sequence that corresponds to a reference amino acid sequence will display at least about 80, 81, 82, 83, 84, 85, 86, 97, 88, 89, 90, 91, 92, 93, 94, 95, 96, 97, 98, 99% or even up to 100% sequence similarity or identity to a reference amino acid sequence selected from SEQ ID NOs: 2, 4, 6, 8, 10, 12 27 and 28, as summarized in Table 3. Corresponding cancer therapy biomarkers also include nucleic acid sequences that display substantial sequence similarity or identity to the nucleic acid sequence of a reference cancer therapy biomarker polynucleotide. In general, a nucleic acid sequence that corresponds to a reference nucleic acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to a reference nucleic acid sequence selected from SEQ ID NOs: 3, 5, 7, 9, 11 and 13, as summarized in Table 3.

In some embodiments, calculations of sequence similarity or sequence identity between sequences are performed as follows:

To determine the percentage identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percentage identity between the two sequences is a function of the number of identical amino acid residues shared by the sequences at individual positions, taking into account the number of gaps, and the length of each gap, which need to be introduced for optimal alignment of the two sequences. By contrast, the percentage similarity between the two sequences is a function of the number of identical and similar amino acid residues shared by the sequences at individual positions, taking into account the number of gaps, and the length of each gap, which need to be introduced for optimal alignment of the two sequences. For purposes herein, the sequence of a cDNA of a mRNA transcript, and the sequence of the mRNA itself, are deemed to have 100% sequence identity, although it is understood that one molecule is a DNA molecule and thus comprises “T” while the other is a RNA molecule and this comprises U.

The comparison of sequences and determination of percentage identity or percentage similarity between sequences can be accomplished using a mathematical algorithm. In certain embodiments, the percentage identity or similarity between amino acid sequences is determined using the Needleman and Wünsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percentage identity or similarity between amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol., 215: 403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res. 25: 3389-3402). When utilizing BLAST and Gapped BLAST programs, the default parameters of the respective programs (e.g., XBLAST and NBLAST) can be used.

Corresponding cancer therapy biomarker polynucleotides also include nucleic acid sequences that hybridize to reference cancer therapy biomarker polynucleotides, or to their complements, under stringency conditions described below. As used herein, the term “hybridizes under low stringency, medium stringency, high stringency, or very high stringency conditions” describes conditions for hybridization and washing. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a corresponding cancer therapy biomarker polynucleotide is one that hybridizes to a disclosed nucleotide sequence (e.g., any one of SEQ ID NOs: 1, 3, 5, 7, 9 or 11) under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

### 3. Biomarker Detection Kits, Compositions and Supports

All the essential reagents required for detecting and quantifying the cancer therapy biomarkers of the invention may be assembled together in a kit. In some embodiments, the kit comprises a reagent that permits quantification of at least one cancer therapy biomarker. In some embodiments, the kit comprises: (i) at least one reagent that allows quantification (e.g., determining the abundance, concentration or level) of an expression product of MAP1LC3B in a biological sample; and optionally (ii) instructions for using the at least one reagent. In some embodiments, the kit further comprises (iii) at least one reagent that allows quantification of a polynucleotide or polypeptide expression product of EHMT2 or EHMT1 in a biological sample; (iv) at least one reagent that allows quantification of serum LDH in a biological sample; and/or at least one reagent that allows detection of a mutation in NRAS or BRAF.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively, or in addition, the media can contain internet addresses that provide the instructions.

Reagents that allow quantification of a cancer therapy biomarker include compounds or materials, or sets of compounds or materials, which allow quantification of the biomarker. In specific embodiments, the compounds, materials or sets of compounds or materials permit determining the level or abundance of a polypeptide or polynucleotide (i.e., a transcript or protein expressed from MAP1LC3B, EHMT2 or EHMT1 or serum LDH).

The kits may also optionally include appropriate reagents for detection of labels, positive and negative controls, washing solutions, blotting membranes, microtiter plates, dilution buffers and the like. For example, a protein-based detection kit may include (i) at least one cancer therapy biomarker polypeptide (for example, MAP1LC3B polypeptide and optionally an EHMT2 or EHMT1 polypeptide, or LDH, which may be used as a positive control); and (ii) an antibody that binds specifically to the cancer therapy biomarker polypeptide. Alternatively, a nucleic acid-based detection kit may include (i) a cancer therapy biomarker polynucleotide (for example, a MAP1LC3B polynucleotide and optionally an EHMT2 or EHMT1 polynucleotide, which may be used as a positive control); and (ii) a primer or probe that specifically hybridizes to a cancer therapy biomarker polynucleotide (e.g. a cDNA of a MAP1LC3B, EHMT2 or EHMT1 transcript or the transcript itself). Also included may be enzymes suitable for amplifying nucleic acids including various polymerases (reverse transcriptase, Taq, Sequenase™, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

The kit can also feature various devices (e.g., one or more) and reagents (e.g., one or more) for performing one of the assays described herein; and/or printed instructions for using the kit to quantify the expression of a cancer therapy biomarker gene.

The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

Also provided are compositions and solid supports for determining an indicator used in assessing a likelihood of a subject with cancer responding to cancer therapy. The compositions and solid support may be relevant to applications where the cancer therapy biomarker is polypeptide or a polynucleotide.

In one embodiment, the composition contains MAP1LC3B transcript or cDNA thereof, and at least one oligonucleotide primer or probe that hybridizes to the MAP1LC3B transcript or cDNA; and optionally a EHMT2 or EHMT1 transcript or cDNA thereof and at least one oligonucleotide primer or probe that hybridizes to the EHMT2 or EHMT1 transcript or cDNA thereof. As would be appreciated, the level of transcript or cDNA in the composition is reflective of the level of transcript in the subject from which the sample is taken and obtained, and can thus be used in the method of the present invention to assess the likelihood of a subject responding to cancer therapy.

Alternatively, the compositions may comprise a polypeptide expression product of MAP1LC3B and a detection agent that binds to the polypeptide expression product of MAP1LC3B; and optionally a polypeptide expression product of EHMT2 or EHMT1 and a detection agent that binds to the polypeptide expression product of EHMT2 or EHMT1. In particular embodiments, the composition contains tumor cells that comprise the polypeptide expression product of MAP1LC3B and optionally the polypeptide expression product of EHMT2 or EHMT1. Typically, the detection agents are antibodies or antigen-binding fragments thereof that are specific for the polypeptide expression product of MAP1LC3B or the polypeptide expression product of EHMT2 or EHMT1, respectively.

Solid supports of the present invention include those to which at least one oligonucleotide primer or probe that hybridizes to a MAP1LC3B transcript or cDNA thereof, and optionally at least one oligonucleotide primer or probe that hybridizes to a EHMT2 or EHMT1 transcript or cDNA thereof, are immobilized. In some embodiments, a MAP1LC3B transcript or cDNA thereof, and optionally a EHMT2 or EHMT1 transcript or cDNA thereof, is/are hybridized to their respective oligonucleotides or probes.

### 4. Diagnostic and Therapeutic Methods

The indicator determined using the methods of the present invention can be used for assessing a likelihood of a subject responding to cancer therapy, and in particular therapy that includes an immune checkpoint inhibitor. Immune checkpoint inhibitors include, for example, those that target CTLA-4 and thus block or inhibit the interaction between CTLA-4 and CD80/CD86 (i.e. CTLA-4 inhibitors, such as ipilimumab or tremelimumab), those that target PD-1 and thus block or inhibit the interaction between PD-1 and PD-L1 (i.e. PD-1 inhibitors, such as pembrolizumab, pidilizumab, nivolumab, REGN2810, CT-001, AMP-224, BMS-936558, MK-3475, MEDI0680 and PDR001), and those that target PD-L1 and thus block or inhibit the interaction between PD-1 and PD-L1 (i.e. PD-L1 inhibitors such as atezolizumab, durvalumab, avelumab, BMS-936559 and MEDI4736). In some examples, a combination of immune checkpoint inhibitors constitute the cancer therapy.

As established in the Examples below, the level, abundance or concentration of an expression product of MAP1LC3B can be used to predict whether a subject is likely to respond to cancer therapy. For example, the level of expression of MAP1LC3B generally corresponds to responsiveness to therapy, whereby subjects with higher MAP1LC3B expression are typically likely to respond to cancer therapy and subjects with lower MAP1LC3B expression are typically unlikely to respond to cancer therapy (e.g. are likely to be non-responsive to cancer therapy). For example, subjects can be separated into two groups based on their relative level of MAP1LC3B expression: MAP1LC3B-high and MAP1LC3B-low, whereby MAP1LC3B-high subjects are more likely to respond to cancer therapy with an immune checkpoint inhibitor than MAP1LC3B-low subjects.

Thus, provided herein is a method for determining an indicator used in assessing a likelihood of a subject with cancer responding to cancer therapy, the method comprising, consisting or consisting essentially of: (1) determining a biomarker value for at least one cancer therapy biomarker in a sample from the subject, wherein the, or one of the, cancer therapy biomarker(s) is an expression product of MAP1LC3B; and (2) determining the indicator using the biomarker value(s), wherein the indicator is at least partially indicative of the likelihood of responsiveness to cancer therapy; wherein the cancer therapy comprises therapy with an immune checkpoint inhibitor.

As demonstrated herein, where the abundance or concentration of the expression product of MAP1LC3B is increased relative to the abundance or concentration that correlates with a negative response to cancer therapy, or is about the same as the abundance or concentration that correlates with a positive response to cancer therapy, the indicator can be determined to be at least partially indicative of a positive response to therapy, and the subject can therefore be assessed as being likely to exhibit a positive response to therapy. Conversely, where the abundance or concentration of the expression product of MAP1LC3B is decreased relative the abundance or concentration that correlates with a positive response to cancer therapy, or is about the same as the abundance or concentration that correlates with a negative response to cancer therapy, the indicator can be determined to be at least partially indicative of a negative response to therapy, and the subject can thus be assessed as being likely to exhibit a negative response (e.g. a non-response) to therapy.

As also established in the Examples below, the level, abundance or concentration of an expression product of EHMT2 can also be used to predict whether a subject is likely to respond to cancer therapy. Typically, the level of expression of EHMT2 inversely correlates to responsiveness to therapy, whereby subjects with lower EHMT2 expression are generally likely to respond positively to cancer therapy, and subjects with higher EHMT2 expression are generally unlikely to respond positively to cancer therapy (or are likely to be non-responsive to cancer therapy). For example, subjects can be separated into two groups based on their relative level of EHMT2 expression: EHMT2-low and EHMT2-high, whereby EHMT2-low subjects are more likely to respond to cancer therapy with an immune checkpoint inhibitor than EHMT2-high subjects.

Thus, in some embodiments, where the abundance or concentration of the expression product of EHMT2 is increased relative to the abundance or concentration that correlates with a positive response to cancer therapy, or is about the same as the abundance or concentration that correlates with a negative response to cancer therapy, the indicator can be determined to be at least partially indicative of a negative response to therapy and the subject can therefore be assessed as being likely to exhibit a negative response (e.g. a non-response) to therapy. In other embodiments, where the abundance or concentration of the expression product of EHMT2 is about the same as the abundance or concentration that correlates with a positive response to cancer therapy, or is decreased relative to the abundance or concentration that correlates with a negative response to cancer therapy, the indicator can be determined to be at least partially indicative of a positive response to therapy and the subject can therefore be assessed as being likely to exhibit a positive response to therapy.

Notably, the ratio of MAP1LC3B expression to EHMT2 expression is particularly useful as an indicator, or for deriving an indicator, used in assessing a likelihood of a subject with cancer responding to cancer therapy. As demonstrated in Example 6, subjects who demonstrate a positive response (including a complete or partial response) to therapy with an immune checkpoint inhibitor have a ratio of MAP1LC3B expression to EHMT2 expression that is higher than the ratio observed in subjects who do not respond to the therapy. Thus, in some embodiments, where the ratio is higher relative to a ratio that correlates with a negative response to therapy, or is about the same as a ratio that correlates with a positive response to therapy, the indicator can be determined to be at least partially indicative of a positive response to cancer therapy and the subject can thus be assessed as being likely to exhibit a positive response to cancer therapy. Conversely, where the ratio is lower relative to a ratio that correlates with a positive response to therapy, or is about the same as a ratio that correlates with a negative response to therapy, the indicator can be determined to be at least partially indicative of a negative response to therapy and the subject can thus be assessed as being unlikely to exhibit a positive response to therapy (or likely to exhibit a negative response, e.g. a non-response, to therapy).

Moreover, and as also demonstrated below, subjects who exhibit a complete response to cancer therapy with an immune checkpoint inhibitor have a ratio of MAP1LC3B expression to EHMT2 expression that is higher than the ratio observed in subjects who exhibit only a partial response to the therapy. Further sub-classification of subjects as partial- or complete-responders to cancer therapy with an immune checkpoint inhibitor can therefore also be made. Thus, in some embodiments, where the ratio is higher relative to a ratio that correlates with a partial response to therapy, or is about the same as a ratio that correlates with a complete response to therapy, the indicator can be determined to be at least partially indicative of a complete response to cancer therapy and the subject can thus be assessed as being likely to exhibit a complete response to cancer therapy. Conversely, where the ratio is lower relative to a ratio that correlates with a complete response to therapy, or is about the same as a ratio that correlates with a partial response to therapy, the indicator can be determined to be at least partially indicative of a partial response to therapy and the subject can thus be assessed as being likely to exhibit a partial response to therapy. As an extension, where the ratio is lower relative to a ratio that correlates with a partial response to therapy, or is about the same as a ratio that correlates with a negative response to therapy, the indicator can be determined to be at least partially indicative of a negative response to therapy and the subject can thus be assessed as being likely to exhibit a negative response to therapy.

In some examples therefore, subjects can be separated into three groups based on the ratio of MAP1LC3B expression to EHMT2 expression: high, intermediate and low, whereby high ratio subjects are likely to exhibit a complete response, intermediate ratio subjects are likely to exhibit a partial response, and low ratio subjects are likely to exhibit non-response to cancer therapy with an immune checkpoint inhibitor. In some embodiments, a high ratio represents a ratio of from about 1.65 to about 2.00, such as or as about 1.65, 1.70, 1.75, 1.80, 1.85, 1.90, 1.95 or 2.00; an intermediate ratio represents a ratio of from about 1.30 to about 1.60, such as or as about 1.30, 1.35, 1.40, 1.45, 1.50, 1.55 or 1.60; and/or a low ratio represents a ratio of from about 0.50 to about 0.90, such as or as about 0.50, 0.55, 0.60, 0.65, 0.70, 0.75, 0.80, 0.85 or 0.90.

As also established in the Examples below, the level, abundance or concentration of serum LDH and mutation status of BRAF and NRAS can also be used in combination with the level, abundance or concentration of the expression product of MAP1LC3B to predict whether a subject is likely to respond to cancer therapy. Typically, subjects with lower expression of MAP1LC3B are generally unlikely to respond to therapy or are likely to only partially respond to therapy, regardless of their LDH levels and BRAF/NRAS mutation status. Conversely, subjects with higher expression of MAP1LC3B are generally likely to respond to therapy, unless they have increased LDH levels relative to a “normal” or “healthy” level and do have BRAF/NRAS mutations, in which case they are unlikely to respond to cancer therapy. In particular embodiments of this aspect, expression of MAP1LC3B is assessed by determining the percentage of tumor cells in a sample that are positive for LCB3 expression. In further examples, a percentage under, or equal to or under, a particular reference or cutoff indicates that the subject is generally unlikely to respond to cancer therapy, and a percentage equal to or over, or over, a particular cutoff indicates that the subject is generally likely to respond to cancer therapy unless they have increased LDH levels relative to a “normal” or “healthy” level and do have BRAF/NRAS mutations. Such references or cutoffs can be determined empirically by those skilled in the art. Non-limiting examples of such cutoffs include 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5 and 25% of LC3B⁺ cells in a population.

As also demonstrated herein, inhibition of EHMT2 in cancer cells results in increased MAP1LC3B expression and autophagy-mediated cell death. Of note, and as described above and herein, MAP1LC3B expression levels correlate with a positive response to cancer therapy, and in particular therapy with an immune checkpoint inhibitor, while EHMT2 expression levels inversely correlate with a positive response to cancer therapy, and in particular therapy with an immune checkpoint inhibitor. Accordingly, subjects who are unlikely to respond, or a likely to only partially respond, to cancer therapy with an immune checkpoint inhibitor may be candidates for sensitization to that cancer therapy, such as by administering a therapeutic that increases MAP1LC3B expression levels and/or increases LC3B levels. For example, a LC3B polypeptide or a polynucleotide encoding LC3B could be administered to a subject. In other examples, the therapeutic is an EHMT2 inhibitor. Without being bound by theory, it is proposed that the EHMT2 inhibitor will induce or increase MAP1LC3B expression by reducing histone H3K9 methylation, thereby leading to improved responsiveness to cancer therapy. Moreover, because EHMT2 forms a heterodimeric complex with EHMT1 to catalyze dimethylation, an EHMT1 inhibitor can be used in place of, or in addition to, the EHMT2 inhibitor, resulting the same effect of inducing or increasing MAP1LC3B expression by reducing histone H3K9 methylation.

Thus, provided herein are methods for assessing whether a subject with cancer is a candidate for sensitization to cancer therapy with an immune checkpoint inhibitor, wherein a subject is considered to be a candidate for sensitization to cancer therapy with an immune checkpoint inhibitor when the subject has been assessed as being unlikely to respond cancer therapy or likely to only partially respond to cancer therapy in accordance with the methods described herein. In a particular example, the subject is considered to be a candidate for sensitization to cancer therapy with an immune checkpoint inhibitor when the subject has one of the following biomarker profiles: (i) the abundance or concentration of the polynucleotide or polypeptide expression product of MAP1LC3B is decreased relative to a reference level, the abundance or concentration of serum LDH correlates with that of a healthy subject, and the BRAF/NRAS mutation status is negative; (ii) the abundance or concentration of the polynucleotide or polypeptide expression product of MAP1LC3B is increased relative to a reference level, the abundance or concentration of serum LDH is increased relative to that of a healthy subject, and the BRAF/NRAS mutation status is positive; (iii) the abundance or concentration of the polynucleotide or polypeptide expression product of MAP1LC3B is increased relative to a reference level, the abundance or concentration of serum LDH is increased relative to that of a healthy subject, and the BRAF/NRAS mutation status is positive; (iv) the abundance or concentration of the polynucleotide or polypeptide expression product of MAP1LC3B is decreased relative to a reference level, the abundance or concentration of serum LDH is increased relative to that of a healthy subject, and the BRAF/NRAS mutation status is negative or positive; and (v) the abundance or concentration of the polynucleotide or polypeptide expression product of MAP1LC3B is decreased relative to a reference level, the abundance or concentration of serum LDH correlates with that of a healthy subject, and the BRAF/NRAS mutation status is positive.

Also provided are methods for sensitizing a subject to therapy with an immune checkpoint inhibitor, wherein the subject has been assessed as being a candidate for sensitization to therapy with an immune checkpoint inhibitor, and/or has been assessed as being unlikely to respond cancer therapy or likely to only partially respond to cancer therapy in accordance with the methods described herein, by administering a therapeutic that increases MAP1LC3B expression levels and/or increases LC3B levels.

EHMT2 inhibitors are well known in the art and include any which partially or completely inhibit or reduce the level of EHMT2 expression or EHMT2 activity The EHMT2 inhibitor may be specific for EHMT2, or may also act on other molecules, such as other histone methyltransferases (e.g. EHMT1), to inhibit their expression and/or activity. Examples of EHMT2 inhibitors include small molecules, antibodies and antigen-binding fragments thereof, polynucleotides, such as antisense and inhibitory RNA (e.g., siRNA and shRNA) molecules, and other molecules such as zinc finger nucleases. In some embodiments, the inhibitor is a small molecule or an antibody or antigen binding fragment thereof that binds to a EHMT2 polypeptide, such as a polypeptide having a sequence set forth in any one of SEQ ID NOs: 4, 6, 8, 10 or 12 or a polypeptide having at least of about 85%, 86%, 87%, 88%, 89%, 90%, 91%, 92%, 93%, 94%, 95%, 96%, 97%, 98% or 99% sequence identity thereto. In other embodiments the inhibitor is a polynucleotide that is complementary to, and hybridizes to, a EHMT2 polynucleotide, such as a polynucleotide having a sequence set forth in any one of SEQ ID NOs: 3, 5, 7, 9 and 11; a complement thereof; or a polynucleotide having at least of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto. In certain embodiments, the inhibitor reduces or inhibits, the expression level or biological activity of EHMT2 by at least 10%, 20%, 30%, 40%, 50%, 60%, 70%, 80%, 90% or more.

EHMT1 inhibitors are also well known in the art and include any which partially or completely inhibit or reduce the level of EHMT1 expression or EHMT1 activity The EHMT1 inhibitor may be specific for EHMT1, or may also act on other molecules, such as other histone methyltransferases (e.g. EHMT2), to inhibit their expression and/or activity. Examples of EHMT1 inhibitors include small molecules, antibodies and antigen-binding fragments thereof, polynucleotides, such as antisense and inhibitory RNA (e.g., siRNA and shRNA) molecules. In some embodiments, the inhibitor is a small molecule or an antibody or antigen binding fragment thereof that binds to a EHMT1 polypeptide, such as a polypeptide having a sequence set forth in SEQ ID NO: 26 or a polypeptide having at least of about 85%, 86%, 87%, 88%, 89%, 90%, 91%, 92%, 93%, 94%, 95%, 96%, 97%, 98% or 99% sequence identity thereto. In other embodiments the inhibitor is a polynucleotide that is complementary to, and hybridizes to, a EHMT1 polynucleotide, such as a polynucleotide having a sequence set forth in SEQ ID NO: 25; a complement thereof; or a polynucleotide having at least of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto. In certain embodiments, the inhibitor reduces or inhibits, the expression level or biological activity of EHMT1 by at least 10%, 20%, 30%, 40%, 50%, 60%, 70%, 80%, 90% or more.

Methods for assessing the inhibition of the expression level or activity of EHMT2 or EHMT1 by an inhibitor are well known in the art and can be used to identify or select suitable inhibitors for use in accordance with the present invention. For example, expression levels of EHMT2 or EHMT1 before and after exposure of a cell to an inhibitor can be assessed at the transcript or protein level using methods well known in the art, including those described above and in the Examples herein. The ability of EHMT2 or EHMT1 to methylate H3K9 can also be assessed using methods well known in the art, such as those described WO2018005799, WO2017181177, WO2017142947, WO2015200329, WO2015192981, WO2014066435 and United States Patent Publication No. 2015274660. For example, the levels of H3K9me2 in cells following exposure of the cells to an EHMT2 or EHMT1 inhibitor can be assessed using an antibody specific for H3K9me2. In further embodiments, the ability of an EHMT2 or EHMT1 inhibitor to induce or increase expression of MAP1LC3B is assessed as described below.

In particular embodiments, the EHMT2 inhibitor is a small molecule, such as a small molecule that inhibits EHMT2 activity. Exemplary small molecule inhibitors of EHMT2 include, but are not limited to, A-366 (Pappano et al. (2015) PLoS ONE 10(7): e0131716), BIX01294 (Kubicek et al. (2007) Mol Cell 25:473-481), BRD4770 (Yuan et al. (2012) ASC Chem Biol 7:1152-1157), CM-272 (Jose-Eneriz et al. (2017) Nat Comm 8:15424), E72 (Chang et al. (2010) J. Mol. Biol. 400:1-7), UNCO224 (Liu et al. (2009) J. Med. Chem. 52:7950), UNC0321 (Liu et al. (2010) J. Med. Chem. 53:5844-5857), UNC0631 (Liu et al. (2011) J Med Chem. 54:6139-50), UNC0638 (Vedadi et al. (2011) Nat Chem Biol. 2011 Jul. 10; 7(8): 566-574), UNC0642 (Liu et al (2013) J. Med. Chem. 56:8931), UNC0646 (Liu et al. (2011)), verticillin A (Paschall et al. (2015) J Immunol. 195:1868-1882), sinefungin analogues (Devkota et al. (2014) ACS Med Chem Lett. 5(4): 293-297) and any of the EHMT2 inhibitors described in International Patent Publication Nos. WO2018005799, WO2017181177, WO2017142947, WO2015200329, WO2015192981, WO2014066435 and United States Patent Publication No. 2015274660, which are incorporated herein by reference in their entirety.

In other embodiments, the EHMT2 inhibitor is a molecule that reduces EHMT2 mRNA and/or EHMT2 protein levels, such as by inhibiting EHMT2 expression levels. Exemplary of such molecules are inhibitory nucleic acids, including, but not limited to, antisense oligonucleotides (ASOs, including unmodified or modified forms, such as those containing one or more phosphate linkage modifications (e.g. phosphodiester and/or phosphoramidate modifications), sugar modifications (e.g. locked nucleic acid (LNA), 2′-O-methyl (2OMe), S-constrained-ethyl (cEt), 2′-O-methoxy-ethyl (MOE), tricyclo-DNA (Tc-DNA) and/or 2′-Fluoro), and non-ribose modifications (e.g. peptide nucleic acid (PNA) and/or phosphorodiamidate morpholino oligomers PMO), RNAi molecules such as siRNAs and shRNAs (including bi-functional shRNAs), miRNAs and antagomirs (or blockmirs). Antisense or inhibitory nucleic acid molecules comprise a sequence complementary to at least a portion of an RNA transcript of a gene of interest, in this case the EHMT2 transcript (e.g. any set forth in SEQ ID NOs: 3, 5, 7, 9 and 11). The ability to hybridize to the target will depend on both the degree of complementarity and the length of the antisense nucleic acid. Generally, the larger the hybridizing nucleic acid, the more base mismatches with a RNA it may contain and still form a stable duplex (or triplex as the case may be). One skilled in the art can ascertain a tolerable degree of mismatch by use of standard procedures to determine the melting point of the hybridized complex. Polynucleotides that are complementary to the 5′ end of the message, e.g., the 5′ untranslated sequence up to and including the AUG initiation codon, typically work most efficiently at inhibiting translation. However, sequences complementary to the 3′ untranslated sequences of mRNAs have also been shown to be effective at inhibiting translation of mRNAs as well (see generally, Wagner, R., (1994) Nature 372:333-335). Thus, oligonucleotides complementary to either the 5′- or 3′-non-translated, non-coding regions of the gene, could be used in an antisense approach to inhibit translation of endogenous EHMT2 mRNA. Polynucleotides complementary to the 5′ untranslated region of the mRNA should include the complement of the AUG start codon. Antisense polynucleotides complementary to mRNA coding regions are generally less efficient inhibitors of translation but could be used in accordance with the invention. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. In some embodiments, siRNA or shRNA molecules, such as that used in the Examples below, that inhibit EHMT2 expression are used. Such molecules are well known, are commercially available and can be readily produced by skilled artisans. Other molecules that can function as EHMT2 inhibitors include miRNAs that target the EHMT2 gene, such as miR-217 (see e.g. Thienpont et al. (2016) J Clin Invest 127(1):335-348).

EHMT1 inhibitors for use in the present invention include small molecules, such as a small molecules that inhibits EHMT1 activity. Exemplary small molecule inhibitors of EHMT1 include, but are not limited to, A-366 (Pappano et al. (2015) PLoS ONE 10(7): e0131716), BIX01294 (Kubicek et al. (2007) Mol Cell 25:473-481), BRD4770 (Yuan et al. (2012) ASC Chem Biol 7:1152-1157), CM-272 (Jose-Eneriz et al. (2017) Nat Comm 8:15424), E72 (Chang et al. (2010) J. Mol. Biol. 400:1-7), UNCO224 (Liu et al. (2009) J. Med. Chem. 52:7950), UNC0321 (Liu et al. (2010) J. Med. Chem. 53:5844-5857), UNC0631 (Liu et al. (2011) J Med Chem. 54:6139-50), UNC0638 (Vedadi et al. (2011) Nat Chem Biol. 2011 Jul 10; 7(8): 566-574), UNC0642 (Liu et al (2013) J. Med. Chem. 56:8931), UNC0646 (Liu et al. (2011)), verticillin A (Paschall et al. (2015) J Immunol. 195:1868-1882), and any of the EHMT1 inhibitors described in International Patent Publication Nos. WO2017181177, WO2017142947, and United States Patent Publication No. 2015274660, which are incorporated herein by reference in their entirety.

In other embodiments, the EHMT1 inhibitor is a molecule that reduces EHMT1 mRNA and/or EHMT1 protein levels, such as by inhibiting EHMT1 expression levels. Exemplary of such molecules are inhibitory nucleic acids, including, but not limited to, antisense oligonucleotides (ASOs, including unmodified or modified forms, such as those containing one or more phosphate linkage modifications (e.g. phosphodiester and/or phosphoramidate modifications), sugar modifications (e.g. locked nucleic acid (LNA), 2′-O-methyl (2OMe), S-constrained-ethyl (cEt), 2′-O-methoxy-ethyl (MOE), tricyclo-DNA (Tc-DNA) and/or 2′-Fluoro), and non-ribose modifications (e.g. peptide nucleic acid (PNA) and/or phosphorodiamidate morpholino oligomers PMO), RNAi molecules such as siRNAs and shRNAs (including bi-functional shRNAs), miRNAs and antagomirs (or blockmirs). In one example, the EHMT1 inhibitor is an antisense or inhibitory RNA, such as a siRNA or shRNA molecules. siRNA or shRNA molecules, such as that used in the Examples below, that inhibit EHMT1 expression are well known, are commercially available and can be readily produced by skilled artisans. Other molecules that can function as EHMT1 inhibitors include miRNAs that target the EHMT1 gene, such as miR-217 (see e.g. Thienpont et al. (2016) J Clin Invest 127(1):335-348).

The methods of the present invention also extend to the treatment of a subject with a cancer therapy. Thus, also provided is a method for treating cancer in a subject, the method comprising, consisting or consisting essentially of performing the method described above and herein for determining an indicator used in assessing a likelihood of a subject with cancer responding to cancer therapy; and exposing the subject to a cancer therapy on the basis that the indicator is at least partially indicative of a positive response to cancer therapy, wherein the cancer therapy comprises therapy with an immune checkpoint inhibitor. In other embodiments, the indicator is at least partially indicative of a negative response to cancer therapy and the subject is exposed to an EHMT2 or EHMT1 inhibitor to sensitize the subject to cancer therapy, as described above, before the subject is then exposed to the cancer therapy. Thus, also provided is a method for treating a subject with cancer, the method comprising, consisting or consisting essentially of performing the method described above and herein for determining an indicator used in assessing a likelihood of a subject with cancer responding to cancer therapy; administering an EHMT2 or EHMT1 inhibitor to the subject on the basis that the indicator is at least partially indicative of a negative response to cancer therapy, thereby sensitizing the subject to cancer therapy; and exposing the subject to the cancer therapy, wherein the cancer therapy comprises therapy with an immune checkpoint inhibitor.

The cancer therapy includes therapy with at least one immune checkpoint inhibitor, and optionally two or more immune checkpoint inhibitors. Immune checkpoint inhibitors for inclusion in the cancer therapy include, for example, those that target CTLA-4 and thus block or inhibit the interaction between CTLA-4 and CD80/CD86 (i.e. CTLA-4 inhibitors, such as ipilimumab or tremelimumab), those that target PD-1 and thus block or inhibit the interaction between PD-1 and PD-L1 (i.e. PD-1 inhibitors, such as pembrolizumab, pidilizumab, nivolumab, REGN2810, CT-001, AMP-224, BMS-936558, MK-3475, MEDI0680 and PDR001) and those that target PD-L1 and thus block or inhibit the interaction between PD-1 and PD-L1 (i.e. PD-L1 inhibitors such as atezolizumab, durvalumab, avelumab, BMS-936559 and MEDI4736).

The cancer therapy to which the subject is exposed may be a combination cancer therapy, which includes exposure of the subject to one or more therapies (e.g. radiotherapies) or chemotherapeutic agents.

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be cytostatic or cytotoxic. Non-limiting examples of chemotherapeutic agents for use in accordance with the methods of the present invention include any one or more of those in the following categories:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; anti-tumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel; and topoisomerase inhibitors (for example epipodophyllotoxins like etoposide and teniposide, amsacrine, topotecan and camptothecin);

(ii) cytostatic agents such as antiestrogens (for example tamoxifen, toremifene, raloxifene, droloxifene and idoxifene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;

(v) anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense; and

(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

(ix) immunotherapy approaches, including for example ex vivo and in vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Typically, the therapeutic agents described herein, including those included in the cancer therapy (e.g. immune checkpoint inhibitor(s), other chemotherapeutic agents and EHMT2 inhibitors), will be administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time, such as a reduction in tumor burden and the like. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment of cancer, the medical practitioner or veterinarian may evaluate severity of any symptom or clinical sign associated with the presence of the cancer. In any event, those of skill in the art may readily determine suitable dosages of the therapeutic agents and suitable treatment regimens without undue experimentation.

The methods of the present invention are relevant to an assessment of a subject with cancer. In some embodiments, the subject has a cancer that is a solid tumor. In other embodiments, the cancer is a blood tumor (i.e., not a solid tumor). Exemplary types of cancer include, but are not limited to, one or more of the cancer types such as primary cancer, metastatic cancer, breast cancer, colon cancer, rectal cancer, lung cancer, oropharyngeal cancer, hypopharyngeal cancer, oesophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningeal cancer, astrocytoma, glioma, glioblastoma, retinoblastoma, neuroma, neuroblastoma, Schwannoma, meningioma, solid tumors arising from hematopoietic malignancies, leukaemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, refractory malignancy, advanced metastatic cancer, solid tumor, metastatic melanoma, prostate cancer, solid tumors, recurrent or persistent ovarian epithelial cancer, fallopian tube cancer, lung cancer, and primary peritoneal cancer.

In order that the invention may be readily understood and put into practical effect, particular preferred embodiments will now be described by way of the following non-limiting examples.

### EXAMPLES

### Example 1

### G9A (EHMT2) Inhibitor Suppresses Melanoma Cell Survival

Overexpression of EHMT2 (G9a) has been observed in different cancers and has been associated with poorer prognosis. Previously, it has been shown that G9a plays an oncogenic role in breast cancer by repressing the expression of genes involved in tumor suppressive functions (Casciello et al. (2017) Proc Natl Acad Sci USA 114, 7077-7082). To determine the role G9a plays in melanoma cell proliferation and survival, melanoma cell lines with different molecular characteristics were used (Table 1). Various different cutaneous/occult primary melanoma cell lines, including BRAF p,V600E mutants (D05, D14 and D20), two NRAS p.Q61L mutants (C006 and C013), two NF1 null mutants (C008, c.586+1G>A and D22, p.R440X) and two triple wild type (A04 and C092) cell lines were used to assess the levels of G9a using immunoblotting.

TABLE 1
Characteristics of melanoma cells lines

| | Cell lines | | | | | | | | |
|---|---|---|---|---|---|---|---|---|---|
| | A04 | C092 | C006 | C013 | C008 | D22 | D05 | D14 | D20 |
| Stage | 4 | 3 | 3 | 3 | 3 | 4 | 4 | 4 | 4 |
| site | non- | occult | non- | non-CSD | occult | n/a | non-CSD | non-CSD | n/a |
| primary | CSD | | CSD | | | | | | |
| BRAF | wt | wt | wt | wt | wt | wt | V600E | V600E | V600E |
| NF1 | wt | likely | likely | wt | splice | R440X | likely wt | likely wt | likely |
| | | wt | wt | | | | | | wt |
| CDKN2A | HD | wt | n/a | wt | wt | wt | LOH + | LOH + | wt |
| | | | | | | | [frameshift] | P114L | |
| FGFR2 | A648T | wt | n/a | wt | wt | R759stop; | wt | wt | n/a |
| | | | | | | D530N | | | |
| MC1R | V60L | wt | wt | R151C | wt | D294H −/− | R151C +/− | wt | wt |
| | +/− | | | +/−; | | | | | |
| | | | | D294H | | | | | |
| | | | | +/− | | | | | |
| MC1R | r/wt | wt/wt | wt/wt | n/a | wt/wt | R/R | R/wt | wt/wt | wt/wt |
| class | | | | | | | | | |
| Nras | wt | wt | Q61L | Q61L | wt | wt | wt | wt | wt |
| p14ARF | HD | n/a | wt | wt | n/a | wt | wt | wt | wt |
| PPP3R2 | wt | wt | n/a | wt | wt | wt | E51K | wt | wt |
| PTEN | wt | wt | n/a | 380G > A; | wt | wt | wt | HD | HD |
| | | | | Gly127Glu | | | | | |
| PTEN | LOH | wt | wt | LOH | HD | n/a | LOH | HD | n/a |
| zygosity | | | | | | | | | |
| p53 | wt | wt | wt | P278S | wt | E287K | wt | G266E | R248Q |
| Rb | wt | wt | wt | R455X | n/a | n/a | wt | wt | n/a |
| TERT (% | wt | wt | wt | −138CC > TT | −146C/T | −124 C/T | −124C/T | −124 T/T | −146 |
| mutant) | | | | | | | | | T/T |
| MEK | wt | wt | wt | wt | wt | P124L | wt | wt | wt |
| BCL2L12 | wt | wt | wt | wt | F17F | wt | F17F | wt | wt |
| RPS27 | wt | wt | wt | 1st base | wt | 1st base | wt | wt | wt |
| | | | | transcript | | transcript | | | |
| | | | | C > T | | C > T | | | |
| MAP3K9 | wt | wt | wt | wt | R160C | wt | P263L | Y646C | wt |
| FLRT2 | wt | n/a | R486Q | wt | n/a | E102K | wt | wt | wt |
| PTPRD | wt | wt | n/a | wt | wt | n/a | wt | G557R | n/a |
| HDAC7 | wt | wt | n/a | wt | wt | n/a | wt | E892K | n/a |
| HDAC9 | wt | wt | n/a | wt | E982A | n/a | wt | wt | n/a |
| NEK10 | wt | wt | n/a | wt | S14L | n/a | wt | wt | n/a |
| ERBB4 | wt | wt | n/a | wt | R1067Q | n/a | wt | wt | n/a |
| MET | wt | T922I | n/a | wt | E75K; | n/a | wt | wt | n/a |
| | | | | | R242K | | | | |

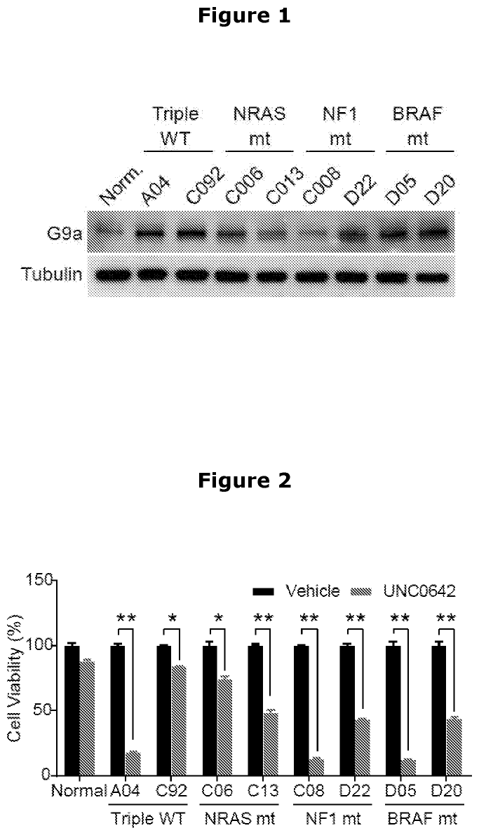

Almost all melanoma cell lines tested expressed a higher level of G9a protein compared to normal melanocytes (FIG. 1).

The role of G9a in cell survival and proliferation was then assessed in vitro using UNC0642, an available small molecule inhibitor of G9a (Liu et al. (2013) Journal of

Medicinal Chemistry 56, 8931-894). Cell survival was significantly attenuated by UNC0642 treatment in the melanoma cell lines, while survival of the normal melanocyte cell culture was unaffected (FIG. 2).

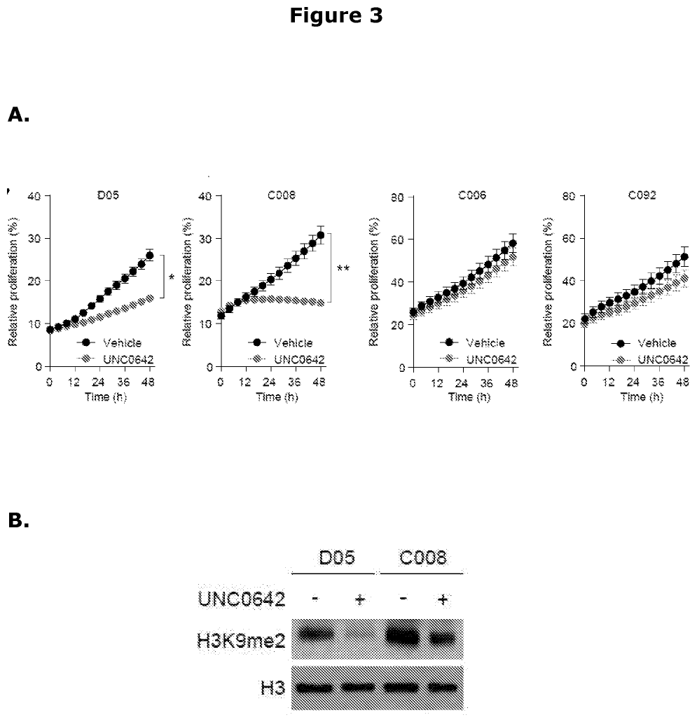

Four cell lines were then chosen for further analysis. These were C006 (NRAS mt), C092 (Triple wt), C008 (NF1 mt) and D05 (BRAF mt). These four lines are representatives of the 4 cutaneous melanoma genomic subtypes and include 2 lines (C008 and D05) that were very sensitive to the inhibitor and 2 lines that were less sensitive (C006 and C092). Proliferation of these lines was evaluated by real time cell imaging using IncuCyte Zoom where cells were grown for 48 hours in the presence or absence of 5 μM UNC0642. G9a inhibition resulted in a significant reduction in proliferation, in the sensitive lines (D05 and C008), compared to vehicle control (data not shown). The number of cells had significantly decreased over 48 hours treatment, indicating that G9a inhibition is actively causing cell death (FIG. 3A). Consistent with proliferation data shown in FIG. 2, C006 and C092 cell lines were much less affected by UNC0642 treatment compared to vehicle. Consistently, a reduction in global H3K9me2 was observed with UNC0642 treatment as early as 8 hours and persisted to 24 hours in the D05 and C008 cell lines (FIG. 3B).

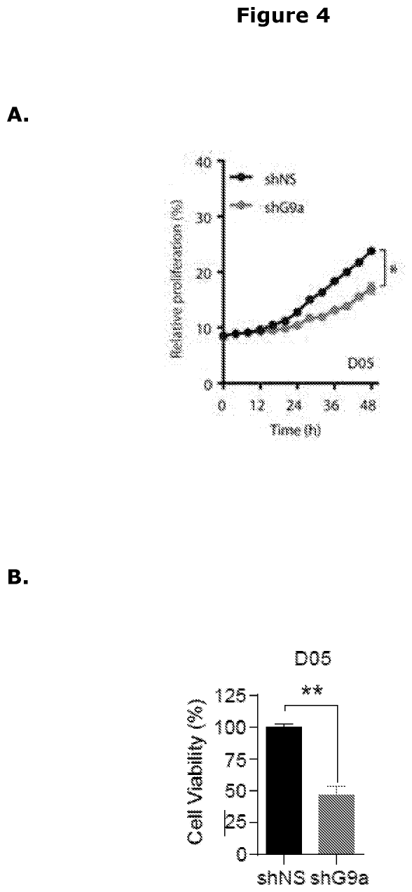

To further investigate the role of G9a, protein expression was reduced by a short hairpin-mediated knock down of G9a in the D05 cell line. This resulted in a similar reduction in proliferation compared to that of UNC0642 treated D05 cells, which was measured using cell viability assays and IncuCyte Zoom (FIGS. 4A and B). G9a inhibition with UNC0642 led to a reduction in cells in both G1 and G2/M phases but a more striking impact was observed in inducing cell death as indicated by greater than 4-fold increase in cells in preG1 phase (data not shown). This increase in preG1 population following G9a inhibitor treatment was absent in C092 (less G9a inhibitor responsive) cell line consistent with cell survival data in FIG. 1B-D (data not shown). Together this data suggest that the loss of histone methyltransferase directly impacts cell survival and indicates that G9a has an important role in maintaining cell proliferation and survival in the melanoma cell lines tested.

### Materials and Methods

Reagents and Cell Cultures

UNC0642 was purchased from Sigma Aldrich. The panel of human melanoma cell lines, derived from cutaneous melanomas, is shown in Table 1 and the cells have all been previously described. Cells were maintained in Roswell Park Memorial Institute (RPMI) 1640 supplemented with 10% fetal bovine serum (FBS), 100 U/mL penicillin, and 100 pg/mL streptomycin in a humidified atmosphere of 5% CO2 at 37° C. Normal melanocytes were grown in Clonetics™ MGM™-4 Melanocyte Growth Medium-4 supplemented with CaCl2, phorbol-12-myristate 13-acetate (PMA), recombinant human fibroblast growth factor basic (rhFGF-B), recombinant human insulin (rh-insulin), hydrocortisone, bovine pituitary extract (BPE), FBS and GA-1000 (30 μg/ml Gentamycin and 15 ng/ml Amphotericin) (Lonza).

Retroviral Transduction

Retroviral constructs expressing short hairpin RNA to G9a (shG9a) or a non-silencing control (shNS) were used as previously described (Casciello et al. (2017) Proc Natl Acad Sci USA 114, 7077-7082). pBABE-puro mCherry-EGFP-LC3B was obtained from Addgene (plasmid #22418) (N'Diaye et al. (2009) EMBO reports 10, 173-179). The viral supernatants were prepared by co-transfecting the constructs with pUMVC3 and pVSV-G into HEK293T cells using Superfect transfection reagent (Qiagen). The supernatants were harvested and used to infect cells in medium, which contained 8 μg/ml polybrene. Cell medium was replaced and fresh growth medium was added after 24 h. Cells were harvested 72 h later or selected using 1 μg/ml of Puromycin sulfate.

IncuCyte Real-Time Imaging and Cell Viability Assays

For proliferation studies, cells (5×10³) were seeded in 96-place wells and allowed to adhere overnight, and incubated in fresh growth medium in the presence of either the G9a inhibitor UNC0642 (Sigma Aldrich) or the vehicle control DMSO (Sigma Aldrich). Proliferation was evaluated via real-time imaging using IncuCyte Zoom (Essen BioScience) and/or by performing a 3-(4,5-dimethylthiazol-2-yl)-2,5-diphenyltetrazolium bromide (MU) assay. After monitoring the cells, 20 μL of MTT (5 mg/mL; Sigma-Aldrich) was added. The plates were incubated at 37° C. for 3 h before the supernatants were removed, and 100 μL of isopropanol was added to each well. The absorbance value (optical density) of each well was measured at 570 nm.

Immunoblotting Analysis

For immunoblotting, protein whole cell lysates were prepared using RIPA lysis buffer containing 20 mM Tris, pH 8.0, 150 mM NaCl, 10% glycerol, 1% Nonidet P-40 containing protease inhibitor cocktail (Roche). Nuclear extracts were obtained using a standard high salt extraction buffer (20 mM HEPES (pH 7.9), 0.32 M NaCl, 1 mM EDTA, and 1 mM EGTA) supplemented with 1mM DU and protease inhibitor cocktail. Protein assays were performed according to the Bradford method using a protein assay kit (BioRad). 20 pg of denatured proteins were separated using 10% sodium dodecyl sulfate-polyacrylamide gel electrophoresis (SDS-PAGE) and transferred to Polyvinylidene difluoride (PVDF) membranes. The membranes were blocked for 1 hour in 5% skim milk in Tris-buffered saline with Tween 20 (TBST; 10 mM Tris-HCl, 150 mM NaCl, 0.1% Tween 20). Immunoblotting was performed with primary anti G9a (#3306, Cell Signaling), H3K9me2 (ab 1220, Abcam), H3 (ab1791, Abcam) or Tubulin (ab6046, Abcam) and detected with either HRP-conjugated anti-rabbit (#7074, Cell Signaling Technology) or HRP-conjugated anti-mouse (#7076, Cell Signaling Technology). After applying ECL detection reagents (GE Healthcare), protein bands were visualized using X-ray film (Fujifilm).

### Example 2

### G9A Inhibition Induces Autophagy-Mediated Cell Death

Having established that G9a inhibition is effective in inducing death of melanoma cells in vitro, the molecular basis for this effect was then investigated. First, cells treated with UNC0642 were examined for its effect on the cleavage of PARP as a measure of apoptosis. No observable PARP cleavage was detected in any cell line treated with UNC0642 (data not shown). Upon examining the effect of UNC0642 on the cell cycle, it was found that neither D05 nor C092 showed any changes in cell cycle status following UNC0642 treatment as determined by flow cytometric analysis (data not shown).

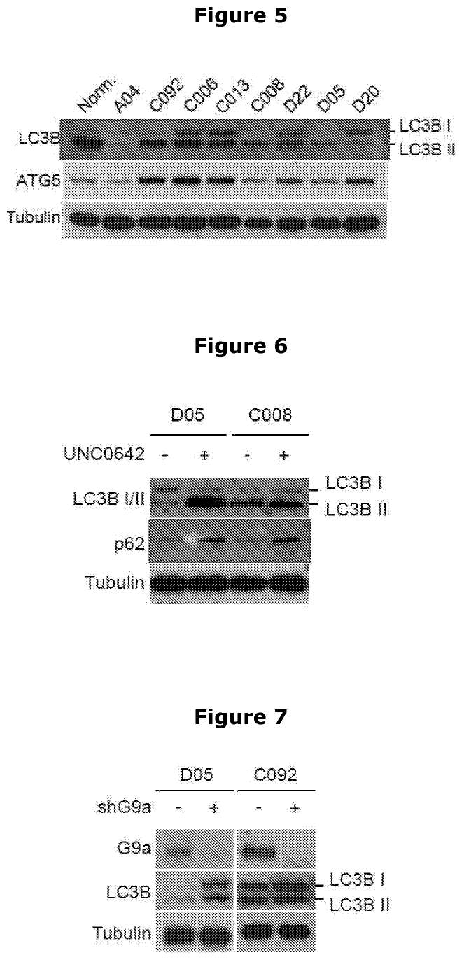

As G9a levels did not correlate with sensitivity to the G9a inhibitor in cell lines that were examined, it was investigated whether the levels of autophagic proteins could be used as a surrogate marker of G9a activity and hence sensitivity to the G9a inhibitor. Therefore, the basal levels of important autophagy related proteins were examined in the melanoma cell lines used in this study. It is has been shown that the inhibition of G9a can cause an induction of the autophagy-related gene 5 (ATG5), Beclin-1 (known also as ATG6) and the conversion of microtubule-associated protein 1 light chain 3 beta (MAP1LC3B or LC3B for short) I (17 KDa) to LCB3 II (15 KDa) through proteolytic cleavage and lipidation (de Narvajas et al. (2013) and Li et a. (2015). These changes are considered a hallmark of mammalian autophagy. When cells undergo autophagy, conversion of a fraction of cytoplasmic LC3B Ito autophagic membrane form LC3B II can be detected by western blot or by immunofluorescence. This conversion correlates with autophagic activity (Kabeya et al (2000) Embo J 19, 5720-5728), therefore the levels of LC3B II can be considered a reliable marker of autophagy. Notably, the basal levels of LC3B II and ATG5 were significantly lower in the D05 and C008 cell lines that were more responsive to G9a inhibition suggesting that the basal level of autophagy might dictate sensitivity to G9a inhibitor (FIG. 5A). To assess whether lowering LC3B levels in the C092 non-responsive cell line would allow increased sensitivity to the G9a inhibitor treatment, cell viability of C092 following G9a inhibitor treatment was assessed. It was observed that cell viability following G9a inhibitor treatment was significantly reduced with LC3B knock down, and that the knock down of LC3B led to a lower level of LC3B II (data not shown).

To further examine the impact of G9a inhibition on autophagy, a pBABE-puro mCherry-EGFP-LC3B construct was used. The distribution of the EGFP-LC3B showed an increase in punctate pattern after treatment with UNC0642 in both the D05 and C092 cell lines with LC3B aggregating at the autophagosomes (data not shown) which was consistent with the immunoblotting results. Cells treated with an inhibitor of autophagy, Bafilomycin A1 (Baf.A1) were used as a control of punctate staining. The levels of LC3B I and II were in the two cell lines responsive to the G9a inhibitor UNC0642 at 5 μM for 8 hours (FIG. 6). LC3B II protein levels dramatically increased in D05 and C008 cell lines following G9a inhibitor treatment. The G9a protein level was transiently reduced using short hairpin-mediated knock down in 2 cutaneous/occult melanoma cell lines (C092 triple wt and D05 BRAF mt) and compared to cells with the methyltransferase activity inhibited using UNC0642. Consistently, LC3B II level increased following G9a knock down in D05 cell line, but not in C092 cell line (FIG. 7).

Together, these results demonstrate the ability of G9a inhibitor to elicit autophagic induction in melanoma cell lines.

### Materials and Methods

Immunoblotting Analysis

Immunoblotting analysis was performed as described above, but also utilizing an anti-LC3B primary antibody (ab48394, Abcam).

Retroviral Transduction

Retroviral transduction was performed as described above.

Immunofluorescence Analysis

Stable cells expressing EGFP-LC3B construct were treated with DMSO, UNC0642 at 5 μM or 20 nM Bafilomycin A1 (Sigma Aldrich) for 8 hours. Images were taken with an EVOS FL auto fluorescent microscope (Invitrogen) and quantitated using ImageJ software.

### Example 3

### Molecular Analysis of MAP1LC3B Regulation

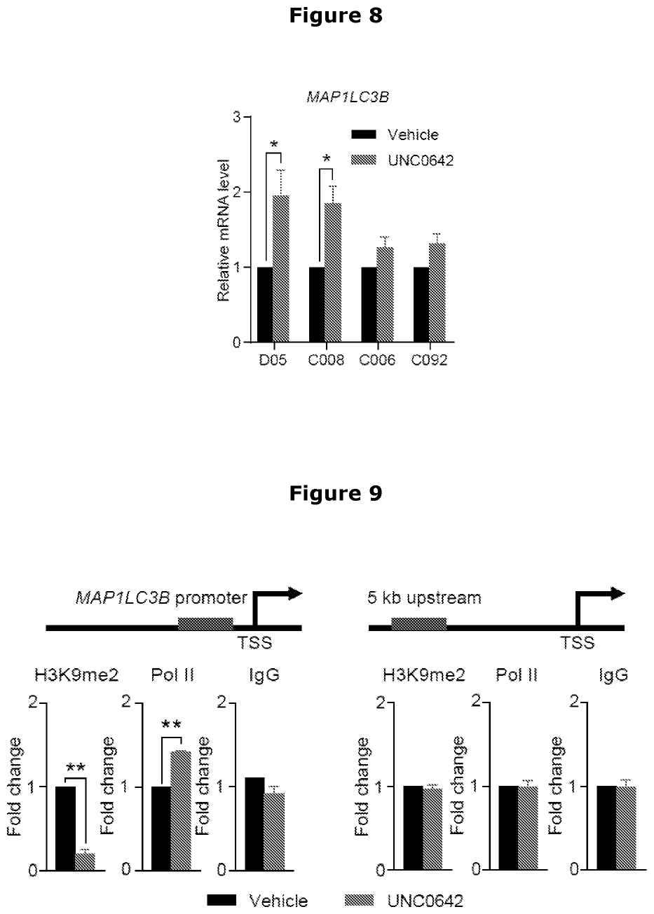

To further examine the impact of G9a on the autophagic processes, the level of MAP1LC3B expression was examined using various molecular techniques. Quantitative real time PCR analysis of the four melanoma cell lines showed that the expression of the MAP1LC3B gene was altered to various degrees in all the cell lines upon UNC0642 treatment (FIG. 8). There was a statistically significant increase in MAP1LC3B expression upon UNC0642 treatment in the very sensitive lines (D05 and C008), with almost 2-fold induction compared to vehicle control (P<0.05). By contrast, the induction of MAP1LC3B expression following UNC0642 was minimal, with no statistically significant difference compared to that of vehicle treatment in the less sensitive cells (C006 and C092) (FIG. 8). Two other important autophagy related genes, BECLIN1 and BNIP3L (data not shown) were also assessed and showed similar significant increases in expression upon UNC0642 treatment.

In order to ascertain whether the inhibition of G9a directly impacts MAP1LC3B expression, chromatin immunoprecipitation (ChIP) was performed on the MAP1LC3B promoter. Changes in histone H3K9 dimethylation (H3K9me2) and acetylation (H3K9Ac), as well as the recruitment of RNA Pol II, were compared between vehicle or UNC0642 treated cells (FIG. 9). Consistent with the increase in gene expression observed in FIG. 8, there was a greater than 4-fold reduction in H3K9me2 concomitant with an increase in RNA Pol II recruitment at the MAP1LC3B promoter following UNC0642 treatment. No change in H3K9me2, or the recruitment of RNA Pol II to the irrelevant region (5 kb upstream of the MAP1LC3B promoter) was observed suggesting a specific regulation of MAP1LC3B by G9a. Together, these results demonstrate that G9a inhibition elicits accumulation of LC3B II in melanoma cell lines by blocking autophagic flux as well as inducing its gene expression via directly demethylating H3K9 in the promoter region.

### Materials and Methods

### Quantitative Real-Time RT-PCR and Chromatin Immunoprecipitation (ChIP) Assays

Quantitative RT-PCR and ChIP assays were conducted as previously described (Lee et al. (2010) Mol Cell 39, 71-85). Briefly, total RNA was isolated from either the tumor cells or from xenografts using Trizol (Invitrogen) and reverse transcription was performed from 2 μg of total RNA using the Superscript III cDNA synthesis system (Invitrogen). The abundance of mRNA was detected by an ABI VIIA7 system with SYBR Green Master Mix (Life Technologies). Primer pairs were designed to amplify 90-150 bp mRNA specific fragments and were confirmed as unique products by melting curve analysis. Sequences of the primers are provided in Table 2. The quantity of mRNA was calculated using the ΔΔCt method and normalized to HPRT. All reactions were performed in triplicates.

### Example 4

### The Effect of G9A Inhibition on Melanoma Growth In Vivo

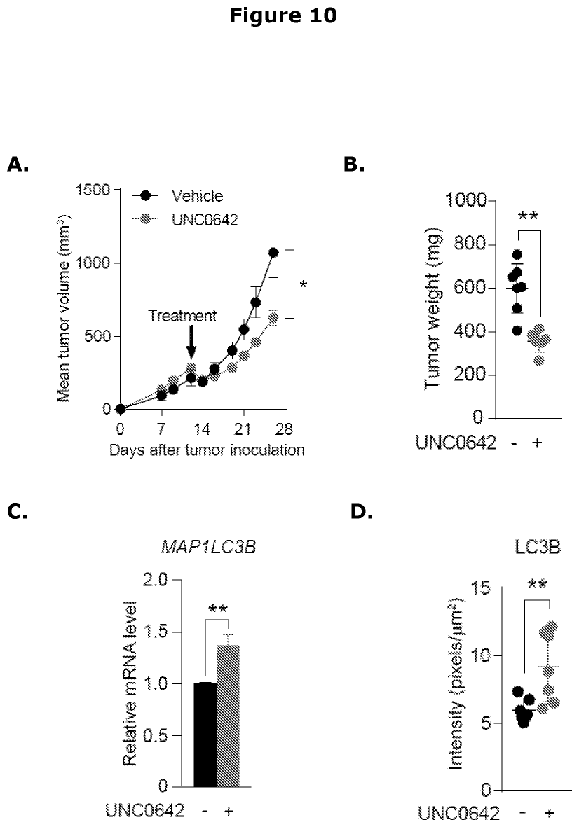

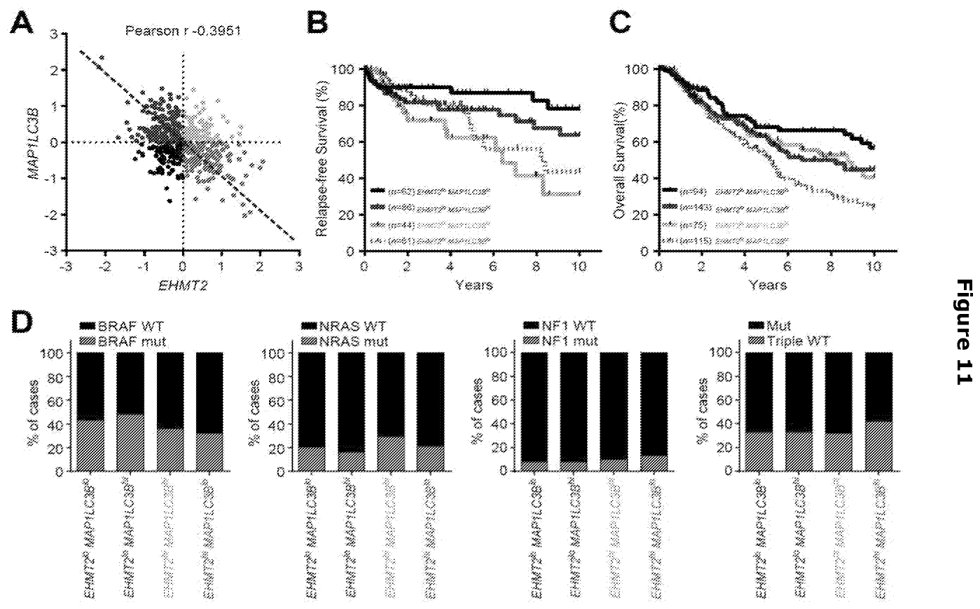

To determine the effect of inhibiting G9a on tumor growth, 6-8 week old SCID mice were subcutaneously injected with D20 BRAF mutant cutaneous melanoma cells. Palpable tumors were allowed to establish (approx. 10 days) before UNC0642 was administered at 5 mg/kg every two days for two weeks. The D20 cell line was used in the subcutaneous model as it had been shown previously to be tumorigenic in vivo and was also sensitive to the drug in vitro. Consistent with the in vitro data where UNC0642 reduced viability of melanoma cells, UNC0642 administration led to statistically significantly reduced tumor growth (FIG. 10A; P<0.05) and tumor weight (FIG. 10B; P<0.01), compared to treatment with vehicle control.

Quantitative real time PCR analysis of RNA extracted from tumors from UNC0642-treated mice also showed a statistically significant increase in expression of the MAP1LC3B gene compared to the vehicle treated group (FIG. 10C; P<0.01). Immunohistochemical analysis of MAP1LC3B in the excised tumors revealed that UNC0642 significantly increased the level of MAP1LC3B compared to vehicle (FIG. 10D). These in vivo results demonstrate that G9a methyltransferase activity has a critical role in regulating tumor growth, and that the in vitro results are recapitulated in vivo.

### Materials and Methods

In Vivo Tumor Growth Analysis

Groups of 8 SCID mice per treatment group were used for xenograft studies to ensure adequate power to detect biological differences. All experiments were approved by the QIMR Berghofer Medical Research Institute Animal Ethics Committee. D20 melanoma cells (2×106) were mixed 1:1 ratio with Growth factor-reduced Matrigel (BD Biosciences) and injected subcutaneously into the flank of 6-8 week old mice in 100 μL volume (day 0) and treatments, either DMSO or UNC0642 at 5 mg/kg were administered as indicated in the figure legends. Tumor volumes (width2×length/2) were measured using a digital calliper, and presented as mean±SD. All animals were sacrificed at the same time and the tumors were dissected for further analysis.

Immunohistochemical Analysis

Tumor sections were fixed in 4% paraformaldehyde. The antibodies used were rabbit monoclonal antibody for G9a (Cell Signaling Technology, 3306S) at 1:300 dilution and goat polyclonal antibody for MAP1LC3B (Santa Cruz Biotechnology, SC-16756) at 1:300 dilution. The universal secondary protocol and the DAB (Biocare Medical) were used to detect and amplify the signal. Aperio ImageScope software was used for imaging and quantitation of 5 non-overlapping tumor regions and evaluating the number of positive pixels per unit area in each region. Empty areas were manually excluded from quantification.

Quantitative RT-PCR

Quantitative RT-PCR was performed as described above.

### Example 5

### MAP1LC3B and G9A Expression in TCGA Melanoma Dataset

The mRNA expression of both G9a (EHMT2) and LC3B (MAP1LC3B) in the TCGA melanoma RNA-seq dataset was next investigated. It was found that their expression was inversely correlated (FIG. 11A), and that the overall and relapse-free survivals of these patients were significantly stratified according to the combined expression of these two genes. As shown in FIG. 11B, patients with high (hi) EHMT2 expression but low (lo) MAP1LC3B expression (EHMT2^{hi}/MAP1LC3B^{lo}) had worse overall survival compared to all other groups. This is in keeping with the inverse outcomes (inactive G9a and high LC3B) when mice or cells were treated with an inhibitor of G9a. For relapse-free (overall) survival (FIG. 11C), EHMT2^{hi}/MAP1LC3B^{lo} patients had worse survival compared to EHMT2^{lo}/MAP1LC3B^{lo} patients (HR 4.39, log rank P=0.001), and EHMT2^{hi}/MAP1LC3B^{hi} patients had worse survival compared to EHMT2^{lo}/MAP1LC3B^{hi} patients (HR 2.04, log rank P=0.036). These data show that patients with low G9a expression did better than those who had high levels of G9a.

It is noteworthy that the four groups based on the expression patterns of G9a and MAP1LC3B did not differ significantly in their driver mutational subtype status (FIG. 11C). The expression of EHMT2 mRNA did not associate with NRAS or NF1 mutations but was higher in BRAF wild type vs. BRAF mutant cases (t-test P=0.0002, data not shown). The expression of MAP1LC3B mRNA did not associate with BRAF, NRAS or NF1 mutation status (data not shown). Multivariate survival analysis was carried out using G9a or MAP1LC3B mRNA expression alone or combined in comparison to other parameters including disease stage, gender and mutational status. The expression of G9a alone was able to stratify patients into different prognostic groups in both overall and relapse-free survival while the expression of MAP1LC3B alone did not (data not shown). Using G9a and MAP1LC3B combined showed a better prognostic indication in terms of overall survival compared to using G9a alone; however, it did not outperform the prognostic indication using disease staging.

### Materials and Methods

In Silico Analysis of Melanoma Global Gene Expression

The melanoma cases in the TCGA dataset were allocated to one of four quartiles based on the expression of EHMT (G9a) and/or MAP1LC3B and the survival of these patients were compared. Overall survival and the relapse-free survival of melanoma patients between tumors with the lowest expression (bottom 25%, quartile 1) were compared to the rest of the tumors. Survival curves were constructed using GraphPad Prism (GraphPad Software), and the log-rank (Mantel-Cox) Test was used for statistical comparisons of survival curves.

### Example 6

### G9A and LC3B as a Therapy Response Marker in Melanoma

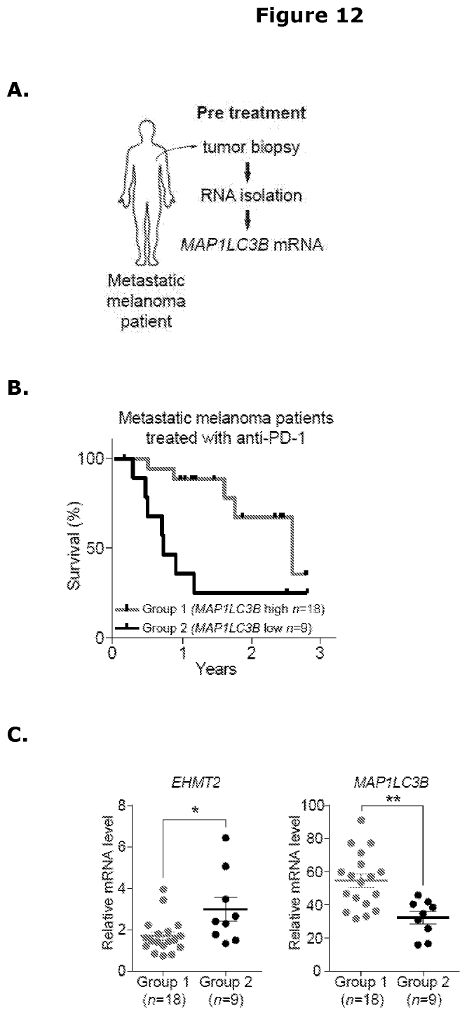

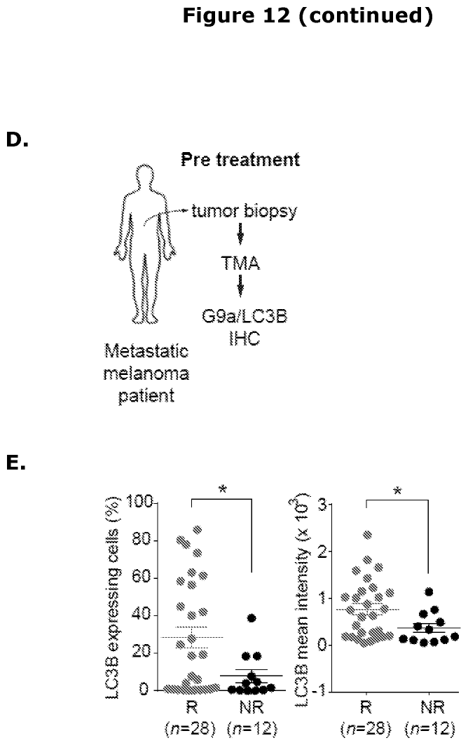

In order to determine the value of LC3B as a marker of response to immunotherapy in melanoma in-silico analysis of gene expression data (transcriptome and RNA-seq) where RNA was isolated from tumors from metastatic melanoma patients prior to anti-PD-1 therapies (pembrolizumab and nivolumab) (Hugo et al. (2016) Cell 165, 35-44) was performed (FIG. 12A). A Kaplan Meyer survival curve was generated using the expression of MAP1LC3B. Notably, patients with higher expression of MAP1LC3B had a statistically significantly longer survival (MAP1LC3B high, red) compared to patients with lower expression (MAP1LC3B low, black) (FIG. 12B; P=0.0095). There were greater than 66% of patients alive after two years in the MAP1LC3B high patient group compared to 22% in the MAP1LC3B low group. Patients that responded well to the anti-PD-1 treatment, had lower expression levels of MAP1LC3B, but higher levels of EHMT2 (G9a) expression (FIG. 12B).

To assess protein-level expression of G9a and LC3B as predictors to clinical response, IHC using specific antibodies to G9a and LC3B was performed on a tissue microarray (TMA), containing 40 melanoma samples, collected from patients prior to receiving anti-CTLA4 or anti-PD-1 treatment (FIG. 12D). This cohort was divided into “responder” (R; n=28) or “non-responder” (NR; n=12) groups. In the responding group, the level of LC3B was higher compared to non-responding group (IHC data not shown). The number and mean intensity of the LC3B-expressing cells was also investigated and showed that these were also significantly higher in the responding group (FIG. 12E).

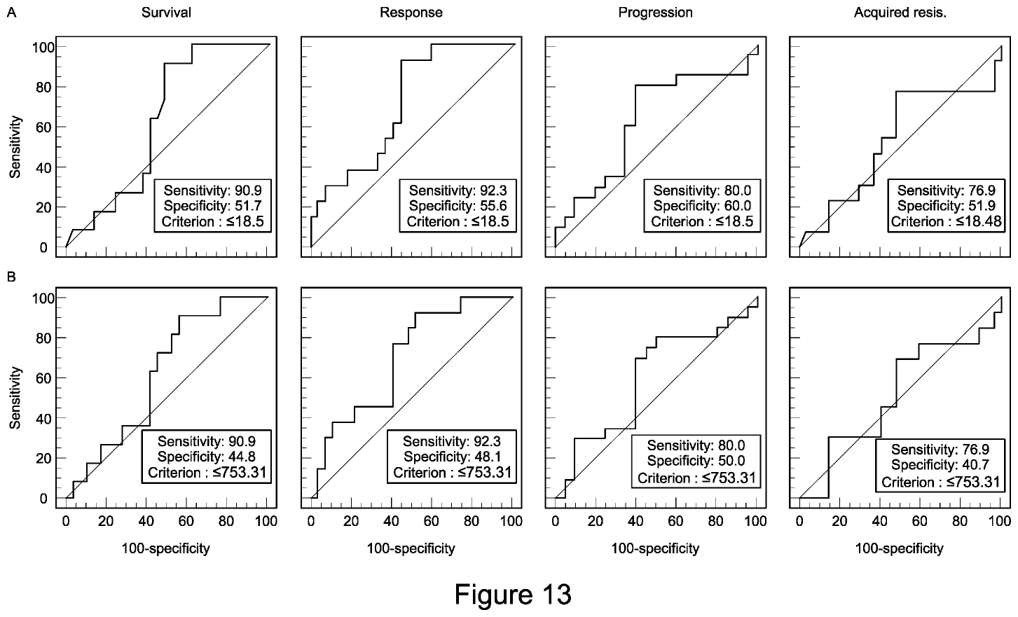

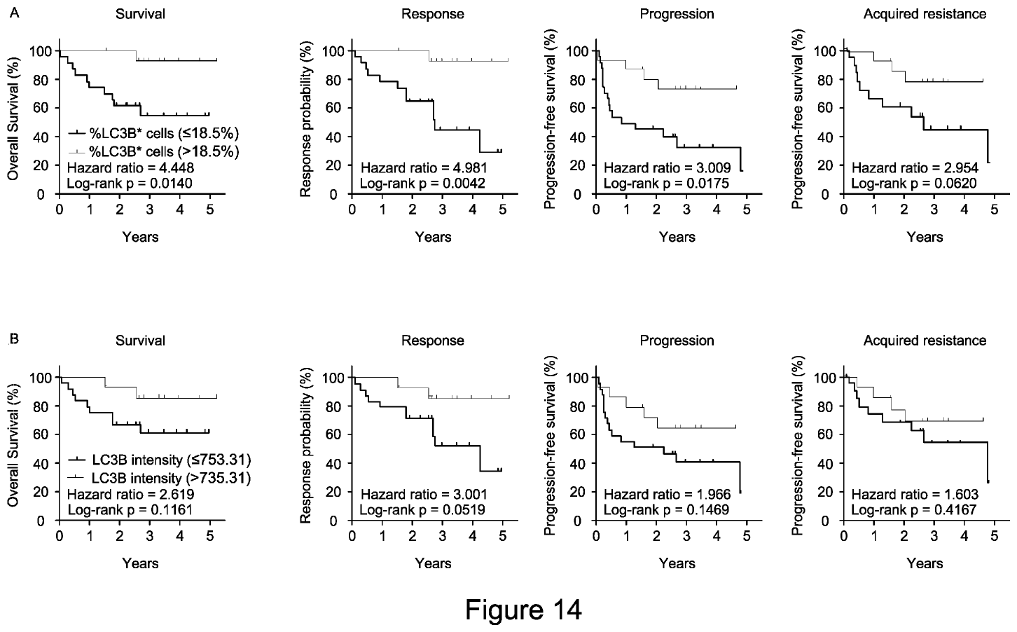

To test the association of the percentage of LC3B-positive cells (% LC3B⁺ cells) and absolute LC3B staining intensity (LC3B expression) with patient outcomes, the best cut-off using Receiver Operator Characteristic curves (ROC curves) was assessed. Based on these cut-offs (FIG. 13; percentage of LC3B-positive cells; ≤18.5; and absolute LC3B staining: ≤753.31), it was found that high percentage of LC3B⁺ cells associated significantly with better survival due to better response and lower frequency of disease progression (FIG. 14A). Higher percentage of LC3B+ cells approached significance for lower acquired resistance (PD after initial response). LC3B staining intensity showed similar trends but did not reach statistical significance for stratification of recorded endpoints (FIG. 14B). Univariate and multivariate analyses were carried out for LC3B and all other variates available in the cohort which included age (>65 vs ≤65), sex (female vs male), stage (M1c vs others), LDH (pos. vs neg.), BRAF (mut vs wt), NRAS (mut vs wt), and BRAF/NRAS mutation status (mut vs wt), where LDH pos (or LDH+) indicates that the LDH levels are increased compared to “normal” levels, and LDH neg (or LDH−) indicates that the LDH levels are in the “normal” range; and where BRAF/NRAS wt means that there are no detected mutations associated with a change in gene product function and BRAF/NRAS mut means that there is at least one detected mutation associated with a change in gene product function. Only the percentage of LC3B+ cells was significantly associated with overall survival in univariate analysis and it approached significance (P=0.056) in multivariate analysis (Table 2). For initial response to immunotherapy, both the percentage of LC3B⁺ cells and mutation status were significant in univariate analysis. In multivariate analysis only the percentage of LC3B⁺ cells and LC3B intensity associated significantly with initial response indicating them as independent prognostic factors (Table 2B). Similarly, the percentage of LC3B⁺ cells and LC3B intensity were independent prognostic factors based on multivariate analyses for disease progression (Table 2C) and acquired resistance (Table 2D).

TABLE 2
Univariate and multivariate survival analysis of LC3B expression in
immunotherapy-treated metastatic melanoma.

| | Univariate HR (95% CI) | P | Multivariate P |
|---|---|---|---|
| A. Survival | | | |
| % LC3B+ cells (>18.5% vs ≤18.5%) | 0.1166 (0.0151-0.9040) | 0.0407 | 0.056 |
| LC3B intensity (>753.31 vs ≤753.31) | 0.3125 (0.0680-1.4365) | 0.137 | 0.1172 |
| Age (>65 vs ≤65) | 0.7421 (0.2277-2.4191) | 0.6226 | 0.7267 |
| Sex (female vs male) | 0.7090 (0.1892-2.6574) | 0.6118 | 0.9527 |
| Stage (M1c vs others) | 1.5448 (0.3220-7.4107) | 0.5886 | 0.5599 |
| LDH (pos. vs neg.) | 2.7019 (0.7667-9.5218) | 0.1239 | 0.9418 |
| BRAF (mut vs WT) | 2.1047 (0.6123-7.2346) | 0.2399 | 0.9986 |
| NRAS (mut vs WT) | 0.7764 (0.1661-3.6298) | 0.749 | 0.9791 |
| Mutation Status (mut vs WT) | 2.0688 (0.5380-7.9557) | 0.2926 | 0.9969 |
| B. Response | | | |
| % LC3B+ cells (>18.5% vs ≤18.5%) | 0.0925 (0.0121-0.7071) | 0.0225 | 0.0214 |
| LC3B intensity (>753.31 vs ≤753.31) | 0.2519 (0.0562-1.1293) | 0.0731 | 0.0185 |
| Age (>65 vs ≤65) | 0.4188 (0.1294-1.3555) | 0.1485 | 0.3666 |
| Sex (female vs male) | 0.5335 (0.1456-1.9552) | 0.3455 | 0.1226 |
| Stage (M1c vs others) | 1.0033 (0.2655-3.7915) | 0.9962 | 0.7918 |
| LDH (pos. vs neg.) | 2.0110 (0.6088-6.6432) | 0.2543 | 0.0798 |
| BRAF (mut vs WT) | 3.0843 (0.9774-9.7327) | 0.056 | 0.9664 |
| NRAS (mut vs WT) | 1.0501 (0.2856-3.8606) | 0.9416 | 0.9518 |
| Mutation Status (mut vs WT) | 4.9531 (1.0879-22.551) | 0.0396 | 0.9586 |
| C. Progression | | | |
| % LC3B+ cells (>18.5% vs ≤18.5%) | 0.2836 (0.0941-0.8547) | 0.0259 | 0.0174 |
| LC3B intensity (>753.31 vs ≤753.31) | 0.4773 (0.1724-1.3213) | 0.1567 | 0.0399 |
| Age (>65 vs ≤65) | 0.6682 (0.2718-1.6428) | 0.3822 | 0.2375 |
| Sex (female vs male) | 1.2676 (0.5164-3.1117) | 0.6066 | 0.5252 |
| Stage (M1c vs others) | 1.7176 (0.4875-6.0520) | 0.4023 | 0.2569 |
| LDH (pos. vs neg.) | 2.7610 (0.9997-7.6252) | 0.0512 | 0.0135 |
| BRAF (mut VS WT) | 2.5801 (1.0408-6.3960) | 0.0418 | 0.1852 |
| NRAS (mut VS WT) | 1.3720 (0.4840-3.8893) | 0.5539 | 0.0162 |
| Mutation Status (mut vs WT) | 3.7468 (1.2361-11.357) | 0.0202 | 0.0281 |
| D. Acquired Resistance | | | |
| % LC3B+ cells (>18.5% vs ≤18.5%) | 0.3073 (0.0834-1.1319) | 0.0776 | 0.0203 |
| LC3B intensity (>753.31 vs ≤753.31) | 0.6108 (0.1848-2.0187) | 0.4213 | 0.0388 |
| Age (>65 vs ≤65) | 0.9717 (0.3087-3.0585) | 0.9611 | 0.9748 |
| Sex (female vs male) | 1.6237 (0.5403-4.8790) | 0.3903 | 0.2932 |
| Stage (M1c vs others) | 1.5659 (0.3276-7.4836) | 0.5761 | 0.8805 |
| LDH (pos. vs neg.) | 1.2534 (0.2486-6.3191) | 0.7854 | 0.2203 |
| BRAF (mut vs WT) | 2.0613 (0.6837-6.2148) | 0.2012 | 0.9597 |
| NRAS (mut vs WT) | 0.7776 (0.1634-3.7000) | 0.7532 | 0.1018 |
| Mutation Status (mut vs WT) | 2.0392 (0.5986-6.9466) | 0.2569 | 0.9662 |

The utility of G9a and LC3B as markers of response to checkpoint inhibitor therapy was then assessed using less invasive liquid biopsy (i.e. peripheral blood) samples from stage IV metastatic melanoma patients. To this end, circulating tumor cells (CTCs) from patient blood samples were isolated and the level of G9a and LC3B was examined using a diagnostically-relevant IHC staining method. The levels of G9a and LC3B were assessed in CTCs isolated from stage IV metastatic melanoma patients treated with anti-PD-1 therapy (Nivolumab). CTC collections in all cases were from metastatic melanoma patients who had already commenced anti-CTLA4 (two cycles of monotherapy; Ipilimumab) followed by anti-PD-1 therapy (one cycle; Nivolumab) for at least 3 months.

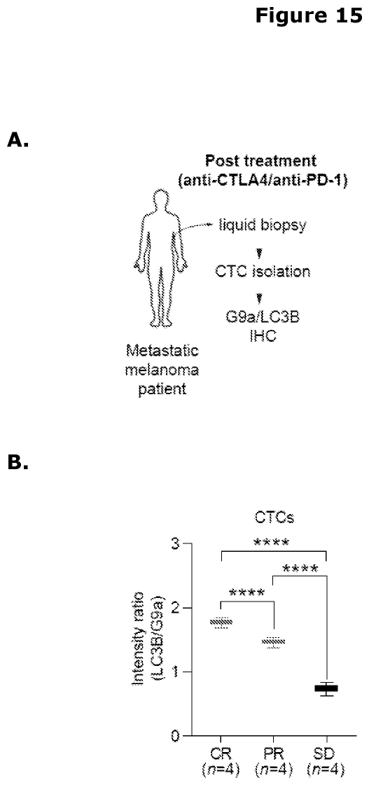

The cohort of melanoma patients included three groups: 1) complete response (CR); 2) partial response (PR) and 3) stable disease (SD) as per RECIST 1.1 (n=12 patient samples with 4 patients per group). Immunofluorescence microscopy was performed on these cells for the expression of ABCB5 to confirm their melanoma-derived CTC status. ABCB5 protein was expressed by CTCs isolated from all patient samples (data not shown). Analysis of G9a and LC3B protein expression on these CTCs showed a higher ratio of LC3B to G9a was statistically significantly associated with CR to anti-PD-1 therapy (P<0.0001; FIG. 15B). Conversely, the lowest ratio of LC3B to G9a statistically significantly associated with SD (P<0.0001; FIG. 15B). Consistently, the intermediate ratio of LC3B to G9a was associated with partial response (PR) to anti-PD-1 therapy (FIG. 15B), suggesting that G9a and LC3B expression may be used as a response marker of checkpoint inhibitor therapy in metastatic melanoma patients. Together, the analyses suggest that G9a and LC3B protein and transcript levels could be used as potential predictive and response marker to checkpoint inhibitor therapy for melanoma patients.

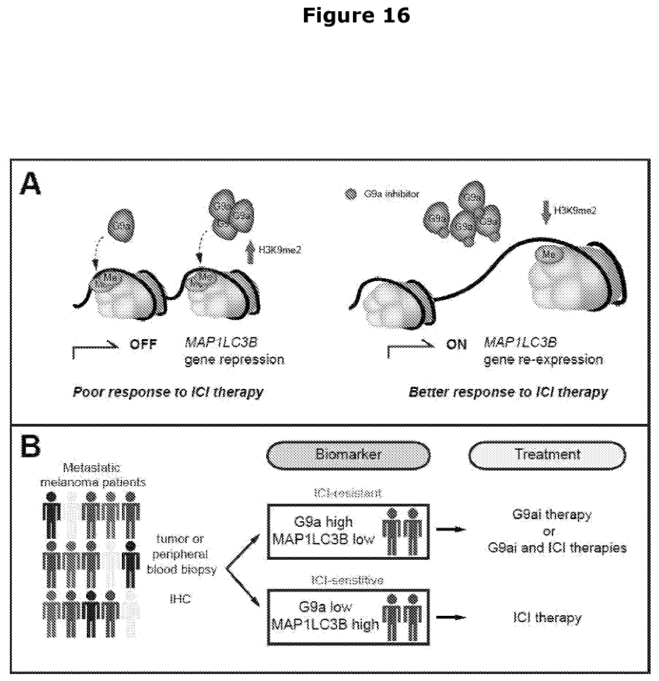

In summary, G9a and LC3B have been identified as prognostic markers in which a patient group with lower G9a and higher LC3B protein levels responded better to checkpoint inhibitor therapy. Patients with lower G9a expression and high levels of MAP1LC3B transcript associated with better survival. Perhaps more importantly, the fact that MAP1LC3B gene expression in metastatic melanoma patients prior to anti-PD-1 therapy predicted survival not only emphasizes the value of MAP1LC3B gene expression as a predictive marker of anti-PD-1 response, but also the therapeutic potential of modulating MAP1LC3B expression by targeting G9a. In addition, G9a inhibitors can be used as an adjuvant to checkpoint inhibitor therapy either potentiating the efficacy or extending the proportion of patients responding to this treatment. This is effected, at least in part, by reducing or eliminating repression of MAP1LC3B expression by G9a. G9a inhibitors can reduce histone H3K9 methylation, thereby initiating re-expression of MAP1LC3B and increasing autophagy and better response to checkpoint inhibitor blockade (FIG. 16).

### Materials and Methods

Tissue Microarray Analysis

A tissue microarray (TMA) containing melanoma tumor biopsies from 49 melanoma patients, collected prior to anti-PD-1 based immunotherapy (either pembrolizumab or nivolumab, with or without ipilimumab) and categorised as responders or non-responders, as previously described (Gide et al. Cancer Cell, 2019. 35(2): p. 238-255.e6), was stained with antibodies against G9a (abcam #ab40542 1:8000 dilution) and LC3B (Cell signaling #3868 1:14000 dilution). All multiplex tyramide labeling was performed using the Perkin Elmer Opal seven color tyramide kit (PerkinElmer #NEL797B1001KT) using the cyclic staining method. Briefly, the slide containing the TMA was dewaxed in xylene and rehydrated in water. Endogenous peroxidase was quenched and antigen retrieval was done in the microwave. Nonspecific antibody binding was blocked prior to application of the primary antibody. HRP conjugated secondary antibodies were used for primary antibody detection and signals developed with Opal tyramide. The TMA was then microwave treated to strip the primary/secondary antibody complex before repeating the staining cycle for the remaining antibodies in the panel. After the last antibody in the panel was microwave treated, the TMA was counterstained with DAPI and mounted with Dako Fluorescence Mounting Medium (Dako #S3023). Upon completion of the staining, the TMA slide was scanned using the Vectra 3.0 spectral imaging system (PerkinElmer) using the fluorescence protocol at 10 nm A from 420 nm to 720 nm, to extract fluorescent intensity information. Cell segmentation was also carried out using the InForm 4.2.1 image analysis software (PerkinElmer) and was further analysed using the FCS express 6 software (De Novo software) to determine the number and intensity of expressing cells in each individual patient sample. Receiver Operator Characteristic (ROC) curves were constructed using MedCalc® (version 12.7) for all the endpoints and the cut-off criterion for sensitivity/specificity using the DeLong et al. method (DeLong et al. Biometrics, 1988. 44(3): p. 837-45). Nine of the samples were excluded due to insufficient follow up data.

Circulating Tumor Cell Isolation and Imaging

Circulating tumor cells (CTCs) were isolated as reported previously (Boulding et al., Sci Rep, 2018. 8(1): p. 73) from Metastatic Melanoma liquid Biopsies (ethics number ETH.5.16.073) employing the RosetteSep™ Human CD45 depletion kit (Stemcell Technologies #15162) to remove CD45⁺ Cells, using density gradient centrifugation with SepMate™-50 (IVD) density gradient tubes (Stemcell Technologies #85450) and Lymphoprep™ density gradient medium (Stemcell Technologies #07861). To examine the dynamics of G9a and LC3B with chemoresistant, stem-like marker for CTCs ABCB5, CTCs were permeabilised by incubating with 1% Triton X-100 for 20 min and were probed with rabbit anti LC3B, mouse anti G9a and goat anti ABCB5 and visualized with a donkey anti-rabbit Alexa Fluor 488 (Life Technologies #A21206), anti-mouse 568 (Life Technologies #A10042) and anti-goat 633 (Life Technologies #A21082). Cover slips were mounted on glass microscope slides with ProLong Diamond Anti-fade reagent (Life Technologies #P36965). Protein targets were localised by confocal laser scanning microscopy. Single 0.5 μm sections were obtained using a Leica DMI8 microscope using 100 × oil immersion lens running LAX software. The final image was obtained by averaging four sequential images of the same section. The final image was obtained by averaging four sequential images of the same section. Digital images were analysed using ImageJ software to determine either the Total Nuclear Fluorescent Intensity (TNFI), the Total Cytoplasmic Fluorescent Intensity (TCFI) or total Fluorescent Intensity (TFI).

### Example 7

### Other Combinations of Therapy Response Markers for Melanoma

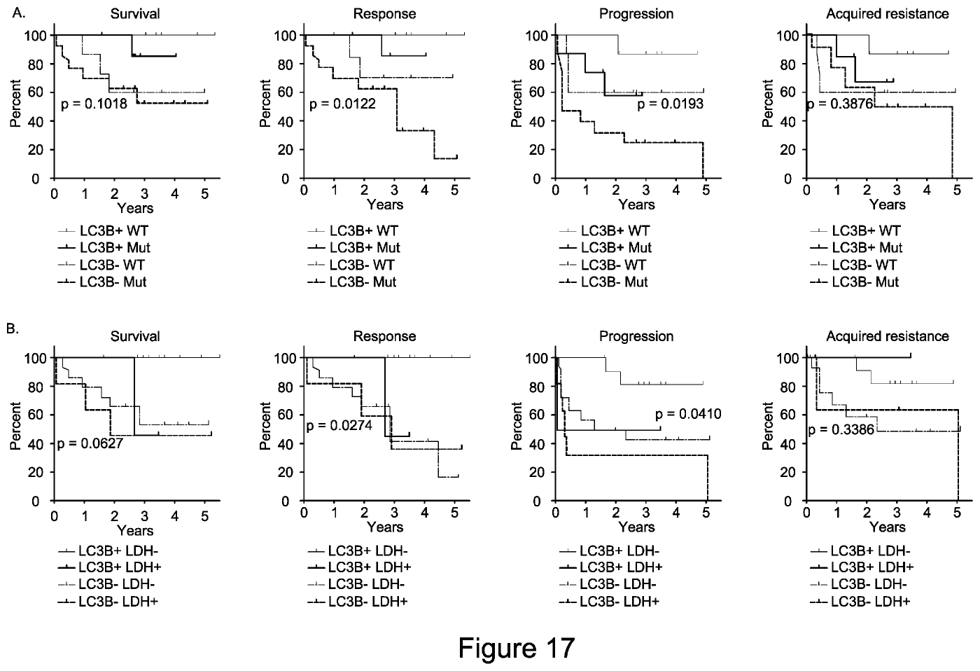

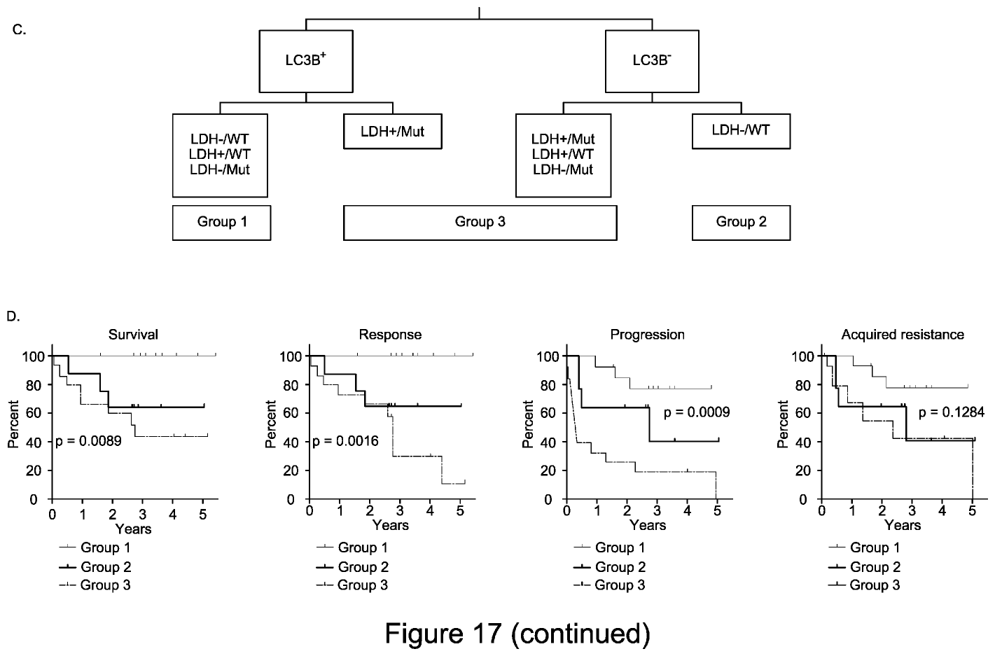

Further analysis of the prognostic value of other specific combinations of markers was performed using the same dataset described in Example 6, including the combination of percentage LC3B⁺ cells (i.e. where “LC3B⁺” means a % of LC3B-positive cells>18.5%, and LCB⁻ means a % of LC3B-positive cells of ≤18.5%) and BRAF/NRAS mutation status (Mut vs WT) (FIG. 17A) or a combination of percentage LC3B⁺ cell and LDH (FIG. 17B). Based on this analysis, patients could be classified into 3 groups: Group 1: LC3B⁺/LDH⁻/WT; LC3B⁺/LDH⁺/WT or LC3B⁺/LDH⁻/Mut; Group 2: LC3B⁻/LDH⁻/WT; and Group 3: LC3B⁺/LDH⁺/WT; LC3B⁻/LDH⁺/Mut; LC3B⁻/LDH⁺/WT; and LC3B⁻/LDH⁻/Mut (FIG. 17C). Those patients in group 3 are likely to respond only partially to checkpoint inhibitor therapy and the patients in group 3 are unlikely to respond to checkpoint inhibitor therapy (FIG. 17D). These patients are LCB⁻ (i.e. have a low percentage of LC3B⁺ cells) or are LC3B⁺ LDH⁺/Mut. It is proposed that such patients could be administered a therapeutic that increases LC3B expression, such as a G9a inhibitor, so as to then sensitize the patient to the checkpoint inhibitor therapy. In contrast, patients in group 1 are likely to respond to checkpoint inhibitor therapy (FIG. 17D) and thus are unlikely to need further adjunct therapy (e.g. G9a inhibitor therapy).

### Example 8

### Effect of G9A Inhibition on Immune Checkpoint Molecules

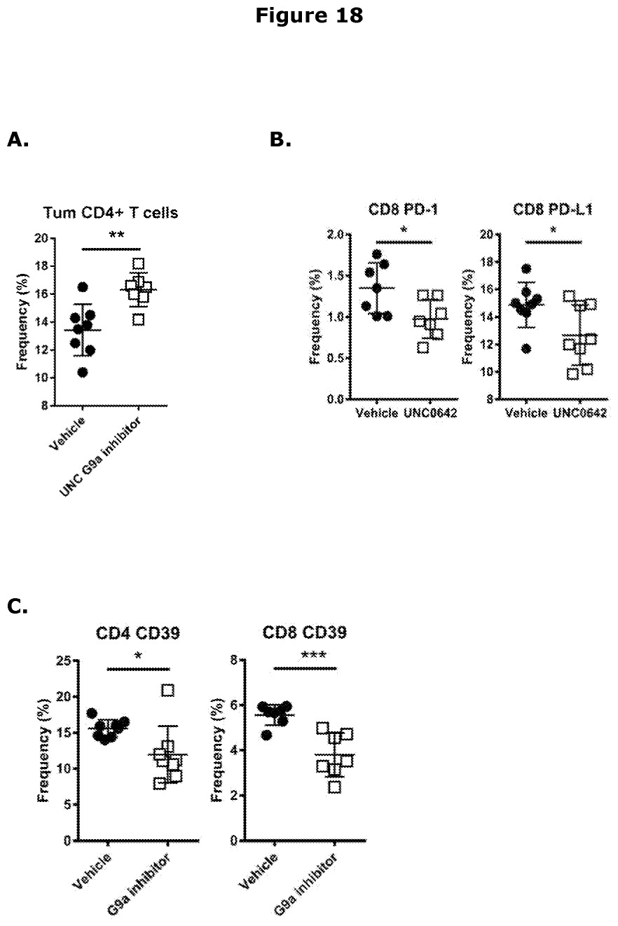

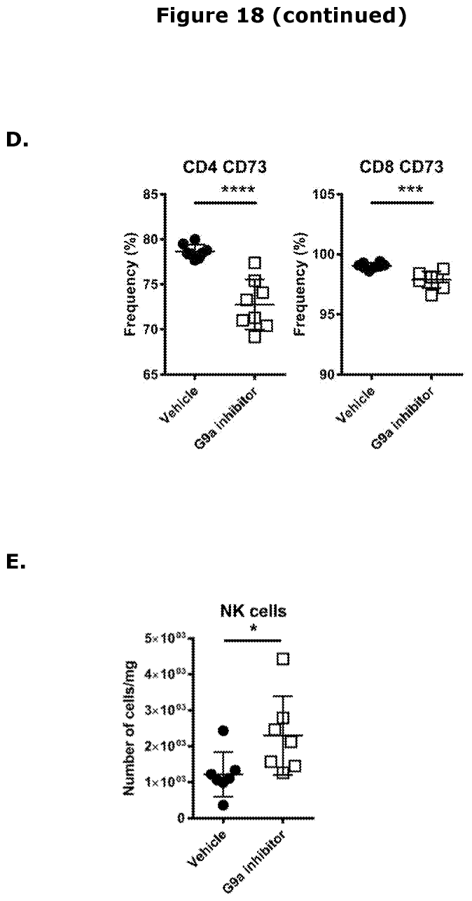

Tumor infiltrating lymphocytes (TILs) play a central role in mediating the anticancer effects of immunotherapy targeted toward immune checkpoint inhibitors. To assess the effect of G9a inhibition on TILs, TILs from AT3 tumors were exposed to a G9a inhibitor before the immune status of the cells were assessed. As shown in FIG. 18, G9a inhibition resulted in down-regulation of both PD-L1 and PD-1 in CD8+ T cells. This is an important finding as the intratumour CD8+ T cell PD-1 expression dictates response to antiPD-1 therapy (Ngiow S F, et al. (2015) Cancer research 75(18):3800-3811). These results suggest targeting G9a (EHMT2) may modulate immune checkpoint inhibitor molecules to further enhance the efficacy of immunotherapy.

### Materials and Methods

Mice

C57BL/6 mice were purchased from the ARC Animal Resources Centre and used between the ages of 6 and 16 weeks. Groups of 3 to 8 mice per experiment were used for experimental assays, to ensure adequate power to detect biological differences. All experiments were approved by the QIMR Berghofer Medical Research Institute Animal Ethics Committee.

Tumor Cell Lines

The C57BL/6 AT3 mammary adenocarcinoma (obtained from Dr. Trina Stewart in 2009, Peter MacCallum Cancer Centre, Melbourne, Australia) were maintained as previously described (Casciello et al PNAS 2017). The AT3 cell line was tested negative for mycoplasma. For in vivo experiments, 1×10⁵ cells were subcutaneously injected into mice in a 100 μL volume.

Antibodies and Reagents

Purified anti-mouse CD40 mAb (FGK4.5; 100 mg, unless indicated otherwise), PD1 mAb (RMP1-14; 250 mg), CD73 (TY/23; 250 mg) and control Ig (2A3; 250 mg; cIg) were purchased from BioXCell (West Lebanon). All antibodies and reagents were used at the dose as indicated (intraperitoneally), and tumor tissues were harvested 48 to 72 hours (unless otherwise indicated) after therapy for flow cytometry analysis.

In Vivo Treatments

AT3 tumor growth was measured using a digital caliper, and tumor volumes were presented as mean±SEM. For flow cytometry analyses of intratumor immune cells, mice bearing established AT3 tumor (days 14-19) were treated with the indicated antibodies or reagents and immune cells were isolated 48 to 72 hours post treatment.

Flow Cytometry Analysis

Tumors tissues were harvested from mice that had been treated with mAb or otherwise and processed for flow cytometry analysis. For surface staining, tumor-filtrating leukocytes (TIL) were stained with eFluor780 anti-CD45.2 (104; eBioscience), eFluor450 or Brilliant Violet 605 anti-CD4 (RM4-5; eBioscience and Biolegend), PE-Cy7 or Brilliant Violet 421 anti-CD8a (53-6.7; eBioscience and Biolegend), FITC or PE anti-TCRb (H57-597; eBioscience), PE-Cy7-anti-CD1lb (M1/70; eBioscience), eFluor450-anti-Gr1 (RB6-8C5; eBioscience), FITC- or PE-anti-PD1 (J43; eBioscience and BD Pharmingen), APC-anti-PDL1 (10F.9G2; Biolegend), PE-anti-CD27 (LG.3A10; BD Pharmingen), PE-anti-CD86 (GL1; BD Pharmingen), APCanti-CD80 (16-10A1; eBioscience), PE-anti-CD70 (FR70; BD Pharmingen), FITC-anti-CD40 (HM40-3; BD Pharmingen), Biotin-conjugated-anti-CD28 (37.51; Biolegend), PE-Cy7- or PC conjugated streptavidin (eBioscience), Alexa Fluor 488-anti-CD25 (PC61.5; eBioscience), Brilliant Violet 605-anti-CD127 (A7R34; Biolegend), PE-Cy7-anti-CD278 (ICOS; 7E.17G9; eBioscience), APC-anti-CD223 (Lag3; C9B7W; Biolegend), PEanti-CD366 (Tim3; RMT3-23; Biolegend), APC-anti-TIGIT (1G9; Biolegend), PE-Cy7 anti-CD39 (24DMS1; eBioscience), PE-anti-CD73 (TY/23; BD Pharmingen), APC-anti-CD44 (IM7; Biolegend), FITC-anti-CD62L (MEL-14; Biolegend), and respective isotype antibodies in the presence of anti-CD16/32 (2.4G2). 7AAD (Biolegend) or Zombie Aqua Fixable Viability Kit Biolegend) was used to exclude dead cells. For intracellular transcription factor staining, surface-stained cells were fixed and permeabilized using the Foxp3/Transcription Factor Staining Buffer Set (eBioscience), according to the manufacturer's protocol, and stained using eFluor450-anti-Foxp3 (FJK-16s, eBioscience), FITC-anti-Tbet (4B10; Biolegend), APC-anti-CTLA4 (UC10-4B9, eBioscience), Alexa Fluor 647-anti-Ki67 (B56), eFluor660-anti-Eomes (Dan11mag; eBioscience), and respective isotype antibodies. For intracellular staining of IFNg/TNF or IL12p40, cells were stimulated in vitro with 50 ng/mL PMA (Sigma Aldrich) and 1 mg/mL ionomycin (Sigma Aldrich), or 100 ng/mL LPS, respectively in the presence of GolgiPlug (BD Biosciences) for 4 hours, and then surface stained as aforementioned. Surface-stained cells were then fixed and permeabilized using BD Cytofix/Cytoperm (BD Biosciences) according to the manufacturer's protocol, and stained with PE-anti-IL12p40 (C15.6; BD Pharmingen), PE-anti-IFNg (XMG1.2; Bioscience), Alexa Fluor 647-anti-granzyme B (GB11; BD Pharmingen), and Brilliant Violet 605-anti-TNF (MP6-XT22; Biolegend), and respective isotype antibodies. Cells were acquired on the BD FACSCANTO II and LSR (BD Biosciences) and analysis was carried out using FlowJo (Tree Star).

Statistical Analyses

Statistical analyses were carried out using Graph Pad Prism software. Significant differences in tumor growth were determined by an unpaired t test. Significant differences in cell subsets were determined by an unpaired t test. Values of P<0.05 were considered statistically significant.

TABLE 3
Sequences

| SEQ | | |
|---|---|---|
| ID | Sequence | |
| NO: | Description | Sequence |
| 1 | Human | ACGCTGCGTGCCGCTGCTGGGTTCCGCCACGCCCGTCATGGCGGCGGCCCCGGCCGGC |
| | MAP1LC3B | TCTGGCCCCGCCCCTCGGTGACGCGTCGCGAGTCACCTGACCAGGCTGCGGGCTGAGG |
| | (LC3B) cDNA | AGATACAAGGGAAGTGGCTATCGCCAGAGTCGGATTCGCCGCCGCAGCAGCCGCCGCC |
| | (Acc. No. | CCCGGGAGCCGCCGGGACCCTCGCGTCGTCGCCGCCGCCGCCGCCCAGATCCCTGCAC |
| | NM_022818) | CATGCCGTCGGAGAAGACCTTCAAGCAGCGCCGCACCTTCGAACAAAGAGTAGAAGATG |
| | | TCCGACTTATTCGAGAGCAGCATCCAACCAAAATCCCGGTGATAATAGAACGATACAAG |
| | | GGTGAGAAGCAGCTTCCTGTTCTGGATAAAACAAAGTTCCTTGTACCTGACCATGTCAAC |
| | | ATGAGTGAGCTCATCAAGATAATTAGAAGGCGCTTACAGCTCAATGCTAATCAGGCCTTC |
| | | TTCCTGTTGGTGAACGGACACAGCATGGTCAGCGTCTCCACACCAATCTCAGAGGTGTA |
| | | TGAGAGTGAGAAAGATGAAGATGGATTCCTGTACATGGTCTATGCCTCCCAGGAGACGT |
| | | TCGGGATGAAATTGTCAGTGTAAAACCAGAAAAAATGCAGCTCTTCTAGAATTGTTTAAA |
| | | CCCTTACCAAGGAAAAAAAAGGGATGTTACCAACTGAGATCGATCAGTTCATCCAATCAC |
| | | AGATCATGAAACAGTAGTGTTCCCACCTAGGAGTGTTAGGAAGTTGTGTTTGTGTTTCAA |
| | | GCAGAAAAACTGAGCTCCAAGTGAGCACATTCAGCTTTGGAAACTATATTATTTAATGTA |
| | | GGCTAGCTTGTTTTCAAATTTTAAAAGTTTAAAAATAAAATACTTTGCATTCTAAGTTGCC |
| | | AATAAAATAGACCTTCAAGTTATTTTAATGCTCTTTTCTCACTAATAGGAACTTGTAATTCC |
| | | AGCAGTAATTTAAAGGCTTTCAGAGAGACCCTGAGTCTTCTCTTCAGGTTCACAGAACCC |
| | | GCCGCCTTTTTGGGTAGAAGTTTTCTACTCAGCTAGAGAGATCTCCCTAAGAGGATCTTT |
| | | AGGCCTGAGTTGTGAAGCGCAACCCCCGCAAAACGCATTTGCCATCACAGTTGGCACAA |
| | | ACGCAGGGTAAACGGGCTGTGTGAGAAAACGGCCCTGACTGTAAACTGCTGAAGGTCC |
| | | CTGACTCCTAAGAGAACCACACCCAAAGTCCTCACTCTTGCAGGGGTAGACATTTCTGGT |
| | | TTGGTTTGTTCTCTAGATAGTTACACACATAAAGACACCACTCAAAAGGAAACTTGAATAA |
| | | TTTATAATTTTGATCGAGTTTCTTAAAAGACCCTGGAGAAAGAGTGGCATTTCTTCTGTTT |
| | | CAGGTTTTGTCTGAGTTCAAACTAGTGCCTGTGTTGTTACGGAAAGCAGCAGTGTACCAG |
| | | TGTCACTCTGGAGTACAGCGGGAGAAACACAAAATAGTATAACTGAAAACATTAACATTC |
| | | AGACACACTCCCTTCTGCCTTCCGGCTTAAAGCTGTGGATGATCCACGTTTTTGTTTTTTT |
| | | AATGTTAAATGTGTAACTCAGTATTACTGAAAAGGTACCCACATTTTGAATAGTAGTTATC |
| | | ACTCTTAGGTCAGACAGCCATCAGAATTCTCCCACACCAAGTGCATGTCAGTTGTGGAGA |
| | | AAACATAGCAAAAAGAGCCGTACGCTCTTTACAGATACTAATGTCAAGAGTTAAACCTCC |
| | | TCAGGTTCAACCTGTGATAAAAGACTAGTGCTTCCCAGTACTTGCATGGGGTTCACTATT |
| | | TATAGTTTTCTTGGGAGTATCACAGGAAAATCACAATTACACCACTTTAGACCCTATGTGT |
| | | AGCAGGTCACAACTTACCCTTGTGTGTTTAGATGTGTATGAAATACCTGTATACGTTAGT |
| | | GAAAGCTGTTTACTGTAACGGGGAAAACCAGATTCTTTGCATCTGGGCCCTCTACTGATT |
| | | GTTAAAGGAGTTCCTGTCACCTGCTCCCCCCACCCCCGCATGCGTCTGTCCACTTGGCTA |
| | | ACTTTTAATATGTGTATTTTTACATTATGTATATTCTTAACTGGACTGTCTCGTTTAGACTG |
| | | TATACATCATATCTGACATTATTGTAACTACCGTGTGATCAGTAAGATTCCTGTAAGAAAT |
| | | ACTGCTTTTTAAGAAAAAAAATAACATGCTGAGGGGTGACCTATATCCCATGTGAGTGGT |
| | | CACTTTATTTATAGGATCTTTAAAACATTTTTAATGAACTAAGTTGAATAAAGGCACAATT |
| | | AAAAACTGTCAAAAAAAAAAAAAAAAA |
| 2 | Human | MPSEKTFKQRRTFEQRVEDVRLIREQHPTKIPVIIERYKGEKQLPVLDKTKFLVPDHVNMSEL |
| | MAP1LC3B | IKIIRRRLQLNANQAFFLLVNGHSMVSVSTPISEVYESEKDEDGFLYMVYASQETFGMKLSV |
| | (LC3B) | |
| | polypeptide | |
| | (Acc. No. | |
| | Q9GZQ8) | |
| 3 | Human EHMT2 | AGAGATGCGGGGTCTACCGAGAGGGAGGGGGTTGATGCGGGCCCGGGGGAGGGGTCG |
| | (G9a) cDNA, | TGCGGCCCCTCCGGGCAGCCGAGGCCGCGGAAGGGGGGGGCCCCACAGAGGAAGAG |
| | transcript | GTAGGCCCCGGAGCCTACTCTCTCTTCCCAGGGCCCAGGCATCCTGGACCCCCCAACTC |
| | variant 1 (Acc. | TCTACTGGGCTGACCAGCCCTCCTGTCCCTTGTCTCCCCTCCCAGGGGGAGGCCCCCGC |
| | No. | TGAGATGGGGGCGCTGCTGCTGGAGAAGGAAACCAGAGGAGCCACCGAGAGAGTTCAT |
| | NM_001289413.1) | GGCTCTTTGGGGGACACCCCTCGTAGTGAAGAAACCCTGCCCAAGGCCACCCCCGACTC |
| | | CCTGGAGCCTGCTGGCCCCTCATCTCCAGCCTCTGTCACTGTCACTGTTGGTGATGAGG |
| | | GGGCTGACACCCCTGTAGGGGCTACACCACTCATTGGGGATGAATCTGAGAATCTTGAG |
| | | GGAGATGGGGACCTCCGTGGGGGCCGGATCCTGCTGGGCCATGCCACAAAGTCATTCC |
| | | CCTCTTCCCCCAGCAAGGGGGGTTCCTGTCCTAGCCGGGCCAAGATGTCAATGACAGGG |
| | | GCGGGAAAATCACCTCCATCTGTCCAGAGTTTGGCTATGAGGCTACTGAGTATGCCAGG |
| | | AGCCCAGGGAGCTGCAGCAGCAGGGTCTGAACCCCCTCCAGCCACCACGAGCCCAGAG |
| | | GGACAGCCCAAGGTCCACCGAGCCCGCAAAACCATGTCCAAACCAGGAAATGGACAGC |
| | | CCCCGGTCCCTGAGAAGCGGCCCCCTGAAATACAGCATTTCCGCATGAGTGATGATGTC |
| | | CACTCACTGGGAAAGGTGACCTCAGATCTGGCCAAAAGGAGGAAGCTGAACTCAGGAG |
| | | GTGGCCTGTCAGAGGAGTTAGGTTCTGCCCGGCGTTCAGGAGAAGTGACCCTGACGAA |
| | | AGGGGACCCCGGGTCCCTGGAGGAGTGGGAGACGGTGGTGGGTGATGACTTCAGTCTC |
| | | TACTATGATTCCTACTCTGTGGATGAGCGCGTGGACTCCGACAGCAAGTCTGAAGTTGA |
| | | AGCTCTAACTGAACAACTAAGTGAAGAGGAGGAGGAGGAAGAGGAGGAAGAAGAAGAA |
| | | GAGGAAGAGGAGGAGGAAGAGGAAGAAGAAGAGGAAGATGAGGAGTCAGGGAATCAG |
| | | TCAGATAGGAGTGGTTCCAGTGGCCGGCGCAAGGCCAAGAAGAAATGGCGAAAAGACA |
| | | GCCCATGGGTGAAGCCGTCTCGGAAACGGCGCAAGCGGGAGCCTCCGCGGGCCAAGG |
| | | AGCCACGAGGGGTGTCCAATGACACATCTTCGCTGGAGACAGAGCGAGGGTTTGAGGA |
| | | GTTGCCCCTGTGCAGCTGCCGCATGGAGGCACCCAAGATTGACCGCATCAGCGAGAGG |
| | | GCGGGGCACAAGTGCATGGCCACTGAGAGTGTGGACGGAGAGCTGTCAGGCTGCAATG |
| | | CCGCCATCCTCAAGCGGGAGACCATGAGGCCATCCAGCCGTGTGGCCCTGATGGTGCT |
| | | CTGTGAGACCCACCGCGCCCGCATGGTCAAACACCACTGCTGCCCGGGCTGCGGCTACT |
| | | TCTGCACGGCGGGCACCTTCCTGGAGTGCCACCCTGACTTCCGTGTGGCCCACCGCTTC |
| | | CACAAGGCCTGTGTGTCTCAGCTGAATGGGATGGTCTTCTGTCCCCACTGTGGGGAGGA |
| | | TGCTTCTGAAGCTCAAGAGGTGACCATCCCCCGGGGTGACGGGGTGACCCCACCGGCC |
| | | GGCACTGCAGCTCCTGCACCCCCACCCCTGTCCCAGGATGTCCCCGGGAGAGCAGACA |
| | | CTTCTCAGCCCAGTGCCCGGATGCGAGGGCATGGGGAACCCCGGCGCCCGCCCTGCGA |
| | | TCCCCTGGCTGACACCATTGACAGCTCAGGGCCCTCCCTGACCCTGCCCAATGGGGGCT |
| | | GCCTTTCAGCCGTGGGGCTGCCACTGGGGCCAGGCCGGGAGGCCCTGGAAAAGGCCCT |
| | | GGTCATCCAGGAGTCAGAGAGGCGGAAGAAGCTCCGTTTCCACCCTCGGCAGTTGTACC |
| | | TGTCCGTGAAGCAGGGCGAGCTGCAGAAGGTGATCCTGATGCTGTTGGACAACCTGGA |
| | | CCCCAACTTCCAGAGCGACCAGCAGAGCAAGCGCACGCCCCTGCATGCAGCCGCCCAG |
| | | AAGGGCTCCGTGGAGATCTGCCATGTGCTGCTGCAGGCTGGAGCCAACATAAATGCAGT |
| | | GGACAAACAGCAGCGGACGCCACTGATGGAGGCCGTGGTGAACAACCACCTGGAGGTA |
| | | GCCCGTTACATGGTGCAGCGTGGTGGCTGTGTCTATAGCAAGGAGGAGGACGGTTCCA |
| | | CCTGCCTCCACCACGCAGCCAAAATCGGGAACTTGGAGATGGTCAGCCTGCTGCTGAGC |
| | | ACAGGACAGGTGGACGTCAACGCCCAGGACAGTGGGGGGTGGACGCCCATCATCTGGG |
| | | CTGCAGAGCACAAGCACATCGAGGTGATCCGCATGCTACTGACGCGGGGCGCCGACGT |
| | | CACCCTCACTGACAACGAGGAGAACATCTGCCTGCACTGGGCCTCCTTCACGGGCAGCG |
| | | CCGCCATCGCCGAAGTCCTTCTGAATGCGCGCTGTGACCTCCATGCTGTCAACTACCAT |
| | | GGGGACACCCCCCTGCACATCGCAGCTCGGGAGAGCTACCATGACTGCGTGCTGTTATT |
| | | CCTGTCACGTGGGGCCAACCCTGAGCTGCGGAACAAAGAGGGGGACACAGCATGGGAC |
| | | CTGACTCCCGAGCGCTCCGACGTGTGGTTTGCGCTTCAACTCAACCGCAAGCTCCGACT |
| | | TGGGGTGGGAAATCGGGCCATCCGCACAGAGAAGATCATCTGCCGGGACGTGGCTCGG |
| | | GGCTATGAGAACGTGCCCATTCCCTGTGTCAACGGTGTGGATGGGGAGCCCTGCCCTGA |
| | | GGATTACAAGTACATCTCAGAGAACTGCGAGACGTCCACCATGAACATCGATCGCAACA |
| | | TCACCCACCTGCAGCACTGCACGTGTGTGGACGACTGCTCTAGCTCCAACTGCCTGTGC |
| | | GGCCAGCTCAGCATCCGGTGCTGGTATGACAAGGATGGGCGATTGCTCCAGGAATTTAA |
| | | CAAGATTGAGCCTCCGCTGATTTTCGAGTGTAACCAGGCGTGCTCATGCTGGAGAAACT |
| | | GCAAGAACCGGGTCGTACAGAGTGGCATCAAGGTGCGGCTACAGCTCTACCGAACAGC |
| | | CAAGATGGGCTGGGGGGTCCGCGCCCTGCAGACCATCCCACAGGGGACCTTCATCTGC |
| | | GAGTATGTCGGGGAGCTGATCTCTGATGCTGAGGCTGATGTGAGAGAGGATGATTCTTA |
| | | CCTCTTCGACTTAGACAACAAGGATGGAGAGGTGTACTGCATAGATGCCCGTTACTATG |
| | | GCAACATCAGCCGCTTCATCAACCACCTGTGTGACCCCAACATCATTCCCGTCCGGGTCT |
| | | TCATGCTGCACCAAGACCTGCGATTTCCACGCATCGCCTTCTTCAGTTCCCGAGACATCC |
| | | GGACTGGGGAGGAGCTAGGGTTTGACTATGGCGACCGCTTCTGGGACATCAAAAGCAA |
| | | ATATTTCACCTGCCAATGTGGCTCTGAGAAGTGCAAGCACTCAGCCGAAGCCATTGCCCT |
| | | GGAGCAGAGCCGTCTGGCCCGCCTGGACCCACACCCTGAGCTGCTGCCCGAGCTCGGC |
| | | TCCCTGCCCCCTGTCAACACATGAGAACGGACCACACCCTCTCTCCCCAGCATGGATGG |
| | | CCACAGCTCAGCCGCCTCCTCTGCCACCAGCTGCTCGCAGCCCATGCCTGGGGGTGCTG |
| | | CCATCTTCTCTCCCCACCACCCTTTCACACATTCCTGACCAGAGATCCCAGCCAGGCCCT |
| | | GGAGGTCTGACAGCCCCTCCCTCCCAGAGCTGGTTCCTCCCTGGGAGGGCAACTTCAGG |
| | | GCTGGCCACCCCCCGTGTTCCCCATCCTCAGTTGAAGTTTGATGAATTGAAGTCGGGCC |
| | | TCTATGCCAACTGGTTCCTTTTGTTCTCAATAAATGTTGGGTTTGGTAATAAAAAAAAAAA |
| | | AAAAAA |
| 4 | Human EHMT2 | MRGLPRGRGLMRARGRGRAAPPGSRGRGRGGPHRGRGRPRSLLSLPRAQASWTPQLSTG |
| | (G9a) | LTSPPVPCLPSQGEAPAEMGALLLEKETRGATERVHGSLGDTPRSEETLPKATPDSLEPAGPS |
| | polypeptide, | SPASVTVTVGDEGADTPVGATPLIGDESENLEGDGDLRGGRILLGHATKSFPSSPSKGGSC |
| | variant 1 (Acc. | PSRAKMSMTGAGKSPPSVQSLAMRLLSMPGAQGAAAAGSEPPPATTSPEGQPKVHRARKT |
| | No. | MSKPGNGQPPVPEKRPPEIQHFRMSDDVHSLGKVTSDLAKRRKLNSGGGLSEELGSARRS |
| | NP_001276342.1) | GEVTLTKGDPGSLEEWETVVGDDFSLYYDSYSVDERVDSDSKSEVEALTEQLSEEEEEEEE |
| | | EEEEEEEEEEEEEEEEDEESGNQSDRSGSSGRRKAKKKWRKDSPWVKPSRKRRKREPPRA |
| | | KEPRGVSNDTSSLETERGFEELPLCSCRMEAPKIDRISERAGHKCMATESVDGELSGCNAAI |
| | | LKRETMRPSSRVALMVLCETHRARMVKHHCCPGCGYFCTAGTFLECHPDFRVAHRFHKACV |
| | | SQLNGMVFCPHCGEDASEAQEVTIPRGDGVTPPAGTAAPAPPPLSQDVPGRADTSQPSAR |
| | | MRGHGEPRRPPCDPLADTIDSSGPSLTLPNGGCLSAVGLPLGPGREALEKALVIQESERRKK |
| | | LRFHPRQLYLSVKQGELQKVILMLLDNLDPNFQSDQQSKRTPLHAAAQKGSVEICHVLLQA |
| | | GANINAVDKQQRTPLMEAVVNNHLEVARYMVQRGGCVYSKEEDGSTCLHHAAKIGNLEMV |
| | | SLLLSTGQVDVNAQDSGGWTPIIWAAEHKHIEVIRMLLTRGADVTLTDNEENICLHWASFT |
| | | GSAAIAEVLLNARCDLHAVNYHGDTPLHIAARESYHDCVLLFLSRGANPELRNKEGDTAWD |
| | | LTPERSDVWFALQLNRKLRLGVGNRAIRTEKIICRDVARGYENVPIPCVNGVDGEPCPEDYK |
| | | YISENCETSTMNIDRNITHLQHCTCVDDCSSSNCLCGQLSIRCWYDKDGRLLQEFNKIEPPL |
| | | IFECNQACSCWRNCKNRVVQSGIKVRLQLYRTAKMGWGVRALQTIPQGTFICEYVGELISD |
| | | AEADVREDDSYLFDLDNKDGEVYCIDARYYGNISRFINHLCDPNIIPVRVFMLHQDLRFPRIA |
| | | FFSSRDIRTGEELGFDYGDRFWDIKSKYFTCQCGSEKCKHSAEAIALEQSRLARLDPHPELL |
| | | PELGSLPPVNT |
| 5 | Human EHMT2 | GCGCCGGCTCGGCCCCCAGCGCAAGCGGCGATGGCGGCGGCGGCGGGAGCTGCAGCG |
| | (G9a) cDNA, | GCGGCGGCCGCCGAGGGGGAGGCCCCCGCTGAGATGGGGGCGCTGCTGCTGGAGAAG |
| | transcript | GAAACCAGAGGAGCCACCGAGAGAGTTCATGGCTCTTTGGGGGACACCCCTCGTAGTG |
| | variant 2 (Acc. | AAGAAACCCTGCCCAAGGCCACCCCCGACTCCCTGGAGCCTGCTGGCCCCTCATCTCCA |
| | No. | GCCTCTGTCACTGTCACTGTTGGTGATGAGGGGGCTGACACCCCTGTAGGGGCTACACC |
| | NM_006709.4) | ACTCATTGGGGATGAATCTGAGAATCTTGAGGGAGATGGGGACCTCCGTGGGGGCCGG |
| | | ATCCTGCTGGGCCATGCCACAAAGTCATTCCCCTCTTCCCCCAGCAAGGGGGGTTCCTG |
| | | TCCTAGCCGGGCCAAGATGTCAATGACAGGGGCGGGAAAATCACCTCCATCTGTCCAGA |
| | | GTTTGGCTATGAGGCTACTGAGTATGCCAGGAGCCCAGGGAGCTGCAGCAGCAGGGTC |
| | | TGAACCCCCTCCAGCCACCACGAGCCCAGAGGGACAGCCCAAGGTCCACCGAGCCCGC |
| | | AAAACCATGTCCAAACCAGGAAATGGACAGCCCCCGGTCCCTGAGAAGCGGCCCCCTGA |
| | | AATACAGCATTTCCGCATGAGTGATGATGTCCACTCACTGGGAAAGGTGACCTCAGATCT |
| | | GGCCAAAAGGAGGAAGCTGAACTCAGGAGGTGGCCTGTCAGAGGAGTTAGGTTCTGCC |
| | | CGGCGTTCAGGAGAAGTGACCCTGACGAAAGGGGACCCCGGGTCCCTGGAGGAGTGG |
| | | GAGACGGTGGTGGGTGATGACTTCAGTCTCTACTATGATTCCTACTCTGTGGATGAGCG |
| | | CGTGGACTCCGACAGCAAGTCTGAAGTTGAAGCTCTAACTGAACAACTAAGTGAAGAGG |
| | | AGGAGGAGGAAGAGGAGGAAGAAGAAGAAGAGGAAGAGGAGGAGGAAGAGGAAGAA |
| | | GAAGAGGAAGATGAGGAGTCAGGGAATCAGTCAGATAGGAGTGGTTCCAGTGGCCGGC |
| | | GCAAGGCCAAGAAGAAATGGCGAAAAGACAGCCCATGGGTGAAGCCGTCTCGGAAACG |
| | | GCGCAAGCGGGAGCCTCCGCGGGCCAAGGAGCCACGAGGAGTGAATGGTGTGGGCTC |
| | | CTCAGGCCCCAGTGAGTACATGGAGGTCCCTCTGGGGTCCCTGGAGCTGCCCAGCGAG |
| | | GGGACCCTCTCCCCCAACCACGCTGGGGTGTCCAATGACACATCTTCGCTGGAGACAGA |
| | | GCGAGGGTTTGAGGAGTTGCCCCTGTGCAGCTGCCGCATGGAGGCACCCAAGATTGAC |
| | | CGCATCAGCGAGAGGGCGGGGCACAAGTGCATGGCCACTGAGAGTGTGGACGGAGAG |
| | | CTGTCAGGCTGCAATGCCGCCATCCTCAAGCGGGAGACCATGAGGCCATCCAGCCGTGT |
| | | GGCCCTGATGGTGCTCTGTGAGACCCACCGCGCCCGCATGGTCAAACACCACTGCTGCC |
| | | CGGGCTGCGGCTACTTCTGCACGGCGGGCACCTTCCTGGAGTGCCACCCTGACTTCCGT |
| | | GTGGCCCACCGCTTCCACAAGGCCTGTGTGTCTCAGCTGAATGGGATGGTCTTCTGTCC |
| | | CCACTGTGGGGAGGATGCTTCTGAAGCTCAAGAGGTGACCATCCCCCGGGGTGACGGG |
| | | GTGACCCCACCGGCCGGCACTGCAGCTCCTGCACCCCCACCCCTGTCCCAGGATGTCCC |
| | | CGGGAGAGCAGACACTTCTCAGCCCAGTGCCCGGATGCGAGGGCATGGGGAACCCCGG |
| | | CGCCCGCCCTGCGATCCCCTGGCTGACACCATTGACAGCTCAGGGCCCTCCCTGACCCT |
| | | GCCCAATGGGGGCTGCCTTTCAGCCGTGGGGCTGCCACTGGGGCCAGGCCGGGAGGC |
| | | CCTGGAAAAGGCCCTGGTCATCCAGGAGTCAGAGAGGCGGAAGAAGCTCCGTTTCCAC |
| | | CCTCGGCAGTTGTACCTGTCCGTGAAGCAGGGCGAGCTGCAGAAGGTGATCCTGATGCT |
| | | GTTGGACAACCTGGACCCCAACTTCCAGAGCGACCAGCAGAGCAAGCGCACGCCCCTG |
| | | CATGCAGCCGCCCAGAAGGGCTCCGTGGAGATCTGCCATGTGCTGCTGCAGGCTGGAG |
| | | CCAACATAAATGCAGTGGACAAACAGCAGCGGACGCCACTGATGGAGGCCGTGGTGAA |
| | | CAACCACCTGGAGGTAGCCCGTTACATGGTGCAGCGTGGTGGCTGTGTCTATAGCAAGG |
| | | AGGAGGACGGTTCCACCTGCCTCCACCACGCAGCCAAAATCGGGAACTTGGAGATGGTC |
| | | AGCCTGCTGCTGAGCACAGGACAGGTGGACGTCAACGCCCAGGACAGTGGGGGGTGG |
| | | ACGCCCATCATCTGGGCTGCAGAGCACAAGCACATCGAGGTGATCCGCATGCTACTGAC |
| | | GCGGGGCGCCGACGTCACCCTCACTGACAACGAGGAGAACATCTGCCTGCACTGGGCC |
| | | TCCTTCACGGGCAGCGCCGCCATCGCCGAAGTCCTTCTGAATGCGCGCTGTGACCTCCA |
| | | TGCTGTCAACTACCATGGGGACACCCCCCTGCACATCGCAGCTCGGGAGAGCTACCATG |
| | | ACTGCGTGCTGTTATTCCTGTCACGTGGGGCCAACCCTGAGCTGCGGAACAAAGAGGG |
| | | GGACACAGCATGGGACCTGACTCCCGAGCGCTCCGACGTGTGGTTTGCGCTTCAACTCA |
| | | ACCGCAAGCTCCGACTTGGGGTGGGAAATCGGGCCATCCGCACAGAGAAGATCATCTG |
| | | CCGGGACGTGGCTCGGGGCTATGAGAACGTGCCCATTCCCTGTGTCAACGGTGTGGAT |
| | | GGGGAGCCCTGCCCTGAGGATTACAAGTACATCTCAGAGAACTGCGAGACGTCCACCAT |
| | | GAACATCGATCGCAACATCACCCACCTGCAGCACTGCACGTGTGTGGACGACTGCTCTA |
| | | GCTCCAACTGCCTGTGCGGCCAGCTCAGCATCCGGTGCTGGTATGACAAGGATGGGCG |
| | | ATTGCTCCAGGAATTTAACAAGATTGAGCCTCCGCTGATTTTCGAGTGTAACCAGGCGTG |
| | | CTCATGCTGGAGAAACTGCAAGAACCGGGTCGTACAGAGTGGCATCAAGGTGCGGCTA |
| | | CAGCTCTACCGAACAGCCAAGATGGGCTGGGGGGTCCGCGCCCTGCAGACCATCCCAC |
| | | AGGGGACCTTCATCTGCGAGTATGTCGGGGAGCTGATCTCTGATGCTGAGGCTGATGTG |
| | | AGAGAGGATGATTCTTACCTCTTCGACTTAGACAACAAGGATGGAGAGGTGTACTGCAT |
| | | AGATGCCCGTTACTATGGCAACATCAGCCGCTTCATCAACCACCTGTGTGACCCCAACAT |
| | | CATTCCCGTCCGGGTCTTCATGCTGCACCAAGACCTGCGATTTCCACGCATCGCCTTCTT |
| | | CAGTTCCCGAGACATCCGGACTGGGGAGGAGCTAGGGTTTGACTATGGCGACCGCTTCT |
| | | GGGACATCAAAAGCAAATATTTCACCTGCCAATGTGGCTCTGAGAAGTGCAAGCACTCA |
| | | GCCGAAGCCATTGCCCTGGAGCAGAGCCGTCTGGCCCGCCTGGACCCACACCCTGAGC |
| | | TGCTGCCCGAGCTCGGCTCCCTGCCCCCTGTCAACACATGAGAACGGACCACACCCTCT |
| | | CTCCCCAGCATGGATGGCCACAGCTCAGCCGCCTCCTCTGCCACCAGCTGCTCGCAGCC |
| | | CATGCCTGGGGGTGCTGCCATCTTCTCTCCCCACCACCCTTTCACACATTCCTGACCAGA |
| | | GATCCCAGCCAGGCCCTGGAGGTCTGACAGCCCCTCCCTCCCAGAGCTGGTTCCTCCCT |
| | | GGGAGGGCAACTTCAGGGCTGGCCACCCCCCGTGTTCCCCATCCTCAGTTGAAGTTTGA |
| | | TGAATTGAAGTCGGGCCTCTATGCCAACTGGTTCCTTTTGTTCTCAATAAATGTTGGGTTT |
| | | GGTAATAAAAAAAAAAAAAAAAA |
| 6 | Human EHMT2 | MAAAAGAAAAAAAEGEAPAEMGALLLEKETRGATERVHGSLGDTPRSEETLPKATPDSLEP |
| | (G9a) | AGPSSPASVTVTVGDEGADTPVGATPLIGDESENLEGDGDLRGGRILLGHATKSFPSSPSK |
| | polypeptide, | GGSCPSRAKMSMTGAGKSPPSVQSLAMRLLSMPGAQGAAAAGSEPPPATTSPEGQPKVHR |
| | variant 2 (Acc. | ARKTMSKPGNGQPPVPEKRPPEIQHFRMSDDVHSLGKVTSDLAKRRKLNSGGGLSEELGS |
| | No. | ARRSGEVTLTKGDPGSLEEWETVVGDDFSLYYDSYSVDERVDSDSKSEVEALTEQLSEEEE |
| | NP_006700.3) | EEEEEEEEEEEEEEEEEEEEDEESGNQSDRSGSSGRRKAKKKWRKDSPWVKPSRKRRKRE |
| | | PPRAKEPRGVNGVGSSGPSEYMEVPLGSLELPSEGTLSPNHAGVSNDTSSLETERGFEELPL |
| | | CSCRMEAPKIDRISERAGHKCMATESVDGELSGCNAAILKRETMRPSSRVALMVLCETHRA |
| | | RMVKHHCCPGCGYFCTAGTFLECHPDFRVAHRFHKACVSQLNGMVFCPHCGEDASEAQEV |
| | | TIPRGDGVTPPAGTAAPAPPPLSQDVPGRADTSQPSARMRGHGEPRRPPCDPLADTIDSSG |
| | | PSLTLPNGGCLSAVGLPLGPGREALEKALVIQESERRKKLRFHPRQLYLSVKQGELQKVILML |
| | | LDNLDPNFQSDQQSKRTPLHAAAQKGSVEICHVLLQAGANINAVDKQQRTPLMEAVVNNH |
| | | LEVARYMVQRGGCVYSKEEDGSTCLHHAAKIGNLEMVSLLLSTGQVDVNAQDSGGWTPII |
| | | WAAEHKHIEVIRMLLTRGADVTLTDNEENICLHWASFTGSAAIAEVLLNARCDLHAVNYHG |
| | | DTPLHIAARESYHDCVLLFLSRGANPELRNKEGDTAWDLTPERSDVWFALQLNRKLRLGVG |
| | | NRAIRTEKIICRDVARGYENVPIPCVNGVDGEPCPEDYKYISENCETSTMNIDRNITHLQHCT |
| | | CVDDCSSSNCLCGQLSIRCWYDKDGRLLQEFNKIEPPLIFECNQACSCWRNCKNRVVQSG |
| | | IKVRLQLYRTAKMGWGVRALQTIPQGTFICEYVGELISDAEADVREDDSYLFDLDNKDGEVY |
| | | CIDARYYGNISRFINHLCDPNIIPVRVFMLHQDLRFPRIAFFSSRDIRTGEELGFDYGDRFWD |
| | | IKSKYFTCQCGSEKCKHSAEAIALEQSRLARLDPHPELLPELGSLPPVNT |
| 7 | Human EHMT2 | GCGCCGGCTCGGCCCCCAGCGCAAGCGGCGATGGCGGCGGCGGCGGGAGCTGCAGCG |
| | (G9a) cDNA, | GCGGCGGCCGCCGAGGGGGAGGCCCCCGCTGAGATGGGGGCGCTGCTGCTGGAGAAG |
| | transcript | GAAACCAGAGGAGCCACCGAGAGAGTTCATGGCTCTTTGGGGGACACCCCTCGTAGTG |
| | variant 3 (Acc. | AAGAAACCCTGCCCAAGGCCACCCCCGACTCCCTGGAGCCTGCTGGCCCCTCATCTCCA |
| | No. | GCCTCTGTCACTGTCACTGTTGGTGATGAGGGGGCTGACACCCCTGTAGGGGCTACACC |
| | NM_025256.6) | ACTCATTGGGGATGAATCTGAGAATCTTGAGGGAGATGGGGACCTCCGTGGGGGCCGG |
| | | ATCCTGCTGGGCCATGCCACAAAGTCATTCCCCTCTTCCCCCAGCAAGGGGGGTTCCTG |
| | | TCCTAGCCGGGCCAAGATGTCAATGACAGGGGCGGGAAAATCACCTCCATCTGTCCAGA |
| | | GTTTGGCTATGAGGCTACTGAGTATGCCAGGAGCCCAGGGAGCTGCAGCAGCAGGGTC |
| | | TGAACCCCCTCCAGCCACCACGAGCCCAGAGGGACAGCCCAAGGTCCACCGAGCCCGC |
| | | AAAACCATGTCCAAACCAGGAAATGGACAGCCCCCGGTCCCTGAGAAGCGGCCCCCTGA |
| | | AATACAGCATTTCCGCATGAGTGATGATGTCCACTCACTGGGAAAGGTGACCTCAGATCT |
| | | GGCCAAAAGGAGGAAGCTGAACTCAGGAGGTGGCCTGTCAGAGGAGTTAGGTTCTGCC |
| | | CGGCGTTCAGGAGAAGTGACCCTGACGAAAGGGGACCCCGGGTCCCTGGAGGAGTGG |
| | | GAGACGGTGGTGGGTGATGACTTCAGTCTCTACTATGATTCCTACTCTGTGGATGAGCG |
| | | CGTGGACTCCGACAGCAAGTCTGAAGTTGAAGCTCTAACTGAACAACTAAGTGAAGAGG |
| | | AGGAGGAGGAAGAGGAGGAAGAAGAAGAAGAGGAAGAGGAGGAGGAAGAGGAAGAA |
| | | GAAGAGGAAGATGAGGAGTCAGGGAATCAGTCAGATAGGAGTGGTTCCAGTGGCCGGC |
| | | GCAAGGCCAAGAAGAAATGGCGAAAAGACAGCCCATGGGTGAAGCCGTCTCGGAAACG |
| | | GCGCAAGCGGGAGCCTCCGCGGGCCAAGGAGCCACGAGGGGTGTCCAATGACACATCT |
| | | TCGCTGGAGACAGAGCGAGGGTTTGAGGAGTTGCCCCTGTGCAGCTGCCGCATGGAGG |
| | | CACCCAAGATTGACCGCATCAGCGAGAGGGCGGGGCACAAGTGCATGGCCACTGAGAG |
| | | TGTGGACGGAGAGCTGTCAGGCTGCAATGCCGCCATCCTCAAGCGGGAGACCATGAGG |
| | | CCATCCAGCCGTGTGGCCCTGATGGTGCTCTGTGAGACCCACCGCGCCCGCATGGTCAA |
| | | ACACCACTGCTGCCCGGGCTGCGGCTACTTCTGCACGGCGGGCACCTTCCTGGAGTGC |
| | | CACCCTGACTTCCGTGTGGCCCACCGCTTCCACAAGGCCTGTGTGTCTCAGCTGAATGG |
| | | GATGGTCTTCTGTCCCCACTGTGGGGAGGATGCTTCTGAAGCTCAAGAGGTGACCATCC |
| | | CCCGGGGTGACGGGGTGACCCCACCGGCCGGCACTGCAGCTCCTGCACCCCCACCCCT |
| | | GTCCCAGGATGTCCCCGGGAGAGCAGACACTTCTCAGCCCAGTGCCCGGATGCGAGGG |
| | | CATGGGGAACCCCGGCGCCCGCCCTGCGATCCCCTGGCTGACACCATTGACAGCTCAG |
| | | GGCCCTCCCTGACCCTGCCCAATGGGGGCTGCCTTTCAGCCGTGGGGCTGCCACTGGG |
| | | GCCAGGCCGGGAGGCCCTGGAAAAGGCCCTGGTCATCCAGGAGTCAGAGAGGCGGAA |
| | | GAAGCTCCGTTTCCACCCTCGGCAGTTGTACCTGTCCGTGAAGCAGGGCGAGCTGCAGA |
| | | AGGTGATCCTGATGCTGTTGGACAACCTGGACCCCAACTTCCAGAGCGACCAGCAGAGC |
| | | AAGCGCACGCCCCTGCATGCAGCCGCCCAGAAGGGCTCCGTGGAGATCTGCCATGTGC |
| | | TGCTGCAGGCTGGAGCCAACATAAATGCAGTGGACAAACAGCAGCGGACGCCACTGAT |
| | | GGAGGCCGTGGTGAACAACCACCTGGAGGTAGCCCGTTACATGGTGCAGCGTGGTGGC |
| | | TGTGTCTATAGCAAGGAGGAGGACGGTTCCACCTGCCTCCACCACGCAGCCAAAATCGG |
| | | GAACTTGGAGATGGTCAGCCTGCTGCTGAGCACAGGACAGGTGGACGTCAACGCCCAG |
| | | GACAGTGGGGGGTGGACGCCCATCATCTGGGCTGCAGAGCACAAGCACATCGAGGTGA |
| | | TCCGCATGCTACTGACGCGGGGCGCCGACGTCACCCTCACTGACAACGAGGAGAACAT |
| | | CTGCCTGCACTGGGCCTCCTTCACGGGCAGCGCCGCCATCGCCGAAGTCCTTCTGAATG |
| | | CGCGCTGTGACCTCCATGCTGTCAACTACCATGGGGACACCCCCCTGCACATCGCAGCT |
| | | CGGGAGAGCTACCATGACTGCGTGCTGTTATTCCTGTCACGTGGGGCCAACCCTGAGCT |
| | | GCGGAACAAAGAGGGGGACACAGCATGGGACCTGACTCCCGAGCGCTCCGACGTGTGG |
| | | TTTGCGCTTCAACTCAACCGCAAGCTCCGACTTGGGGTGGGAAATCGGGCCATCCGCAC |
| | | AGAGAAGATCATCTGCCGGGACGTGGCTCGGGGCTATGAGAACGTGCCCATTCCCTGT |
| | | GTCAACGGTGTGGATGGGGAGCCCTGCCCTGAGGATTACAAGTACATCTCAGAGAACTG |
| | | CGAGACGTCCACCATGAACATCGATCGCAACATCACCCACCTGCAGCACTGCACGTGTG |
| | | TGGACGACTGCTCTAGCTCCAACTGCCTGTGCGGCCAGCTCAGCATCCGGTGCTGGTAT |
| | | GACAAGGATGGGCGATTGCTCCAGGAATTTAACAAGATTGAGCCTCCGCTGATTTTCGA |
| | | GTGTAACCAGGCGTGCTCATGCTGGAGAAACTGCAAGAACCGGGTCGTACAGAGTGGC |
| | | ATCAAGGTGCGGCTACAGCTCTACCGAACAGCCAAGATGGGCTGGGGGGTCCGCGCCC |
| | | TGCAGACCATCCCACAGGGGACCTTCATCTGCGAGTATGTCGGGGAGCTGATCTCTGAT |
| | | GCTGAGGCTGATGTGAGAGAGGATGATTCTTACCTCTTCGACTTAGACAACAAGGATGG |
| | | AGAGGTGTACTGCATAGATGCCCGTTACTATGGCAACATCAGCCGCTTCATCAACCACCT |
| | | GTGTGACCCCAACATCATTCCCGTCCGGGTCTTCATGCTGCACCAAGACCTGCGATTTCC |
| | | ACGCATCGCCTTCTTCAGTTCCCGAGACATCCGGACTGGGGAGGAGCTAGGGTTTGACT |
| | | ATGGCGACCGCTTCTGGGACATCAAAAGCAAATATTTCACCTGCCAATGTGGCTCTGAG |
| | | AAGTGCAAGCACTCAGCCGAAGCCATTGCCCTGGAGCAGAGCCGTCTGGCCCGCCTGG |
| | | ACCCACACCCTGAGCTGCTGCCCGAGCTCGGCTCCCTGCCCCCTGTCAACACATGAGAA |
| | | CGGACCACACCCTCTCTCCCCAGCATGGATGGCCACAGCTCAGCCGCCTCCTCTGCCAC |
| | | CAGCTGCTCGCAGCCCATGCCTGGGGGTGCTGCCATCTTCTCTCCCCACCACCCTTTCA |
| | | CACATTCCTGACCAGAGATCCCAGCCAGGCCCTGGAGGTCTGACAGCCCCTCCCTCCCA |
| | | GAGCTGGTTCCTCCCTGGGAGGGCAACTTCAGGGCTGGCCACCCCCCGTGTTCCCCATC |
| | | CTCAGTTGAAGTTTGATGAATTGAAGTCGGGCCTCTATGCCAACTGGTTCCTTTTGTTCT |
| | | CAATAAATGTTGGGTTTGGTAATAAAAAAAAAAAAAAAAA |
| 8 | Human EHMT2 | MAAAAGAAAAAAAEGEAPAEMGALLLEKETRGATERVHGSLGDTPRSEETLPKATPDSLEP |
| | (G9a) | AGPSSPASVTVTVGDEGADTPVGATPLIGDESENLEGDGDLRGGRILLGHATKSFPSSPSK |
| | polypeptide, | GGSCPSRAKMSMTGAGKSPPSVQSLAMRLLSMPGAQGAAAAGSEPPPATTSPEGQPKVHR |
| | variant 3 (Acc. | ARKTMSKPGNGQPPVPEKRPPEIQHFRMSDDVHSLGKVTSDLAKRRKLNSGGGLSEELGS |
| | No. | ARRSGEVTLTKGDPGSLEEWETVVGDDFSLYYDSYSVDERVDSDSKSEVEALTEQLSEEEE |
| | NP_079532.5) | EEEEEEEEEEEEEEEEEEEEDEESGNQSDRSGSSGRRKAKKKWRKDSPWVKPSRKRRKRE |
| | | PPRAKEPRGVSNDTSSLETERGFEELPLCSCRMEAPKIDRISERAGHKCMATESVDGELSGC |
| | | NAAILKRETMRPSSRVALMVLCETHRARMVKHHCCPGCGYFCTAGTFLECHPDFRVAHRFH |
| | | KACVSQLNGMVFCPHCGEDASEAQEVTIPRGDGVTPPAGTAAPAPPPLSQDVPGRADTSQP |
| | | SARMRGHGEPRRPPCDPLADTIDSSGPSLTLPNGGCLSAVGLPLGPGREALEKALVIQESER |
| | | RKKLRFHPRQLYLSVKQGELQKVILMLLDNLDPNFQSDQQSKRTPLHAAAQKGSVEICHVLL |
| | | QAGANINAVDKQQRTPLMEAVVNNH EVARYMVQRGGCVYSKEEDGSTCLHHAAKIGNLE |
| | | MVSLLLSTGQVDVNAQDSGGWTPIIWAAEHKHIEVIRMLLTRGADVTLTDNEENICLHWAS |
| | | FTGSAAIAEVLLNARCDLHAVNYHGDTPLHIAARESYHDCVLLFLSRGANPELRNKEGDTAW |
| | | DLTPERSDVWFALQLNRKLRLGVGNRAIRTEKIICRDVARGYENVPIPCVNGVDGEPCPEDY |
| | | KYISENCETSTMNIDRNITHLQHCTCVDDCSSSNCLCGQLSIRCWYDKDGRLLQEFNKIEP |
| | | PLIFECNQACSCWRNCKNRVVQSGIKVRLQLYRTAKMGWGVRALQTIPQGTFICEYVGELI |
| | | SDAEADVREDDSYLFDLDNKDGEVYCIDARYYGNISRFINHLCDPNIIPVRVFMLHQDLRFP |
| | | RIAFFSSRDIRTGEELGFDYGDRFWDIKSKYFTCQCGSEKCKHSAEAIALEQSRLARLDPHP |
| | | ELLPELGSLPPVNT |
| 9 | Human EHMT2 | GGGGAGGCCCCCGCTGAGATGGGGGCGCTGCTGCTGGAGAAGGAAACCAGAGGAGCC |
| | (G9a) cDNA, | ACCGAGAGAGTTCATGGCTCTTTGGGGGACACCCCTCGTAGTGAAGAAACCCTGCCCAA |
| | transcript | GGCCACCCCCGACTCCCTGGAGCCTGCTGGCCCCTCATCTCCAGCCTCTGTCACTGTCA |
| | variant 4 (Acc. | CTGTTGGTGATGAGGGGGCTGACACCCCTGTAGGGGCTACACCACTCATTGGGGATGA |
| | No. | ATCTGAGAATCTTGAGGGAGATGGGGACCTCCGTGGGGGCCGGATCCTGCTGGGCCAT |
| | NM_001318833.1) | GCCACAAAGTCATTCCCCTCTTCCCCCAGCAAGGGGGGTTCCTGTCCTAGCCGGGCCAA |
| | | GATGTCAATGACAGGGGCGGGAAAATCACCTCCATCTGTCCAGAGTTTGGCTATGAGGC |
| | | TACTGAGTATGCCAGGAGCCCAGGGAGCTGCAGCAGCAGGGTCTGAACCCCCTCCAGC |
| | | CACCACGAGCCCAGAGGGACAGCCCAAGGTCCACCGAGCCCGCAAAACCATGTCCAAA |
| | | CCAGGAAATGGACAGCATACCAAGACCCCATCTCTAAAAGAAGTTTAAAAGAATGTTTCA |
| | | AAGGCCAGGCCCAGTGACTCACGCCTGTAATCCCGTACTTTCTGGGGAGGATCACTTGA |
| | | CACCAGGAGTTCAAGACCAGCCTGGGCAACATGGCAAGACCTCTTCTCTACCAAAAAAA |
| | | AAAATTAAGAAGACATTAGTTAGGCATTGTGACATGTGCCTGTAATCCCAGCTTCCCAGG |
| | | AAGCTGAGGCAGGAGAATGGCTTGAGTCCAGTTCGAGGCTGAAGTGAGCCATAGTCAT |
| | | GCCACTGCACTCCATCCTGGCCCCCGGTCCCTGAGAAGCGGCCCCCTGAAATACAGCAT |
| | | TTCCGCATGAGTGATGATGTCCACTCACTGGGAAAGGTGACCTCAGATCTGGCCAAAAG |
| | | GAGGAAGCTGAACTCAGGAGGTGGCCTGTCAGAGGAGTTAGGTTCTGCCCGGCGTTCA |
| | | GGAGAAGTGACCCTGACGAAAGGGGACCCCGGGTCCCTGGAGGAGTGGGAGACGGTG |
| | | GTGGGTGATGACTTCAGTCTCTACTATGATTCCTACTCTGTGGATGAGCGCGTGGACTCC |
| | | GACAGCAAGTCTGAAGTTGAAGCTCTAACTGAACAACTAAGTGAAGAGGAGGAGGAGG |
| | | AAGAGGAGGAAGAAGAAGAAGAGGAAGAGGAGGAGGAAGAGGAAGAAGAAGAGGAAG |
| | | ATGAGGAGTCAGGGAATCAGTCAGATAGGAGTGGTTCCAGTGGCCGGCGCAAGGCCAA |
| | | GAAGAAATGGCGAAAAGACAGCCCATGGGTGAAGCCGTCTCGGAAACGGCGCAAGCGG |
| | | GAGCCTCCGCGGGCCAAGGAGCCACGAGGAGTGAATGGTGTGGGCTCCTCAGGCCCCA |
| | | GTGAGTACATGGAGGTCCCTCTGGGGTCCCTGGAGCTGCCCAGCGAGGGGACCCTCTC |
| | | CCCCAACCACGCTGGGGTGTCCAATGACACATCTTCGCTGGAGACAGAGCGAGGGTTTG |
| | | AGGAGTTGCCCCTGTGCAGCTGCCGCATGGAGGCACCCAAGATTGACCGCATCAGCGA |
| | | GAGGGCGGGGCACAAGTGCATGGCCACTGAGAGTGTGGACGGAGAGCTGTCAGGCTG |
| | | CAATGCCGCCATCCTCAAGCGGGAGACCATGAGGCCATCCAGCCGTGTGGCCCTGATG |
| | | GTGCTCTGTGAGACCCACCGCGCCCGCATGGTCAAACACCACTGCTGCCCGGGCTGCG |
| | | GCTACTTCTGCACGGCGGGCACCTTCCTGGAGTGCCACCCTGACTTCCGTGTGGCCCAC |
| | | CGCTTCCACAAGGCCTGTGTGTCTCAGCTGAATGGGATGGTCTTCTGTCCCCACTGTGG |
| | | GGAGGATGCTTCTGAAGCTCAAGAGGTGACCATCCCCCGGGGTGACGGGGTGACCCCA |
| | | CCGGCCGGCACTGCAGCTCCTGCACCCCCACCCCTGTCCCAGGATGTCCCCGGGAGAG |
| | | CAGACACTTCTCAGCCCAGTGCCCGGATGCGAGGGCATGGGGAACCCCGGCGCCCGCC |
| | | CTGCGATCCCCTGGCTGACACCATTGACAGCTCAGGGCCCTCCCTGACCCTGCCCAATG |
| | | GGGGCTGCCTTTCAGCCGTGGGGCTGCCACTGGGGCCAGGCCGGGAGGCCCTGGAAA |
| | | AGGCCCTGGTCATCCAGGAGTCAGAGAGGCGGAAGAAGCTCCGTTTCCACCCTCGGCA |
| | | GTTGTACCTGTCCGTGAAGCAGGGCGAGCTGCAGAAGGTGATCCTGATGCTGTTGGACA |
| | | ACCTGGACCCCAACTTCCAGAGCGACCAGCAGAGCAAGCGCACGCCCCTGCATGCAGC |
| | | CGCCCAGAAGGGCTCCGTGGAGATCTGCCATGTGCTGCTGCAGGCTGGAGCCAACATA |
| | | AATGCAGTGGACAAACAGCAGCGGACGCCACTGATGGAGGCCGTGGTGAACAACCACC |
| | | TGGAGGTAGCCCGTTACATGGTGCAGCGTGGTGGCTGTGTCTATAGCAAGGAGGAGGA |
| | | CGGTTCCACCTGCCTCCACCACGCAGCCAAAATCGGGAACTTGGAGATGGTCAGCCTGC |
| | | TGCTGAGCACAGGACAGGTGGACGTCAACGCCCAGGACAGTGGGGGGTGGACGCCCAT |
| | | CATCTGGGCTGCAGAGCACAAGCACATCGAGGTGATCCGCATGCTACTGACGCGGGGC |
| | | GCCGACGTCACCCTCACTGACAACGTGAGTGAGCGTTTGGTTGAGGAGGAGAACATCTG |
| | | CCTGCACTGGGCCTCCTTCACGGGCAGCGCCGCCATCGCCGAAGTCCTTCTGAATGCGC |
| | | GCTGTGACCTCCATGCTGTCAACTACCATGGGGACACCCCCCTGCACATCGCAGCTCGG |
| | | GAGAGCTACCATGACTGCGTGCTGTTATTCCTGTCACGTGGGGCCAACCCTGAGCTGCG |
| | | GAACAAAGAGGGGGACACAGCATGGGACCTGACTCCCGAGCGCTCCGACGTGTGGTTT |
| | | GCGCTTCAACTCAACCGCAAGCTCCGACTTGGGGTGGGAAATCGGGCCATCCGCACAG |
| | | AGAAGATCATCTGCCGGGACGTGGCTCGGGGCTATGAGAACGTGCCCATTCCCTGTGTC |
| | | AACGGTGTGGATGGGGAGCCCTGCCCTGAGGATTACAAGTACATCTCAGAGAACTGCGA |
| | | GACGTCCACCATGAACATCGATCGCAACATCACCCACCTGCAGCACTGCACGTGTGTGG |
| | | ACGACTGCTCTAGCTCCAACTGCCTGTGCGGCCAGCTCAGCATCCGGTGCTGGTATGAC |
| | | AAGGATGGGCGATTGCTCCAGGAATTTAACAAGATTGAGCCTCCGCTGATTTTCGAGTG |
| | | TAACCAGGCGTGCTCATGCTGGAGAAACTGCAAGAACCGGGTCGTACAGAGTGGCATCA |
| | | AGGTGCGGCTACAGCTCTACCGAACAGCCAAGATGGGCTGGGGGGTCCGCGCCCTGCA |
| | | GACCATCCCACAGGGGACCTTCATCTGCGAGTATGTCGGGGAGCTGATCTCTGATGCTG |
| | | AGGCTGATGTGAGAGAGGATGATTCTTACCTCTTCGACTTAGACAACAAGGATGGAGAG |
| | | GTGTACTGCATAGATGCCCGTTACTATGGCAACATCAGCCGCTTCATCAACCACCTGTGT |
| | | GACCCCAACATCATTCCCGTCCGGGTCTTCATGCTGCACCAAGACCTGCGATTTCCACGC |
| | | ATCGCCTTCTTCAGTTCCCGAGACATCCGGACTGGGGAGGAGCTAGGGTTTGACTATGG |
| | | CGACCGCTTCTGGGACATCAAAAGCAAATATTTCACCTGCCAATGTGGCTCTGAGAAGT |
| | | GCAAGCACTCAGCCGAAGCCATTGCCCTGGAGCAGAGCCGTCTGGCCCGCCTGGACCC |
| | | ACACCCTGAGCTGCTGCCCGAGCTCGGCTCCCTGCCCCCTGTCAACACATGAGAACGGA |
| | | CCACACCCTCTCTCCCCAGCATGGATGGCCACAGCTCAGCCGCCTCCTCTGCCACCAGC |
| | | TGCTCGCAGCCCATGCCTGGGGGTGCTGCCATCTTCTCTCCCCACCACCCTTTCACACAT |
| | | TCCTGACCAGAGATCCCAGCCAGGCCCTGGAGGTCTGACAGCCCCTCCCTCCCAGAGCT |
| | | GGTTCCTCCCTGGGAGGGCAACTTCAGGGCTGGCCACCCCCCGTGTTCCCCATCCTCAG |
| | | TTGAAGTTTGATGAATTGAAGTCGGGCCTCTATGCCAACTGGTTCCTTTTGTTCTCAATAA |
| | | ATGTTGGGTTTGGTAATAAAAAAAAAAAAAAAAA |
| 10 | Human EHMT2 | MSDDVHSLGKVTSDLAKRRKLNSGGGLSEELGSARRSGEVTLTKGDPGSLEEWETVVGDD |
| | (G9a) | FSLYYDSYSVDERVDSDSKSEVEALTEQLSEEEEEEEEEEEEEEEEEEEEEEEEDEESGNQS |
| | polypeptide, | DRSGSSGRRKAKKKWRKDSPWVKPSRKRRKREPPRAKEPRGVNGVGSSGPSEYMEVPLG |
| | variant 4 (Acc. | SLELPSEGTLSPNHAGVSNDTSSLETERGFEELPLCSCRMEAPKIDRISERAGHKCMATESV |
| | No. | DGELSGCNAAILKRETMRPSSRVALMVLCETHRARMVKHHCCPGCGYFCTAGTFLECHPDF |
| | NP_001305762.1) | RVAHRFHKACVSQLNGMVFCPHCGEDASEAQEVTIPRGDGVTPPAGTAAPAPPPLSQDVPG |
| | | RADTSQPSARMRGHGEPRRPPCDPLADTIDSSGPSLTLPNGGCLSAVGLPLGPGREALEKA |
| | | LVIQESERRKKLRFHPRQLYLSVKQGELQKVILMLLDNLDPNFQSDQQSKRTPLHAAAQKG |
| | | SVEICHVLLQAGANINAVDKQQRTPLMEAVVNNHLEVARYMVQRGGCVYSKEEDGSTCLH |
| | | HAAKIGNLEMVSLLLSTGQVDVNAQDSGGWTPIIWAAEHKHIEVIRMLLTRGADVTLIDNV |
| | | SERLVEEENICLHWASFTGSAAIAEVLLNARCDLHAVNYHGDTPLHIAARESYHDCVLLFLS |
| | | RGANPELRNKEGDTAWDLTPERSDVWFALQLNRKLRLGVGNRAIRTEKIICRDVARGYENV |
| | | PIPCVNGVDGEPCPEDYKYISENCETSTMNIDRNITHLQHCTCVDDCSSSNCLCGQLSIRC |
| | | WYDKDGRLLQEFNKIEPPLIFECNQACSCWRNCKNRVVQSGIKVRLQLYRTAKMGWGVRA |
| | | LQTIPQGTFICEYVGELISDAEADVREDDSYLFDLDNKDGEVYCIDARYYGNISRFINHLCDP |
| | | NIIPVRVFMLHQD RFPRIAFFSSRDIRTGEELGFDYGDRFWDIKSKYFTCQCGSEKCKHSA |
| | | EAIALEQSRLARLDPHPELLPELGSLPPVNT |
| 11 | Human EHMT2 | AGAGATGCGGGGTCTACCGAGAGGGAGGGGGTTGATGCGGGCCCGGGGGAGGGGTCG |
| | (G9a) cDNA, | TGCGGCCCCTCCGGGCAGCCGAGGCCGCGGAAGGGGGGGGCCCCACAGAGGAAGAG |
| | transcript | GTAGGCCCCGGAGCCTACTCTCTCTTCCCAGGGCCCAGGCATCCTGGACCCCCCAACTC |
| | variant 5 (Acc. | TCTACTGGGCTGACCAGCCCTCCTGTCCCTTGTCTCCCCTCCCAGGGGGAGGCCCCCGC |
| | No. | TGAGATGGGGGCGCTGCTGCTGGAGAAGGAAACCAGAGGAGCCACCGAGAGAGTTCAT |
| | NM_001363689.1) | GGCTCTTTGGGGGACACCCCTCGTAGTGAAGAAACCCTGCCCAAGGCCACCCCCGACTC |
| | | CCTGGAGCCTGCTGGCCCCTCATCTCCAGCCTCTGTCACTGTCACTGTTGGTGATGAGG |
| | | GGGCTGACACCCCTGTAGGGGCTACACCACTCATTGGGGATGAATCTGAGAATCTTGAG |
| | | GGAGATGGGGACCTCCGTGGGGGCCGGATCCTGCTGGGCCATGCCACAAAGTCATTCC |
| | | CCTCTTCCCCCAGCAAGGGGGGTTCCTGTCCTAGCCGGGCCAAGATGTCAATGACAGGG |
| | | GCGGGAAAATCACCTCCATCTGTCCAGAGTTTGGCTATGAGGCTACTGAGTATGCCAGG |
| | | AGCCCAGGGAGCTGCAGCAGCAGGGTCTGAACCCCCTCCAGCCACCACGAGCCCAGAG |
| | | GGACAGCCCAAGGTCCACCGAGCCCGCAAAACCATGTCCAAACCAGGAAATGGACAGC |
| | | CCCCGGTCCCTGAGAAGCGGCCCCCTGAAATACAGCATTTCCGCATGAGTGATGATGTC |
| | | CACTCACTGGGAAAGGTGACCTCAGATCTGGCCAAAAGGAGGAAGCTGAACTCAGGAG |
| | | GTGGCCTGTCAGAGGAGTTAGGTTCTGCCCGGCGTTCAGGAGAAGTGACCCTGACGAA |
| | | AGGGGACCCCGGGTCCCTGGAGGAGTGGGAGACGGTGGTGGGTGATGACTTCAGTCTC |
| | | TACTATGATTCCTACTCTGTGGATGAGCGCGTGGACTCCGACAGCAAGTCTGAAGTTGA |
| | | AGCTCTAACTGAACAACTAAGTGAAGAGGAGGAGGAGGAAGAGGAGGAAGAAGAAGAA |
| | | GAGGAAGAGGAGGAGGAAGAGGAAGAAGAAGAGGAAGATGAGGAGTCAGGGAATCAG |
| | | TCAGATAGGAGTGGTTCCAGTGGCCGGCGCAAGGCCAAGAAGAAATGGCGAAAAGACA |
| | | GCCCATGGGTGAAGCCGTCTCGGAAACGGCGCAAGCGGGAGCCTCCGCGGGCCAAGG |
| | | AGCCACGAGGAGTGAATGGTGTGGGCTCCTCAGGCCCCAGTGAGTACATGGAGGTCCC |
| | | TCTGGGGTCCCTGGAGCTGCCCAGCGAGGGGACCCTCTCCCCCAACCACGCTGGGGTG |
| | | TCCAATGACACATCTTCGCTGGAGACAGAGCGAGGGTTTGAGGAGTTGCCCCTGTGCAG |
| | | CTGCCGCATGGAGGCACCCAAGATTGACCGCATCAGCGAGAGGGCGGGGCACAAGTGC |
| | | ATGGCCACTGAGAGTGTGGACGGAGAGCTGTCAGGCTGCAATGCCGCCATCCTCAAGC |
| | | GGGAGACCATGAGGCCATCCAGCCGTGTGGCCCTGATGGTGCTCTGTGAGACCCACCG |
| | | CGCCCGCATGGTCAAACACCACTGCTGCCCGGGCTGCGGCTACTTCTGCACGGCGGGC |
| | | ACCTTCCTGGAGTGCCACCCTGACTTCCGTGTGGCCCACCGCTTCCACAAGGCCTGTGT |
| | | GTCTCAGCTGAATGGGATGGTCTTCTGTCCCCACTGTGGGGAGGATGCTTCTGAAGCTC |
| | | AAGAGGTGACCATCCCCCGGGGTGACGGGGTGACCCCACCGGCCGGCACTGCAGCTCC |
| | | TGCACCCCCACCCCTGTCCCAGGATGTCCCCGGGAGAGCAGACACTTCTCAGCCCAGTG |
| | | CCCGGATGCGAGGGCATGGGGAACCCCGGCGCCCGCCCTGCGATCCCCTGGCTGACAC |
| | | CATTGACAGCTCAGGGCCCTCCCTGACCCTGCCCAATGGGGGCTGCCTTTCAGCCGTGG |
| | | GGCTGCCACTGGGGCCAGGCCGGGAGGCCCTGGAAAAGGCCCTGGTCATCCAGGAGTC |
| | | AGAGAGGCGGAAGAAGCTCCGTTTCCACCCTCGGCAGTTGTACCTGTCCGTGAAGCAG |
| | | GGCGAGCTGCAGAAGGTGATCCTGATGCTGTTGGACAACCTGGACCCCAACTTCCAGAG |
| | | CGACCAGCAGAGCAAGCGCACGCCCCTGCATGCAGCCGCCCAGAAGGGCTCCGTGGAG |
| | | ATCTGCCATGTGCTGCTGCAGGCTGGAGCCAACATAAATGCAGTGGACAAACAGCAGCG |
| | | GACGCCACTGATGGAGGCCGTGGTGAACAACCACCTGGAGGTAGCCCGTTACATGGTG |
| | | CAGCGTGGTGGCTGTGTCTATAGCAAGGAGGAGGACGGTTCCACCTGCCTCCACCACG |
| | | CAGCCAAAATCGGGAACTTGGAGATGGTCAGCCTGCTGCTGAGCACAGGACAGGTGGA |
| | | CGTCAACGCCCAGGACAGTGGGGGGTGGACGCCCATCATCTGGGCTGCAGAGCACAAG |
| | | CACATCGAGGTGATCCGCATGCTACTGACGCGGGGCGCCGACGTCACCCTCACTGACAA |
| | | CGAGGAGAACATCTGCCTGCACTGGGCCTCCTTCACGGGCAGCGCCGCCATCGCCGAA |
| | | GTCCTTCTGAATGCGCGCTGTGACCTCCATGCTGTCAACTACCATGGGGACACCCCCCT |
| | | GCACATCGCAGCTCGGGAGAGCTACCATGACTGCGTGCTGTTATTCCTGTCACGTGGGG |
| | | CCAACCCTGAGCTGCGGAACAAAGAGGGGGACACAGCATGGGACCTGACTCCCGAGCG |
| | | CTCCGACGTGTGGTTTGCGCTTCAACTCAACCGCAAGCTCCGACTTGGGGTGGGAAATC |
| | | GGGCCATCCGCACAGAGAAGATCATCTGCCGGGACGTGGCTCGGGGCTATGAGAACGT |
| | | GCCCATTCCCTGTGTCAACGGTGTGGATGGGGAGCCCTGCCCTGAGGATTACAAGTACA |
| | | TCTCAGAGAACTGCGAGACGTCCACCATGAACATCGATCGCAACATCACCCACCTGCAG |
| | | CACTGCACGTGTGTGGACGACTGCTCTAGCTCCAACTGCCTGTGCGGCCAGCTCAGCAT |
| | | CCGGTGCTGGTATGACAAGGATGGGCGATTGCTCCAGGAATTTAACAAGATTGAGCCTC |
| | | CGCTGATTTTCGAGTGTAACCAGGCGTGCTCATGCTGGAGAAACTGCAAGAACCGGGTC |
| | | GTACAGAGTGGCATCAAGGTGCGGCTACAGCTCTACCGAACAGCCAAGATGGGCTGGG |
| | | GGGTCCGCGCCCTGCAGACCATCCCACAGGGGACCTTCATCTGCGAGTATGTCGGGGA |
| | | GCTGATCTCTGATGCTGAGGCTGATGTGAGAGAGGATGATTCTTACCTCTTCGACTTAGA |
| | | CAACAAGGATGGAGAGGTGTACTGCATAGATGCCCGTTACTATGGCAACATCAGCCGCT |
| | | TCATCAACCACCTGTGTGACCCCAACATCATTCCCGTCCGGGTCTTCATGCTGCACCAAG |
| | | ACCTGCGATTTCCACGCATCGCCTTCTTCAGTTCCCGAGACATCCGGACTGGGGAGGAG |
| | | CTAGGGTTTGACTATGGCGACCGCTTCTGGGACATCAAAAGCAAATATTTCACCTGCCAA |
| | | TGTGGCTCTGAGAAGTGCAAGCACTCAGCCGAAGCCATTGCCCTGGAGCAGAGCCGTCT |
| | | GGCCCGCCTGGACCCACACCCTGAGCTGCTGCCCGAGCTCGGCTCCCTGCCCCCTGTCA |
| | | ACACATGAGAACGGACCACACCCTCTCTCCCCAGCATGGATGGCCACAGCTCAGCCGCC |
| | | TCCTCTGCCACCAGCTGCTCGCAGCCCATGCCTGGGGGTGCTGCCATCTTCTCTCCCCA |
| | | CCACCCTTTCACACATTCCTGACCAGAGATCCCAGCCAGGCCCTGGAGGTCTGACAGCC |
| | | CCTCCCTCCCAGAGCTGGTTCCTCCCTGGGAGGGCAACTTCAGGGCTGGCCACCCCCCG |
| | | TGTTCCCCATCCTCAGTTGAAGTTTGATGAATTGAAGTCGGGCCTCTATGCCAACTGGTT |
| | | CCTTTTGTTCTCAATAAATGTTGGGTTTGGTAATAAA |
| 12 | Human EHMT2 | MRGLPRGRGLMRARGRGRAAPPGSRGRGRGGPHRGRGRPRSLLSLPRAQASWTPQLSTG |
| | (G9a) | LTSPPVPCLPSQGEAPAEMGALLLEKETRGATERVHGSLGDTPRSEETLPKATPDSLEPAGPS |
| | polypeptide, | SPASVTVTVGDEGADTPVGATPLIGDESENLEGDGDLRGGRILLGHATKSFPSSPSKGGSC |
| | variant 5 (Acc. | PSRAKMSMTGAGKSPPSVQSLAMRLLSMPGAQGAAAAGSEPPPATTSPEGQPKVHRARKT |
| | No. | MSKPGNGQPPVPEKRPPEIQHFRMSDDVHSLGKVTSDLAKRRKLNSGGGLSEELGSARRS |
| | NP_001350618.1) | GEVTLIKGDPGSLEEWETVVGDDFSLYYDSYSVDERVDSDSKSEVEALTEQLSEEEEEEEE |
| | | EEEEEEEEEEEEEEEEDEESGNQSDRSGSSGRRKAKKKWRKDSPWVKPSRKRRKREPPRA |
| | | KEPRGVNGVGSSGPSEYMEVPLGSLELPSEGTLSPNHAGVSNDTSSLETERGFEELPLCSCR |
| | | MEAPKIDRISERAGHKCMATESVDGELSGCNAAILKRETMRPSSRVALMVLCETHRARMVK |
| | | HHCCPGCGYFCTAGTFLECHPDFRVAHRFHKACVSQLNGMVFCPHCGEDASEAQEVTIPR |
| | | GDGVTPPAGTAAPAPPPLSQDVPGRADTSQPSARMRGHGEPRRPPCDPLADTIDSSGPSLT |
| | | LPNGGCLSAVGLPLGPGREALEKALVIQESERRKKLRFHPRQLYLSVKQGELQKVILMLLDNL |
| | | DPNFQSDQQSKRTPLHAAAQKGSVEICHVLLQAGANINAVDKQQRTPLMEAVVNNHLEVA |
| | | RYMVQRGGCVYSKEEDGSTCLHHAAKIGNLEMVSLLLSTGQVDVNAQDSGGWTPIIWAAE |
| | | HKHIEVIRMLLTRGADVTLTDNEENICLHWASFTGSAAIAEVLLNARCDLHAVNYHGDTPLH |
| | | IAARESYHDCVLLFLSRGANPELRNKEGDTAWDLTPERSDVWFALQLNRKLRLGVGNRAIR |
| | | TEKIICRDVARGYENVPIPCVNGVDGEPCPEDYKYISENCETSTMNIDRNITHLQHCTCVDD |
| | | CSSSNCLCGQLSIRCWYDKDGRLLQEFNKIEPPLIFECNQACSCWRNCKNRVVQSGIKVRL |
| | | QLYRTAKMGWGVRALQTIPQGTFICEYVGELISDAEADVREDDSYLFDLDNKDGEVYCIDA |
| | | RYYGNISRFINHLCDPNIIPVRVFMLHQDLRFPRIAFFSSRDIRTGEELGFDYGDRFWDIKSK |
| | | YFTCQCGSEKCKHSAEAIALEQSRLARLDPHPELLPELGSLPPVNT |
| 13 | hMAP1LC3B F | AGGAGATACAAGGGAAGTGGCT |
| | primer (for | |
| | ChiP) | |
| 14 | hMAP1LC3B R | TTGAAGGTCTTCTCCGACGGCAT |
| | primer (for | |
| | ChiP) | |
| 15 | hMAP1LC3B F | ATCTTGGCTCACTGCAACCT |
| | 5 kb primer (for | |
| | ChiP) | |
| 16 | hMAP1LC3B R | GTTTGCGTCCTTTCCCTGTA |
| | 5 kb primer (for | |
| | ChiP) | |
| 17 | RT hMAP1LC3B | GATGTCCGACTTATTCGAGAGC |
| | F primer (for | |
| | qPCR) | |
| 18 | RT hMAP1LC3B | TTGAGCTGTAAGCGCCTTCTA |
| | R primer (for | |
| | qPCR) | |
| 19 | RT hBeclin1 F | GGCTGAGAGACTGGATCAGG |
| | primer (for | |
| | qPCR) | |
| 20 | RT hBeclin1 R | CTGCGTCTGGGCATAACG |
| | primer (for | |
| | qPCR) | |
| 21 | RT hBNIP3L F | CGTCTTCCATCCACAATGGAG |
| | primer (for | |
| | qPCR) | |
| 22 | RT hBNIP3L R | TTGTGGTGAAGGGCTGTCAC |
| | primer (for | |
| | qPCR) | |
| 23 | RT hHPRT F | TGCAGACTTTGCTTTCCTTGGTCAGG |
| | primer (for | |
| | qPCR) | |
| 24 | RT hHPRT R | CCAACACTTCGTGGGGTCCTTTTCA |
| | primer (for | |
| | qPCR) | |
| 25 | Human EHMT1 | GCGCGGGAGGGGCGGGGCCACGCTGCGGGCCCGGGCCATGGCCGCCGCCGATGCCG |
| | (GLP) cDNA | AGGCAGTTCCGGCGAGGGGGGAGCCTCAGCAGGATTGCTGTGTGAAAACCGAGCTGCT |
| | (Acc. No. | GGGAGAAGAGACACCTATGGCTGCCGATGAAGGCTCAGCAGAGAAACAGGCAGGAGAG |
| | NM_024757.4) | GCCCACATGGCTGCGGACGGTGAGACCAATGGGTCTTGTGAAAACAGCGATGCCAGCA |
| | | GTCATGCAAATGCTGCAAAGCACACTCAGGACAGCGCAAGGGTCAACCCCCAGGATGG |
| | | CACCAACACACTAACTCGGATAGCGGAAAATGGGGTTTCAGAAAGAGACTCAGAAGCGG |
| | | CGAAGCAAAACCACGTCACTGCCGACGACTTTGTGCAGACTTCTGTCATCGGCAGCAAC |
| | | GGATACATCTTAAATAAGCCGGCCCTACAGGCACAGCCCTTGAGGACTACCAGCACTCT |
| | | GGCCTCTTCGCTGCCTGGCCATGCTGCAAAAACCCTTCCTGGAGGGGCTGGCAAAGGCA |
| | | GGACTCCAAGCGCTTTTCCCCAGACGCCAGCCGCCCCACCAGCCACCCTTGGGGAGGG |
| | | GAGTGCTGACACAGAGGACAGGAAGCTCCCGGCCCCTGGCGCCGACGTCAAGGTCCAC |
| | | AGGGCACGCAAGACCATGCCGAAGTCCGTCGTGGGCCTGCATGCAGCCAGTAAAGATC |
| | | CCAGAGAAGTTCGAGAAGCTAGAGATCATAAGGAACCAAAAGAGGAGATCAACAAAAAC |
| | | ATTTCTGACTTTGGACGACAGCAGCTTTTACCCCCCTTCCCATCCCTTCATCAGTCGCTAC |
| | | CTCAGAACCAGTGCTACATGGCCACCACAAAATCACAGACAGCTTGCTTGCCTTTTGTTT |
| | | TAGCAGCTGCAGTATCTCGGAAGAAAAAACGAAGAATGGGAACCTATAGCCTGGTTCCT |
| | | AAGAAAAAGACCAAAGTATTAAAACAGAGGACGGTGATTGAGATGTTTAAGAGCATAAC |
| | | TCATTCCACTGTGGGTTCCAAGGGGGAGAAGGACCTGGGCGCCAGCAGCCTGCACGTG |
| | | AATGGGGAGAGCCTGGAGATGGACTCGGATGAGGACGACTCAGAGGAGCTCGAGGAG |
| | | GACGACGGCCATGGTGCAGAGCAGGCGGCCGCGTTCCCCACAGAGGACAGCAGGACTT |
| | | CCAAGGAGAGCATGTCGGAGGCTGATCGCGCCCAGAAGATGGACGGGGAGTCCGAGG |
| | | AGGAGCAGGAGTCCGTGGACACCGGGGAGGAGGAGGAAGGCGGTGACGAGTCTGACC |
| | | TGAGTTCGGAATCCAGCATTAAGAAGAAATTTCTCAAGAGGAAAGGAAAGACCGACAGT |
| | | CCCTGGATCAAGCCAGCCAGGAAAAGGAGGCGGAGAAGTAGAAAGAAGCCCAGCGGTG |
| | | CCCTCGGTTCTGAGTCGTATAAGTCATCTGCAGGAAGCGCTGAGCAGACGGCACCAGGA |
| | | GACAGCACAGGGTACATGGAAGTTTCTCTGGACTCCCTGGATCTCCGAGTCAAAGGAAT |
| | | TCTGTCTTCACAAGCAGAAGGGTTGGCCAACGGTCCAGATGTGCTGGAGACAGACGGCC |
| | | TCCAGGAAGTGCCTCTCTGCAGCTGCCGGATGGAAACACCGAAGAGTCGAGAGATCACC |
| | | ACACTGGCCAACAACCAGTGCATGGCTACAGAGAGCGTGGACCATGAATTGGGCCGGT |
| | | GCACAAACAGCGTGGTCAAGTATGAGCTGATGCGCCCCTCCAACAAGGCCCCGCTCCTC |
| | | GTGCTGTGTGAAGACCACCGGGGCCGCATGGTGAAGCACCAGTGCTGTCCTGGCTGTG |
| | | GCTACTTCTGCACAGCGGGTAATTTTATGGAGTGTCAGCCCGAGAGCAGCATCTCTCAC |
| | | CGTTTCCACAAAGACTGTGCCTCTCGAGTCAATAACGCCAGCTATTGTCCCCACTGTGGG |
| | | GAGGAGAGCTCCAAGGCCAAAGAGGTGACGATAGCTAAAGCAGACACCACCTCGACCG |
| | | TGACACCAGTCCCCGGGCAGGAGAAGGGCTCGGCCCTGGAGGGCAGGGCCGACACCA |
| | | CAACGGGCAGTGCTGCCGGGCCACCACTCTCGGAGGACGACAAGCTGCAGGGTGCAGC |
| | | CTCCCACGTGCCCGAGGGCTTTGATCCAACGGGACCTGCTGGGCTTGGGAGGCCAACT |
| | | CCCGGCCTTTCCCAGGGACCAGGGAAGGAAACCTTGGAGAGCGCTCTCATCGCCCTCG |
| | | ACTCGGAAAAACCCAAGAAGCTTCGCTTCCACCCAAAGCAGCTGTACTTCTCCGCCAGG |
| | | CAAGGGGAGCTTCAGAAGGTGCTCCTCATGCTGGTGGACGGAATTGACCCCAACTTCAA |
| | | AATGGAGCACCAGAATAAGCGCTCTCCACTGCACGCCGCGGCAGAGGCTGGACACGTG |
| | | GACATCTGCCACATGCTGGTTCAGGCGGGCGCTAATATTGACACCTGCTCAGAAGACCA |
| | | GAGGACCCCGTTGATGGAAGCAGCCGAAAACAACCATCTGGAAGCAGTGAAGTACCTCA |
| | | TCAAGGCTGGGGCCCTGGTGGATCCCAAGGACGCAGAGGGCTCTACGTGTTTGCACCT |
| | | GGCTGCCAAGAAAGGCCACTACGAAGTGGTCCAGTACCTGCTTTCAAATGGACAGATGG |
| | | ACGTCAACTGTCAGGATGACGGAGGCTGGACACCCATGATCTGGGCCACAGAGTACAA |
| | | GCACGTGGACCTCGTGAAGCTGCTGCTGTCCAAGGGCTCTGACATCAACATCCGAGACA |
| | | ACGAGGAGAACATTTGCCTGCACTGGGCGGCGTTCTCCGGCTGCGTGGACATAGCCGA |
| | | GATCCTGCTGGCTGCCAAGTGCGACCTCCACGCCGTGAACATCCACGGAGACTCGCCAC |
| | | TGCACATTGCCGCCCGGGAGAACCGCTACGACTGTGTCGTCCTCTTTCTTTCTCGGGATT |
| | | CAGATGTCACCTTAAAGAACAAGGAAGGAGAGACGCCCCTGCAGTGTGCGAGCCTCAAC |
| | | TCTCAGGTGTGGAGCGCTCTGCAGATGAGCAAGGCTCTGCAGGACTCGGCCCCCGACA |
| | | GGCCCAGCCCCGTGGAGAGGATAGTGAGCAGGGACATCGCTCGAGGCTACGAGCGCAT |
| | | CCCCATCCCCTGTGTCAACGCCGTGGACAGCGAGCCATGCCCCAGCAACTACAAGTACG |
| | | TCTCTCAGAACTGCGTGACGTCCCCCATGAACATCGACAGAAATATCACTCATCTGCAGT |
| | | ACTGCGTGTGCATCGACGACTGCTCCTCCAGCAACTGCATGTGCGGCCAGCTCAGCATG |
| | | CGCTGCTGGTACGACAAGGATGGCCGGCTCCTGCCAGAGTTCAACATGGCGGAGCCTC |
| | | CCTTGATCTTCGAATGCAACCACGCGTGCTCCTGCTGGAGGAACTGCCGAAATCGCGTC |
| | | GTACAGAATGGTCTCAGGGCAAGGCTGCAGCTCTACCGGACGCGGGACATGGGCTGGG |
| | | GCGTGCGGTCCCTGCAGGACATCCCACCAGGCACCTTTGTCTGCGAGTATGTTGGGGAG |
| | | CTGATTTCAGACTCAGAAGCCGACGTTCGAGAGGAAGATTCTTACCTCTTTGATCTCGAC |
| | | AATAAGGACGGGGAGGTTTACTGCATCGACGCGCGGTTCTACGGGAACGTCAGCCGGT |
| | | TCATCAACCACCACTGCGAGCCCAACCTGGTGCCCGTGCGCGTGTTCATGGCCCACCAG |
| | | GACCTGCGGTTCCCCCGGATCGCCTTCTTCAGCACCCGCCTGATCGAGGCCGGCGAGC |
| | | AGCTCGGGTTTGACTATGGAGAGCGCTTCTGGGACATCAAAGGCAAGCTCTTCAGCTGC |
| | | CGCTGCGGCTCCCCCAAGTGCCGGCACTCGAGCGCGGCCCTGGCCCAGCGTCAGGCCA |
| | | GCGCGGCCCAGGAGGCCCAGGAGGACGGCTTGCCCGACACCAGCTCCGCGGCTGCCG |
| | | CCGACCCCCTATGAGACGCCGCCGGCCAGCGGGGCGCTCGGGAGCCAGGGACCGCCG |
| | | CGTCGCCGATTAGAGGACGAGGAGGAGAGATTCCGCACGCAACCGAAAGGGTCCTTCG |
| | | GGGCTGCGCCGCCGGCTTCCTGGAGGGGTCGGAGGTGAGGCTGCAGCCCCTGCGGGC |
| | | GGGTGTGGATGCCTCCCAGCCACCTTCCCAGACCTGCGGCCTCACCGCGGGCCCAGTG |
| | | CCCAGGCTGGAGCGCACACTTTGGTCCGCGCGCCAGAGACGCTGGGAGTCCGCACTGG |
| | | CATCACCTTCTGAGTTTCTGATGCTGATTTGTCGTTGCGAAGTTTCTCGTTTCTTCCTCTG |
| | | ACCTCCGAGGTCCCCGCTGCACCACGGGGTTGCTCTGTTCTCCTGTCCGGCCCAGACTC |
| | | TTCTGTGTGGCGCCGCCGAAGCCACCGTTAGCGCGAGCTGCTCCGTTCGCCCTGCCCAC |
| | | GGCCTGCGTGGCTGGGGCCGAGTCCCAGGGGCCGCACGGAGGGCACAGTCTCCTGTC |
| | | AGGCTCGGAGAGGTCAGGAGACCGACCCCACCACTAACTTTGGAGAAAATGTGGGTTTG |
| | | CTTTTTAAAGGAATCCTATATCTAGTCCTATATATCAAACCTCTAACTGACGTTTCTTTTCG |
| | | AGGAAGTGGCTTGGTGGGTGCAGCCCCCGCCGGTTCCGTTGACGCTGGCACCTTCTGTT |
| | | GATTTTTTAAGCCACATGCTATGATGAATAAACTGATTTATTTTCTACCATTACTGAACATT |
| | | AGGACAAACACAAAATAAAAAACAAAACACAGACAACGGTGCTGATTCTGGTGTGGTTTC |
| | | TACTCACCACGTGAAATAAACTATCAACTGTATAAAGAGAACAAAGTGATTTTAGAATAAA |
| | | ATGCAGGAAAAACTTTTTTAAAGATGTTAGTCTTGTAGCGTGAATAAATTTGCCATCACCT |
| | | TTTGTGTGGTGGCCTGGCAGGTCATATACTTTTTTTTGGCATATACCTTTTTAAAGACTGT |
| | | AATTAGTGCAGTAACAGTGGGGTTTTTTTTGTGCAACTCTTCTAAAAACATTCATAATGCA |
| | | GTCATGTTTATTTTTTTCTGTTAAAATGTTTTTGACAGTTTTAAGAGCAGTCTTTTGGCTCT |
| | | GACCATTTCTTGTTCTGTTTCCAATGAAATCAATAAAAAAAAAGAAGTACTTTAAAAAAAA |
| | | AAAAAAAAAA |
| 26 | Human EHMT1 | MAAADAEAVPARGEPQQDCCVKTELLGEETPMAADEGSAEKQAGEAHMAADGETNGSCE |
| | (GLP) | NSDASSHANAAKHTQDSARVNPQDGTNTLTRIAENGVSERDSEAAKQNHVTADDFVQTS |
| | polypeptide, | VIGSNGYILNKPALQAQPLRTTSTLASSLPGHAAKTLPGGAGKGRTPSAFPQTPAAPPATLGE |
| | (Acc. No. | GSADTEDRKLPAPGADVKVHRARKTMPKSVVGLHAASKDPREVREARDHKEPKEEINKNIS |
| | NP_079033.4 | DFGRQQLLPPFPSLHQSLPQNQCYMATTKSQTACLPFVLAAAVSRKKKRRMGTYSLVPKKK |
| | | TKVLKQRTVIEMFKSITHSTVGSKGEKDLGASSLHVNGESLEMDSDEDDSEELEEDDGHG |
| | | AEQAAAFPTEDSRTSKESMSEADRAQKMDGESEEEQESVDTGEEEEGGDESDLSSESSIK |
| | | KKFLKRKGKTDSPWIKPARKRRRRSRKKPSGALGSESYKSSAGSAEQTAPGDSTGYMEVS |
| | | LDSLDLRVKGILSSQAEGLANGPDVLETDGLQEVPLCSCRMETPKSREITTLANNQCMATES |
| | | VDHELGRCTNSVVKYELMRPSNKAPLLVLCEDHRGRMVKHQCCPGCGYFCTAGNFMECQP |
| | | ESSISHRFHKDCASRVNNASYCPHCGEESSKAKEVTIAKADTTSTVTPVPGQEKGSALEGR |
| | | ADTTTGSAAGPPLSEDDKLQGAASHVPEGFDPTGPAGLGRPTPGLSQGPGKETLESALIALD |
| | | SEKPKKLRFHPKQLYFSARQGELQKVLLMLVDGIDPNFKMEHQNKRSPLHAAAEAGHVDIC |
| | | HMLVQAGANIDTCSEDQRTPLMEAAENNHLEAVKYLIKAGALVDPKDAEGSTCLHLAAKKG |
| | | HYEVVQYLLSNGQMDVNCQDDGGWIPMIWATEYKHVDLVKLLLSKGSDINIRDNEENICL |
| | | HWAAFSGCVDIAEILLAAKCDLHAVNIHGDSPLHIAARENRYDCVVLFLSRDSDVTLKNKEG |
| | | ETPLQCASLNSQVWSALQMSKALQDSAPDRPSPVERIVSRDIARGYERIPIPCVNAVDSEPC |
| | | PSNYKYVSQNCVTSPMNIDRNITHLQYCVCIDDCSSSNCMCGQLSMRCWYDKDGRLLPEF |
| | | NMAEPPLIFECNHACSCWRNCRNRVVQNGLRARLQLYRTRDMGWGVRSLQDIPPGTFVCE |
| | | YVGELISDSEADVREEDSYLFDLDNKDGEVYCIDARFYGNVSRFINHHCEPNLVPVRVFMAH |
| | | QDLRFPRIAFFSTRLIEAGEQLGFDYGERFWDIKGKLFSCRCGSPKCRHSSAALAQRQASAA |
| | | QEAQEDGLPDTSSAAAADPL |
| 27 | Human L-lactate | MATLKDQLIYNLLKEEQTPQNKITVVGVGAVGMACAISILMKDLADELALVDVIEDKLKGEM |
| | dehydrogenase | MDLQHGSLFLRTPKIVSGKDYNVTANSKLVIITAGARQQEGESRLNLVQRNVNIFKFIIPNVV |
| | A chain (Uniprot | KYSPNCKLLIVSNPVDILTYVAWKISGFPKNRVIGSGCNLDSARFRYLMGERLGVHPLSCHG |
| | Acc. No. | WVLGEHGDSSVPVWSGMNVAGVSLKTLHPDLGTDKDKEQWKEVHKQVVESAYEVIKLKG |
| | P00338) | YTSWAIGLSVADLAESIMKNLRRVHPVSTMIKGLYGIKDDVFLSVPCILGQNGISDLVKVILT |
| | | SEEEARLKKSADTLWGIQKELQF |
| 28 | Human L-lactate | MATLKEKLIAPVAEEEATVPNNKITVVGVGQVGMACAISILGKSLADELALVDVLEDKLKGE |
| | dehydrogenase | MMDLQHGSLFLQTPKIVADKDYSVTANSKIVVVTAGVRQQEGESRLNLVQRNVNVFKFIIP |
| | B chain (Uniprot | QIVKYSPDCIIIVVSNPVDILTYVTWKLSGLPKHRVIGSGCNLDSARFRYLMAEKLGIHPSSC |
| | Acc. No. | HGWILGEHGDSSVAVWSGVNVAGVSLQELNPEMGTDNDSENWKEVHKMVVESAYEVIKL |
| | P07195) | KGYTNWAIGLSVADLIESMLKNLSRIHPVSTMVKGMYGIENEVFLSLPCILNARGLTSVINQ |
| | | KLKDDEVAQLKKSADTLWDIQKDLKDL |

1. A method for determining an indicator used in assessing a likelihood of a subject with cancer responding to cancer therapy, the method comprising, consisting or consisting essentially of:
(1) determining a biomarker value for at least one cancer therapy biomarker in a sample from the subject, wherein the, or one of the, cancer therapy biomarker(s) is an expression product of MAP1LC3B; and
(2) determining the indicator using the biomarker value(s), wherein the indicator is at least partially indicative of the likelihood of responsiveness to cancer therapy;
wherein the cancer therapy comprises therapy with an immune checkpoint inhibitor.

2.-43. (canceled)

44. A composition for determining an indicator used in assessing a likelihood of a subject with cancer responding to cancer therapy, the composition or solid support comprising, consisting, or consisting essentially of a MAP1LC3B transcript or cDNA thereof and at least one oligonucleotide primer or probe that hybridizes to the MAP1LC3B transcript or cDNA thereof, and an EHMT2 or EHMT1 transcript or cDNA thereof and at least one oligonucleotide primer or probe that hybridizes to the EHMT2 or EHMT1 transcript or cDNA thereof, wherein the cancer therapy comprises therapy with an immune checkpoint inhibitor.

45. A solid support for determining an indicator used in assessing a likelihood of a subject with cancer responding to cancer therapy, the solid support comprising, consisting, or consisting essentially of at least one first oligonucleotide primer or probe immobilized to the solid support, wherein the at least one first oligonucleotide primer or probe hybridizes to a MAP1LC3B transcript or cDNA; and at least one second oligonucleotide primer or probe immobilized to the solid support, wherein the at least one second oligonucleotide primer or probe hybridizes to a EHMT2 or EHMT1 transcript or cDNA thereof, wherein the cancer therapy comprises therapy with an immune checkpoint inhibitor.

46.-52. (canceled)


SEQUENCE LISTING (28 sequences)

SEQ ID NO: 1 - DNA, 2304 bp, Homo sapiens
acgctgcgtgccgctgctgggttccgccacgcccgtcatggcggcggccccggccggctctggccccgcccctcggtgacgcgtcgcgagtcacctgaccaggctgcgggctgaggagatacaagggaagtggctatcgccagagtcggattcgccgccgcagcagccgccgcccccgggagccgccgggaccctcgcgtcgtcgccgccgccgccgcccagatccctgcaccatgccgtcggagaagaccttcaagcagcgccgcaccttcgaacaaagagtagaagatgtccgacttattcgagagcagcatccaaccaaaatcccggtgataatagaacgatacaagggtgagaagcagcttcctgttctggataaaacaaagttccttgtacctgaccatgtcaacatgagtgagctcatcaagataattagaaggcgcttacagctcaatgctaatcaggccttcttcctgttggtgaacggacacagcatggtcagcgtctccacaccaatctcagaggtgtatgagagtgagaaagatgaagatggattcctgtacatggtctatgcctcccaggagacgttcgggatgaaattgtcagtgtaaaaccagaaaaaatgcagctcttctagaattgtttaaacccttaccaaggaaaaaaaagggatgttaccaactgagatcgatcagttcatccaatcacagatcatgaaacagtagtgttcccacctaggagtgttaggaagttgtgtttgtgtttcaagcagaaaaactgagctccaagtgagcacattcagctttggaaactatattatttaatgtaggctagcttgttttcaaattttaaaagtttaaaaataaaatactttgcattctaagttgccaataaaatagaccttcaagttattttaatgctcttttctcactaataggaacttgtaattccagcagtaatttaaaggctttcagagagaccctgagtcttctcttcaggttcacagaacccgccgcctttttgggtagaagttttctactcagctagagagatctccctaagaggatctttaggcctgagttgtgaagcgcaacccccgcaaaacgcatttgccatcacagttggcacaaacgcagggtaaacgggctgtgtgagaaaacggccctgactgtaaactgctgaaggtccctgactcctaagagaaccacacccaaagtcctcactcttgcaggggtagacatttctggtttggtttgttctctagatagttacacacataaagacaccactcaaaaggaaacttgaataatttataattttgatcgagtttcttaaaagaccctggagaaagagtggcatttcttctgtttcaggttttgtctgagttcaaactagtgcctgtgttgttacggaaagcagcagtgtaccagtgtcactctggagtacagcgggagaaacacaaaatagtataactgaaaacattaacattcagacacactcccttctgccttccggcttaaagctgtggatgatccacgtttttgtttttttaatgttaaatgtgtaactcagtattactgaaaaggtacccacattttgaatagtagttatcactcttaggtcagacagccatcagaattctcccacaccaagtgcatgtcagttgtggagaaaacatagcaaaaagagccgtacgctctttacagatactaatgtcaagagttaaacctcctcaggttcaacctgtgataaaagactagtgcttcccagtacttgcatggggttcactatttatagttttcttgggagtatcacaggaaaatcacaattacaccactttagaccctatgtgtagcaggtcacaacttacccttgtgtgtttagatgtgtatgaaatacctgtatacgttagtgaaagctgtttactgtaacggggaaaaccagattctttgcatctgggccctctactgattgttaaaggagttcctgtcacctgctccccccacccccgcatgcgtctgtccacttggctaacttttaatatgtgtatttttacattatgtatattcttaactggactgtctcgtttagactgtatacatcatatctgacattattgtaactaccgtgtgatcagtaagattcctgtaagaaatactgctttttaagaaaaaaaataacatgctgaggggtgacctatatcccatgtgagtggtcactttatttataggatctttaaaacatttttaatgaactaagttgaataaaggcacaattaaaaactgtcaaaaaaaaaaaaaaaaa

SEQ ID NO: 2 - protein, 125 aa, Homo sapiens
MPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 3 - DNA, 4045 bp, Homo sapiens
agagatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 4 - protein, 1233 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 5 - DNA, 4002 bp, Homo sapiens
gcgccggctcggcccccagcgcaagcggcgatggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 6 - protein, 1210 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 7 - DNA, 3900 bp, Homo sapiens
gcgccggctcggcccccagcgcaagcggcgatggcggcggcggcgggagctgcagcggcggcggccgccgagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 8 - protein, 1176 aa, Homo sapiens
MAAAAGAAAAAAAEGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 9 - DNA, 4252 bp, Homo sapiens
ggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcataccaagaccccatctctaaaagaagtttaaaagaatgtttcaaaggccaggcccagtgactcacgcctgtaatcccgtactttctggggaggatcacttgacaccaggagttcaagaccagcctgggcaacatggcaagacctcttctctaccaaaaaaaaaaattaagaagacattagttaggcattgtgacatgtgcctgtaatcccagcttcccaggaagctgaggcaggagaatggcttgagtccagttcgaggctgaagtgagccatagtcatgccactgcactccatcctggcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgtgagtgagcgtttggttgaggaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaaaaaaaaaaaaaaaa

SEQ ID NO: 10 - protein, 1008 aa, Homo sapiens
MSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNVSERLVEEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 11 - DNA, 4133 bp, Homo sapiens
agagatgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggagtgaatggtgtgggctcctcaggccccagtgagtacatggaggtccctctggggtccctggagctgcccagcgaggggaccctctcccccaaccacgctggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatgagaacggaccacaccctctctccccagcatggatggccacagctcagccgcctcctctgccaccagctgctcgcagcccatgcctgggggtgctgccatcttctctccccaccaccctttcacacattcctgaccagagatcccagccaggccctggaggtctgacagcccctccctcccagagctggttcctccctgggagggcaacttcagggctggccaccccccgtgttccccatcctcagttgaagtttgatgaattgaagtcgggcctctatgccaactggttccttttgttctcaataaatgttgggtttggtaataaa

SEQ ID NO: 12 - protein, 1267 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVNGVGSSGPSEYMEVPLGSLELPSEGTLSPNHAGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 13 - DNA, 22 bp, Artificial Sequence
  synthetic
aggagatacaagggaagtggct

SEQ ID NO: 14 - DNA, 23 bp, Artificial Sequence
  synthetic
ttgaaggtcttctccgacggcat

SEQ ID NO: 15 - DNA, 20 bp, Artificial Sequence
  synthetic
atcttggctcactgcaacct

SEQ ID NO: 16 - DNA, 20 bp, Artificial Sequence
  synthetic
gtttgcgtcctttccctgta

SEQ ID NO: 17 - DNA, 22 bp, Artificial Sequence
  synthetic
gatgtccgacttattcgagagc

SEQ ID NO: 18 - DNA, 21 bp, Artificial Sequence
  synthetic
ttgagctgtaagcgccttcta

SEQ ID NO: 19 - DNA, 20 bp, Artificial Sequence
  synthetic
ggctgagagactggatcagg

SEQ ID NO: 20 - DNA, 18 bp, Artificial Sequence
  synthetic
ctgcgtctgggcataacg

SEQ ID NO: 21 - DNA, 21 bp, Artificial Sequence
  synthetic
cgtcttccatccacaatggag

SEQ ID NO: 22 - DNA, 20 bp, Artificial Sequence
  synthetic
ttgtggtgaagggctgtcac

SEQ ID NO: 23 - DNA, 26 bp, Artificial Sequence
  synthetic
tgcagactttgctttccttggtcagg

SEQ ID NO: 24 - DNA, 25 bp, Artificial Sequence
  synthetic
ccaacacttcgtggggtccttttca

SEQ ID NO: 25 - DNA, 5123 bp, Homo sapiens
gcgcgggaggggcggggccacgctgcgggcccgggccatggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagcttgcttgccttttgttttagcagctgcagtatctcggaagaaaaaacgaagaatgggaacctatagcctggttcctaagaaaaagaccaaagtattaaaacagaggacggtgattgagatgtttaagagcataactcattccactgtgggttccaagggggagaaggacctgggcgccagcagcctgcacgtgaatggggagagcctggagatggactcggatgaggacgactcagaggagctcgaggaggacgacggccatggtgcagagcaggcggccgcgttccccacagaggacagcaggacttccaaggagagcatgtcggaggctgatcgcgcccagaagatggacggggagtccgaggaggagcaggagtccgtggacaccggggaggaggaggaaggcggtgacgagtctgacctgagttcggaatccagcattaagaagaaatttctcaagaggaaaggaaagaccgacagtccctggatcaagccagccaggaaaaggaggcggagaagtagaaagaagcccagcggtgccctcggttctgagtcgtataagtcatctgcaggaagcgctgagcagacggcaccaggagacagcacagggtacatggaagtttctctggactccctggatctccgagtcaaaggaattctgtcttcacaagcagaagggttggccaacggtccagatgtgctggagacagacggcctccaggaagtgcctctctgcagctgccggatggaaacaccgaagagtcgagagatcaccacactggccaacaaccagtgcatggctacagagagcgtggaccatgaattgggccggtgcacaaacagcgtggtcaagtatgagctgatgcgcccctccaacaaggccccgctcctcgtgctgtgtgaagaccaccggggccgcatggtgaagcaccagtgctgtcctggctgtggctacttctgcacagcgggtaattttatggagtgtcagcccgagagcagcatctctcaccgtttccacaaagactgtgcctctcgagtcaataacgccagctattgtccccactgtggggaggagagctccaaggccaaagaggtgacgatagctaaagcagacaccacctcgaccgtgacaccagtccccgggcaggagaagggctcggccctggagggcagggccgacaccacaacgggcagtgctgccgggccaccactctcggaggacgacaagctgcagggtgcagcctcccacgtgcccgagggctttgatccaacgggacctgctgggcttgggaggccaactcccggcctttcccagggaccagggaaggaaaccttggagagcgctctcatcgccctcgactcggaaaaacccaagaagcttcgcttccacccaaagcagctgtacttctccgccaggcaaggggagcttcagaaggtgctcctcatgctggtggacggaattgaccccaacttcaaaatggagcaccagaataagcgctctccactgcacgccgcggcagaggctggacacgtggacatctgccacatgctggttcaggcgggcgctaatattgacacctgctcagaagaccagaggaccccgttgatggaagcagccgaaaacaaccatctggaagcagtgaagtacctcatcaaggctggggccctggtggatcccaaggacgcagagggctctacgtgtttgcacctggctgccaagaaaggccactacgaagtggtccagtacctgctttcaaatggacagatggacgtcaactgtcaggatgacggaggctggacacccatgatctgggccacagagtacaagcacgtggacctcgtgaagctgctgctgtccaagggctctgacatcaacatccgagacaacgaggagaacatttgcctgcactgggcggcgttctccggctgcgtggacatagccgagatcctgctggctgccaagtgcgacctccacgccgtgaacatccacggagactcgccactgcacattgccgcccgggagaaccgctacgactgtgtcgtcctctttctttctcgggattcagatgtcaccttaaagaacaaggaaggagagacgcccctgcagtgtgcgagcctcaactctcaggtgtggagcgctctgcagatgagcaaggctctgcaggactcggcccccgacaggcccagccccgtggagaggatagtgagcagggacatcgctcgaggctacgagcgcatccccatcccctgtgtcaacgccgtggacagcgagccatgccccagcaactacaagtacgtctctcagaactgcgtgacgtcccccatgaacatcgacagaaatatcactcatctgcagtactgcgtgtgcatcgacgactgctcctccagcaactgcatgtgcggccagctcagcatgcgctgctggtacgacaaggatggccggctcctgccagagttcaacatggcggagcctcccttgatcttcgaatgcaaccacgcgtgctcctgctggaggaactgccgaaatcgcgtcgtacagaatggtctcagggcaaggctgcagctctaccggacgcgggacatgggctggggcgtgcggtccctgcaggacatcccaccaggcacctttgtctgcgagtatgttggggagctgatttcagactcagaagccgacgttcgagaggaagattcttacctctttgatctcgacaataaggacggggaggtttactgcatcgacgcgcggttctacgggaacgtcagccggttcatcaaccaccactgcgagcccaacctggtgcccgtgcgcgtgttcatggcccaccaggacctgcggttcccccggatcgccttcttcagcacccgcctgatcgaggccggcgagcagctcgggtttgactatggagagcgcttctgggacatcaaaggcaagctcttcagctgccgctgcggctcccccaagtgccggcactcgagcgcggccctggcccagcgtcaggccagcgcggcccaggaggcccaggaggacggcttgcccgacaccagctccgcggctgccgccgaccccctatgagacgccgccggccagcggggcgctcgggagccagggaccgccgcgtcgccgattagaggacgaggaggagagattccgcacgcaaccgaaagggtccttcggggctgcgccgccggcttcctggaggggtcggaggtgaggctgcagcccctgcgggcgggtgtggatgcctcccagccaccttcccagacctgcggcctcaccgcgggcccagtgcccaggctggagcgcacactttggtccgcgcgccagagacgctgggagtccgcactggcatcaccttctgagtttctgatgctgatttgtcgttgcgaagtttctcgtttcttcctctgacctccgaggtccccgctgcaccacggggttgctctgttctcctgtccggcccagactcttctgtgtggcgccgccgaagccaccgttagcgcgagctgctccgttcgccctgcccacggcctgcgtggctggggccgagtcccaggggccgcacggagggcacagtctcctgtcaggctcggagaggtcaggagaccgaccccaccactaactttggagaaaatgtgggtttgctttttaaaggaatcctatatctagtcctatatatcaaacctctaactgacgtttcttttcgaggaagtggcttggtgggtgcagcccccgccggttccgttgacgctggcaccttctgttgattttttaagccacatgctatgatgaataaactgatttattttctaccattactgaacattaggacaaacacaaaataaaaaacaaaacacagacaacggtgctgattctggtgtggtttctactcaccacgtgaaataaactatcaactgtataaagagaacaaagtgattttagaataaaatgcaggaaaaacttttttaaagatgttagtcttgtagcgtgaataaatttgccatcaccttttgtgtggtggcctggcaggtcatatacttttttttggcatatacctttttaaagactgtaattagtgcagtaacagtggggttttttttgtgcaactcttctaaaaacattcataatgcagtcatgtttatttttttctgttaaaatgtttttgacagttttaagagcagtcttttggctctgaccatttcttgttctgtttccaatgaaatcaataaaaaaaaagaagtactttaaaaaaaaaaaaaaaaaa

SEQ ID NO: 26 - protein, 1298 aa, Homo sapiens
MAAADAEAVPARGEPQQDCCVKTELLGEETPMAADEGSAEKQAGEAHMAADGETNGSCENSDASSHANAAKHTQDSARVNPQDGTNTLTRIAENGVSERDSEAAKQNHVTADDFVQTSVIGSNGYILNKPALQAQPLRTTSTLASSLPGHAAKTLPGGAGKGRTPSAFPQTPAAPPATLGEGSADTEDRKLPAPGADVKVHRARKTMPKSVVGLHAASKDPREVREARDHKEPKEEINKNISDFGRQQLLPPFPSLHQSLPQNQCYMATTKSQTACLPFVLAAAVSRKKKRRMGTYSLVPKKKTKVLKQRTVIEMFKSITHSTVGSKGEKDLGASSLHVNGESLEMDSDEDDSEELEEDDGHGAEQAAAFPTEDSRTSKESMSEADRAQKMDGESEEEQESVDTGEEEEGGDESDLSSESSIKKKFLKRKGKTDSPWIKPARKRRRRSRKKPSGALGSESYKSSAGSAEQTAPGDSTGYMEVSLDSLDLRVKGILSSQAEGLANGPDVLETDGLQEVPLCSCRMETPKSREITTLANNQCMATESVDHELGRCTNSVVKYELMRPSNKAPLLVLCEDHRGRMVKHQCCPGCGYFCTAGNFMECQPESSISHRFHKDCASRVNNASYCPHCGEESSKAKEVTIAKADTTSTVTPVPGQEKGSALEGRADTTTGSAAGPPLSEDDKLQGAASHVPEGFDPTGPAGLGRPTPGLSQGPGKETLESALIALDSEKPKKLRFHPKQLYFSARQGELQKVLLMLVDGIDPNFKMEHQNKRSPLHAAAEAGHVDICHMLVQAGANIDTCSEDQRTPLMEAAENNHLEAVKYLIKAGALVDPKDAEGSTCLHLAAKKGHYEVVQYLLSNGQMDVNCQDDGGWTPMIWATEYKHVDLVKLLLSKGSDINIRDNEENICLHWAAFSGCVDIAEILLAAKCDLHAVNIHGDSPLHIAARENRYDCVVLFLSRDSDVTLKNKEGETPLQCASLNSQVWSALQMSKALQDSAPDRPSPVERIVSRDIARGYERIPIPCVNAVDSEPCPSNYKYVSQNCVTSPMNIDRNITHLQYCVCIDDCSSSNCMCGQLSMRCWYDKDGRLLPEFNMAEPPLIFECNHACSCWRNCRNRVVQNGLRARLQLYRTRDMGWGVRSLQDIPPGTFVCEYVGELISDSEADVREEDSYLFDLDNKDGEVYCIDARFYGNVSRFINHHCEPNLVPVRVFMAHQDLRFPRIAFFSTRLIEAGEQLGFDYGERFWDIKGKLFSCRCGSPKCRHSSAALAQRQASAAQEAQEDGLPDTSSAAAADPL

SEQ ID NO: 27 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 28 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL